SEQUENCE LISTING (59 sequences)

SEQ ID NO: 1 - DNA, 1527 bp, Zea mays
  source 1..1527: mol_type = genomic DNA
atgggttggctgaccaagttttttagaggctcaacccacaatatctcggaagagcagtatcacagcaggcctgcagaagacacagcatggaatgaaccctctagctctcctgttgtgactgacatcctttcagagtttaacaatgaggatattgaccgtgctatagcgctctctctatcggaagaggagcaaagaaaggaaaaggcaatagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcagagcatgcgcaggatgtcacagagagattggtcacgggcgttttctcagttgcatgggagctgtttggcatccggaatgttttcgttgccatgcatgtagtcaacctatatatgactatgagttctccatgtccgggaatcatccataccataaaacttgctacaaggagcagtttcacccgaaatgtgatgtctgcaagcagttcattcctacaaatatgaatggccttattgaatatagggcacatcctttctgggtacaaaaatactgtccttcacatgaaatggatggtactccgaggtgctgtagttgtgaaagaatggagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggctggtcaccaccatcttcctgaaacaagaggactatgcttatcagaagaacagactgttagcacgatactgagaccaagaatggctggaaacaaaattatgggaatgataacagagccatataggctgacacggcgatgtgaagtgactgcaattcttattctgtatggtctcccaagattgttgacaggttcaattttagctcatgagatgatgcatgcatggttgcggcttaaaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 2 - protein, 508 aa, Zea mays
MGWLTKFFRGSTHNISEEQYHSRPAEDTAWNEPSSSPVVTDILSEFNNEDIDRAIALSLSEEEQRKEKAIDKDMHLEEDEQLARAIQESLNVESPPRRNGSANGGTMYHPPRETGNAYQPPRENGSANGGNAYHPLPFMFSSGFRACAGCHREIGHGRFLSCMGAVWHPECFRCHACSQPIYDYEFSMSGNHPYHKTCYKEQFHPKCDVCKQFIPTNMNGLIEYRAHPFWVQKYCPSHEMDGTPRCCSCERMEPRESKYVLLDDGRKLCLECLDSAVMDTNDCQPLYLEIQEFYEGLNMKVEQQVPLLLVERQALNEAMEGEKAGHHHLPETRGLCLSEEQTVSTILRPRMAGNKIMGMITEPYRLTRRCEVTAILILYGLPRLLTGSILAHEMMHAWLRLKGYRTLSPDVEEGICQVLAHMWIESEIMAGSGSSAASSSSGSSSSTSSKKGGRSQFEHRLGDFFKHQIETDTSMAYGDGFRTGNRAVLHYGLKRTLEHIRLTGTFPF

SEQ ID NO: 3 - DNA, 6811 bp, Zea mays
  source 1..6811: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggctgtatgtttactatgctcaaaacccaaaatttctttttctaccccactgtactgttagctgtagctattttgtttcttttggataccagggtcaccaccatcttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 4 - DNA, 20 bp, unidentified
  source 1..20: mol_type = genomic DNA; note = Lachnospiraceae bacterium ND2006
aatttctactaagtgtagat

SEQ ID NO: 5 - DNA, 877 bp, Zea mays
  source 1..877: mol_type = genomic DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttcccc

SEQ ID NO: 6 - DNA, 3681 bp, unidentified
  source 1..3681: mol_type = genomic DNA; note = Lachnospiraceae bacterium ND2006
tcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 7 - DNA, 30 bp, Solanum lycopersicum
  source 1..30: mol_type = genomic DNA
ggatctaagaagagaagaattaaacaagat

SEQ ID NO: 8 - DNA, 23 bp, Zea mays
  source 1..23: mol_type = genomic DNA
gataccagggtcaccaccatctt

SEQ ID NO: 9 - DNA, 23 bp, Zea mays
  source 1..23: mol_type = genomic DNA
agcatagtaaacatacagccttc

SEQ ID NO: 10 - DNA, 23 bp, Zea mays
  source 1..23: mol_type = genomic DNA
gggtgaaactgctccttgtagca

SEQ ID NO: 11 - DNA, 6696 bp, Zea mays
  source 1..6696: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaagctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 12 - DNA, 6797 bp, Zea mays
  source 1..6797: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggatatgtttactatgctcaaaacccaaaatttctttttctaccccactgtactgttagctgtagctattttgtttcttttggataccagggtcaccacttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 13 - DNA, 6798 bp, Zea mays
  source 1..6798: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagtatgtttactatgctcaaaacccaaaatttctttttctaccccactgtactgttagctgtagctattttgtttcttttggataccagggtcaccatcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 14 - DNA, 6793 bp, Zea mays
  source 1..6793: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaaccaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagtttactatgctcaaaacccaaaatttctttttctaccccactgtactgttagctgtagctattttgtttcttttggataccagggtcaccaccatcttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 15 - DNA, 6795 bp, Zea mays
  source 1..6795: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggatatgtttactatgctcaaaacccaaaatttctttttctaccccactgtactgttagctgtagctattttgtttcttttggataccagggtcatcttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 16 - DNA, 6698 bp, Zea mays
  source 1..6698: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagtgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 17 - DNA, 6799 bp, Zea mays
  source 1..6799: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagctgtatgtttactatgctcaaaacccaaaatttctttttctaccccactgtactgttagctgtagctattttgtttcttttggataccagggtcaccttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 18 - DNA, 6779 bp, Zea mays
  source 1..6779: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagaccaaaatttctttttctaccccactgtactgttagctgtagctattttgtttcttttggataccagggtcaccatcttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 19 - DNA, 6701 bp, Zea mays
  source 1..6701: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggaggcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 20 - DNA, 6798 bp, Zea mays
  source 1..6798: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagatttactatgctcaaaacccaaaatttctttttctaccccactgtactgttagctgtagctattttgtttcttttggataccagggtcaccatcttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 21 - DNA, 6792 bp, Zea mays
  source 1..6792: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagatgtttactatgctcaaaacccaaaatttctttttctaccccactgtactgttagctgtagctattttgtttcttttggataccagggtcatcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 22 - DNA, 6703 bp, Zea mays
  source 1..6703: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagtcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 23 - DNA, 6788 bp, Zea mays
  source 1..6788: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggatgtttactatgctcaaaacccaaaatttctttttctaccccactgtactgttagctgtagctattttgtttcttttggataccagggtcactgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 24 - DNA, 6786 bp, Zea mays
  source 1..6786: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatgggtttactatgctcaaaacccaaaatttctttttctaccccactgtactgttagctgtagctattttgtttcttttggataccagggtcacctcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 25 - DNA, 6801 bp, Zea mays
  source 1..6801: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggctgtactatgctcaaaacccaaaatttctttttctaccccactgtactgttagctgtagctattttgtttcttttggataccagggtcaccacttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 26 - DNA, 6163 bp, Zea mays
  source 1..6163: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttactatgctcaaaacccaaaatttctttttctaccccactgtactgttagctgtagctattttgtttcttttggtgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 27 - DNA, 6803 bp, Zea mays
  source 1..6803: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagatgtttactatgctcaaaacccaaaatttctttttctaccccactgtactgttagctgtagctattttgtttcttttggataccagggtcaccaccatcttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 28 - DNA, 6795 bp, Zea mays
  source 1..6795: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagaatgtttactatgctcaaaacccaaaatttctttttctaccccactgtactgttagctgtagctattttgtttcttttggataccagggtcacttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 29 - DNA, 6810 bp, Zea mays
  source 1..6810: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggctgtatgtttactatgctcaaaacccaaaatttctttttctaccccactgtactgttagctgtagctattttgtttcttttggataccagggtcaccacatcttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 30 - DNA, 6801 bp, Zea mays
  source 1..6801: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggctgtatgtttactatgctcaaaacccaaaatttctttttctaccccactgtactgttagctgtagctattttgtttcttttggataccagggtcacctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 31 - DNA, 6807 bp, Zea mays
  source 1..6807: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggctgtatgtttactatgctcaaaacccaaaatttctttttctaccccactgtactgttagctgtagctattttgtttcttttggataccagggtcaccacctcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 32 - DNA, 6804 bp, Zea mays
  source 1..6804: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggctgtatgtttactatgctcaaaacccaaaatttctttttctaccccactgtactgttagctgtagctattttgtttcttttggataccagggtcacttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 33 - DNA, 6805 bp, Zea mays
  source 1..6805: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggctgtatgtttactatgctcaaaacccaaaatttctttttctaccccactgtactgttagctgtagctattttgtttcttttggataccagggtcaccctcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 34 - DNA, 6703 bp, Zea mays
  source 1..6703: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagattcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 35 - DNA, 6699 bp, Zea mays
  source 1..6699: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggcaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 36 - DNA, 6804 bp, Zea mays
  source 1..6804: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggtatgtttactatgctcaaaacccaaaatttctttttctaccccactgtactgttagctgtagctattttgtttcttttggataccagggtcaccacttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 37 - DNA, 6795 bp, Zea mays
  source 1..6795: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggctgtatgtttactatgctcaaaacccaaaatttctttttctaccccactgtactgttagctgtagctattttgtttcttttggataccagctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 38 - DNA, 6804 bp, Zea mays
  source 1..6804: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggctgtatgtttactatgctcaaaacccaaaatttctttttctaccccactgtactgttagctgtagctattttgtttcttttggataccagggtcaccccctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 39 - DNA, 6806 bp, Zea mays
  source 1..6806: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagaaggctgtatgtttactatgctcaaaacccaaaatttctttttctaccccactgtactgttagctgtagctattttgtttcttttggataccagggtcaccattcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 40 - DNA, 6801 bp, Zea mays
  source 1..6801: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagatgtttactatgctcaaaacccaaaatttctttttctaccccactgtactgttagctgtagctattttgtttcttttggataccagggtcaccaccatcttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 41 - DNA, 6796 bp, Zea mays
  source 1..6796: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagatgtttactatgctcaaaacccaaaatttctttttctaccccactgtactgttagctgtagctattttgtttcttttggataccagggtcaccattcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 42 - DNA, 6567 bp, Zea mays
  source 1..6567: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttactatgctcaaaacccaaaatttctttttctaccccactgtactgttagctgtagctattttgtttcttttggataccagggtcacctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 43 - DNA, 6865 bp, Zea mays
  source 1..6865: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagcgacagcaggtaccttgacagcaggttcctctgcttctggtcgagcgacagcaggtaccttgacaatgtttactatgctcaaaacccaaaatttctttttctaccccactgtactgttagctgtagctattttgtttcttttggataccagggtcaccaccttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 44 - DNA, 6574 bp, Zea mays
  source 1..6574: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttactatgctcaaaacccaaaatttctttttctaccccactgtactgttagctgtagctattttgtttcttttggataccagggtcaccaccttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 45 - DNA, 6858 bp, Zea mays
  source 1..6858: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagcgacagcaggtaccttgacagcaggttcctctgcttctggtcgagcgacagcaggtaccttgacaatgtttactatgctcaaaacccaaaatttctttttctaccccactgtactgttagctgtagctattttgtttcttttggataccagggtcacctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 46 - DNA, 6783 bp, Zea mays
  source 1..6783: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagaaaacccaaaatttctttttctaccccactgtactgttagctgtagctattttgtttcttttggataccagggtcaccatcttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 47 - DNA, 6795 bp, Zea mays
  source 1..6795: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagatgtttactatgctcaaaacccaaaatttctttttctaccccactgtactgttagctgtagctattttgtttcttttggataccagggtcgttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 48 - DNA, 6778 bp, Zea mays
  source 1..6778: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagaaaacccaaaatttctttttctaccccactgtactgttagctgtagctattttgtttcttttggataccagggtcgttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 49 - DNA, 6800 bp, Zea mays
  source 1..6800: mol_type = genomic DNA
atgggttggctgaccaagttttttagaggctcaacccacaatatctcggaagagcagtatcacagcaggcctgcagaagacacagcatggaatgaaccctctagctctcctgttgtgactgtaagaatatgccatgcgctactgttaacattttatgtttttgaacaatgctgtgtactggagttcctcgtacatccctacactctagttcaggataaaaggatgcaattctgtttatctagcttaggcaaaggagatgcagttggtgtgagctccaagaaaacttgtcctgttcttgttattgagaaaaacgaaagttctgatgtatacaatggatataatttttctacccaagttacttgaaaggacggtttttattttactccaattgctataaatatataccactatgtcttgtccattaaaattctccttatatggatactaatgttcgcaatgttttgatattggtcacatgaaaacaactttttttgttattgcttgtggtggttatggcaacattgatacttcaatcgctatgtacatatacaccagccaatttctgggcaactagccaactataagcttctaataaggttttagatggctccaagctaccaattgcttcaaaaataccatatgccgttgttataagcttttaagtaataagttgattatcttactgatcacatatcggcatgcaatgtaggacatcctttcagagtttaacaatgaggatattgaccgtgctatagcgctctctctatcggaagaggagcaaagaaaggaaaaggcaataggttggtatgctttcttattaatttatgctgcgacattcatccaggttcacttatgttgcctgtgatatgacattgtcaaggtacttcgtgttttagagaagcacctatgtctcatattaactggtacttaaaatgccctttgaagccaatccacagcatatgatgatagtatgatcatcgaaaatctttgttctaggatagttaacttattgctgttcattgtttttgatcttgtctaattttgatcaagaaagccgttccttttccatctttaagtggaaatagttcattctagatttctatggatttcttaggtaaatggaaacataagattacaaaaggatctactcataaggaaaccaggagttatcttatagttaattgtttcttactgcatccttgcttctatattcttttaaagacggattcaagaatgttttcttcaatttctgtggttaaatgtaacaattactaactaaatattcttatggacttcaaccaagaccatgtaagaaaagtgcatccctgaattgagcattagtgtgttacatagtagtgtattgtgttcaaaattttctctaagactattgttactcagtttacttcatgtctttagtgctgttagttttcttatgaattcatgcagttaatgtgtgacactttctggtttaatttgtttttacttctagataaggatatgcatttggaggaggatgagcaacttgcaagagctatccaggaaagtttgaatgttgagtcaccccctcgcagaaatggcagtgccaatggtggtactatgtatcatccgcctcgcgaaactggcaatgcatatcagccacctcgtgaaaatggcagtgccaatggtggaaatgcatatcacccattaccatttatgttctcatctggattcaggttcatttcaatttggttactaagtttactattatagttttgtgcaatttgagttgatggttatgaaatagtaaataagcatataactgaactaatatttttgtttccagaaaggaagtacaccactcatggaccatacatgctgcacatgttactgctaaaaaaacagcaaatcttatttttgtactattcccaccaaaagcgcctgaatattgatttatgtaattcattccctgaataccactatttgtttattggcttcacgcgaaagggcctctagatgagttggttaagtggcctgagcaccactcctcaggtcctgagttcaaatcctagtgggagcgaatttcagactgaggttaaaaaatgtcactcgctggttcccctggttgcgtgcacacgagatgaattgacctaagagctttcctgatctttctcggtcggggctccgattgagcatcttcttaatataatatcgtgggggcggtcttttccgtactggccgagtttaatggcttcacaaacactgctagtcacaacgatataatgagcctgtttggttcaacttttttctgacgagcttttctaagacctagttgtggggagtctgagtatcgtgaggattacatacagaggaagataaaagggtccatagggtttaggatctagaaagagtcgaattcctcctatcacgactacttggccaattctgtgttcacgttgatttagacaattttcaccaaagtgattctcatagaagcatgctgaaaagctggcgttatgcagtctgcatcaggcatcagcttctgttggctagaatctggttttaagctgaaacaaacggagcaattatgtcttgagtgagaactcgtcccagcagacttttgaatgggtgcggttatttcgtgttaacaaaactacctaggactttataattattttgttacgaaaatatctgtagaaattagagccgaagaggaatcaaactgtacactccacctcccaccaactgagctatgcatttcttcgttgcaaggaacagagaaaaaacgaatataggtgtctccacttttcaacaactcatgtaaagttctggccttgttttctcccactaaccaattacgttataaccacagaacaaacttggttgtcctatcaggaaaattccagctgagtttgcttatgtagtaaagaagctaaacgcatgatggcataattatttagaaaaaaaatcccacgctgctttaggtatacaactgaatttttttatctgatttgtgtagtgtgcccgttattatgccaagttcagtttccttatcttttccccagtcttcaggaaaactgttacaaactgtggtgaaatttatagctcagatgtagaatgaaagcaaatattttaaagtggacggggcttgtggttatgcgataccttatatgtaaaaggatgaagcatgtcctcaaggatgttgtttccttaaatagaagtgccttggacatgtgaaatttccaatgctatcaaaagattaagaaattgtacatatgtgaattttcaaatgctgtcaaataaacaagaaattgtgtatgatttttcacatcgattgtagtaataaagacgctgtaatgctcaagaacacaccaggatgattggcatggaagatatcattttgaccttttttctcttactatttttttatataaactaacttcttttctgcagagcatgcgcaggatgtcacagagagattggtcacgggcgttttctcagttgcatgggagctgtttggcatccggaatgttttcgttgccatgcatgtagtcaacctatatatgactatgaggtaacttttttgtttactttttagcagtttctttcaatgtactgaccttaaagccatgctgcagttctccatgtccgggaatcatccataccataaaacttgctacaaggagcagtttcacccgaaatgtgatgtctgcaagcagttcgtaaggatcactttatttaccctgaccgatctgtttatgctataagttttttctgttgaacattttacttgcctgtttgtgctaataaaatctatctccaatatcttggagaatgtgctttctctacatttttcatgattgacatgtttactcttgctaatatctgattacttcttttcagattcctacaaatatgaatggccttattgaatatagggcacatcctttctgggtacaaaaatactgtccttcacatgaaatggatggtactccgaggtgctgtagttgtgaaagaatggaggtaggccagttagcatgtttttcttaatttggtcttgtgggcatatttcatggttagaagagagcctctgtgtgtgaaatttggatcatttcattccaacatggtttgttcacttaccagccaagggaatcaaaatatgtattactggatgatggtcgtaagctctgcctggagtgccttgattccgcagtgatggatacaaacgactgccagcctttgtatcttgaaattcaggaattttacgaaggactgaatatgaaagtggaacagcaggttcctctgcttctggtcgagcgacaagctttgaatgaagccatggaaggagagatgtttactatgctcaaaacccaaaatttctttttctaccccactgtactgttagctgtagctattttgtttcttttggataccagggtcaccatcttcctgaaacaagaggactatgcttatcagaagaacagactgttagcacggtgagagtttttccaaacaccaattggattattctttccttatttaattcttgttctgttccaattttgttcttctgctgcattctgaatgagactgatgttttcagatactgagaccaagaatggctggaaacaaaattatgggaatgataacagagccatataggctgacacggcgatgtgaagtgactgcaattcttattctgtatggtctcccaaggtgaaatgtcaaccttttgttttgtataatgtcataatcagtacctcctgaccgagacaatctgctgccccagctttaatctctacgtgcacttttgccctctttcttttcactatttgccattgcccctgctttggccaagtcaacacacagttgcccctggttttgccacttttttgcaatgaaagtaggcaaaaccatagggatcagtgtgtttgagttggttaaaaaaaactcagtgggagagaaagagtgggagagaaagactgccctcacggtattatattaagaagagaccgaaatatggtcacgcccgagaaaatccctgaaccctggcttcccatcactagcgggccgtcactgcccactctgcaacgccccagccggagggtggcgcgcaggctgtaacctgggagcgggcggggcacaacgaggaaattttttttaaccaagctagaaattcgctcccaagggggattgaacccaggacctggaggtgctactcggaagccttaaccattacgctggaggcagcaagggcaatggtgcagagttaaagaaaaggggcacgcgtgcagtttagaggtcagagttgaagaaaatgggacaactaggtcagcaaagtggtttctttcagtctcttcagatgttttttccttctttgtgccctccaaaaagttgcatgagaaccgagctagtgcttaggtcgataagcaaggtgggctatgatgggtgtgtgtaggggtggtaatgggctctatgttttagattataaaatttaaggatcggatcattattaggatcggactctatttttattcatttttgaactaaaaattaattaagagattaaacaaatcgtgaagaaacatttgagttgtgatccattaccacccttaggtgtgtgtgagtagcgtgtgtgtgtgttcacatatgaaccaatgctacagctgtacccagatgcgaggcttcaaaaaaaagttgcatggaattggagatgtctggagtatgaactgactgcagacttcacgaggaaaatagtttactgttgtccatcgtcacaaatatcttctcctataccatgttgttttgtttggttctacattgttgaatcattaaggatgctcaaatttgtttgtatagacttttatggtgttttgttcttgcagattgttgacaggttcaattttagctcatgagatgatgcatgcatggttgcggcttaaaggtgatacctagttcttgtgttcttcctcggcccaaaataagggagacctatttagatctgacacagcaactaagggtgtgtttggttgtgggacagctaggacgggatcgtcccctgacgtcctctctcgtccctccaattttgagggacaacagtcaacaggggacaacacaggatagtactgtcccaacccttgacccagaaccaaacaacctaatttgagggatcgtcccgtcccatcccgtcctgtcattgcaaccaaacgcaccctaaactaatgttttagttctaggctagcaaggacattaaattgttttcaagactgaacaagtttttattagccaattctagtgaactggtggggctgccatgtaaattgttagaactcagttttggtgtcacagataattactaactagaaataagaacacaaccaatgctaatttgtcggtgaagaaaatcttgcattcatttggcaattgccataataatttgcgtacacatcctatctacttgaaactgttacaaggacatttctgctaatgaatatgtcatcacaggataccggacactgagcccagacgtagaagagggcatttgccaagttcttgctcacatgtggatcgagtcagagatcatggccggatcaggcagtagtgccgcatcgtcatcctcaggatcttcgtcatccacatcatcgaaaaagggcgggcggtcccagtttgagcacaggcttggggactttttcaagcatcagatcgagacggatacttccatggcctacggagatggtttcagaactgggaatcgagccgttcttcattacggcttgaagcgcacccttgagcatatccggctaactgggacctttccattttga

SEQ ID NO: 50 - DNA, 78 bp, Zea mays
  source 1..78: mol_type = genomic DNA
ctgttagctgtagctattttgtttcttttggataccagggtcaccattcctgaaacaagaggactatgcttatcagaa

SEQ ID NO: 51 - DNA, 73 bp, Zea mays
  source 1..73: mol_type = genomic DNA
acaagctttgaatgaagccatggaaggagatgtttactatgctcaaaacccaaaatttctttttctaccccac

SEQ ID NO: 52 - DNA, 68 bp, Zea mays
  source 1..68: mol_type = genomic DNA
aatttggatcatttcattccaacatggtttactatgctcaaaacccaaaatttctttttctaccccac

SEQ ID NO: 53 - DNA, 140 bp, Zea mays
  source 1..140: mol_type = genomic DNA
acaagctttgaatgaagccatggaaggagagcgacagcaggtaccttgacagcaggttcctctgcttctggtcgagcgacagcaggtaccttgacaatgtttactatgctcaaaacccaaaatttctttttctaccccac

SEQ ID NO: 54 - DNA, 80 bp, Zea mays
  source 1..80: mol_type = genomic DNA
ctgttagctgtagctattttgtttcttttggataccagggtcaccaccttcctgaaacaagaggactatgcttatcagaa

SEQ ID NO: 55 - DNA, 73 bp, Zea mays
  source 1..73: mol_type = genomic DNA
ctgttagctgtagctattttgtttcttttggataccagggtcacctgaaacaagaggactatgcttatcagaa

SEQ ID NO: 56 - DNA, 58 bp, Zea mays
  source 1..58: mol_type = genomic DNA
acaagctttgaatgaagccatggaaggagaaaacccaaaatttctttttctaccccac

SEQ ID NO: 57 - DNA, 75 bp, Zea mays
  source 1..75: mol_type = genomic DNA
acaagctttgaatgaagccatggaaggagagatgtttactatgctcaaaacccaaaatttctttttctaccccac

SEQ ID NO: 58 - DNA, 75 bp, Zea mays
  source 1..75: mol_type = genomic DNA
ctgttagctgtagctattttgtttcttttggataccagggtcgttcctgaaacaagaggactatgcttatcagaa

SEQ ID NO: 59 - DNA, 80 bp, Zea mays
  source 1..80: mol_type = genomic DNA
ctgttagctgtagctattttgtttcttttggataccagggtcaccatcttcctgaaacaagaggactatgcttatcagaa

(19) United States
(12) Patent Application Publication
(10) Pub. No.: US 2023/0354762 A1
(43) Pub. Date: Nov. 9, 2023

(54) COMPOSITIONS AND METHODS FOR ENHANCING CORN TRAITS AND YIELD USING GENOME EDITING

(71) Applicant: Monsanto Technology LLC, St. Louis, MO (US)

(72) Inventors: Steven Beach, St. Louis, MO (US); Sivalinganna Manjunath, Chesterfield, MO (US); Linda Rymarquis, High Ridge, MO (US); Thomas L. Slewinski, Chesterfield, MO (US); Xiaoyun Wu, Chesterfield, MO (US)

(21) Appl. No.: 18/190,739

(22) Filed: Mar. 27, 2023

Related U.S. Application Data
(60) Provisional application No. 63/324,985, filed on Mar. 29, 2022.

(51) Int. Cl. A01H 6/46; C12N 15/82; A01H 5/10; C12N 9/22

(52) U.S. Cl. CPC A01H 6/4684; A01H 5/10; C12N 9/22; C12N 15/8262; C12N 2310/20

(57) ABSTRACT

Provided are compositions and methods for reducing, disrupting, or altering ZmDA1 activity in corn plants. Methods and compositions are also provided for producing modifications in the ZmDA1 gene through mutagenesis and/or editing. Modified plant cells and plants having a modification in the ZmDA1 gene are further provided comprising improved characteristics, such as increased yield.

### CROSS REFERENCE TO RELATED APPLICATIONS

This application claims the priority of U.S. Provisional Appl. Ser. No. 63/324,985, filed Mar. 29, 2022, the entire disclosure of which is incorporated herein by reference.

### INCORPORATION OF SEQUENCE LISTING

A sequence listing containing the file named “MONS524US_ST26.xml” which is 321 kilobytes (measured in MS-Windows®) and created on Mar. 14, 2023, and comprises 59 sequences, is incorporated herein by reference in its entirety.

### FIELD OF THE INVENTION

The present disclosure relates to the field of agricultural biotechnology, and to methods and compositions for genome editing in plants. In particular, the invention relates to methods and compositions for producing corn plants exhibiting increased yield and improved kernel characteristics.

### BACKGROUND OF THE INVENTION

Precise genome editing technologies are powerful tools for engineering gene expression and modulating protein function and have the potential to improve important agricultural traits. A continuing need exists in the art to develop novel compositions and methods to effectively and efficiently edit the corn plant genome in order to increase yield and achieve other agronomic benefits.

### SUMMARY

Provided herein are modified corn plants, corn plant seeds, corn plant parts, or corn plant cells, comprising a genomic modification that reduces or disrupts the activity of ZmDA1, as compared to the activity of ZmDA1 in an otherwise identical corn plant, corn plant seed, corn plant part, or corn plant cell that lacks the modification. In some embodiments, the modification is present in at least one allele of an endogenous ZmDA1 gene. In particular embodiments, the endogenous ZmDA1 gene encodes a protein having at least 70%, at least 75%, at least 80%, at least 85%, at least 90%, at least 95%, at least 96%, at least 97%, at least 98%, at least 99%, at least 99.5%, or 100% sequence identity to SEQ ID NO:2. In other embodiments, the modification is in a transcribable region of the ZmDA1 gene, non-limiting examples of which include a region of said ZmDA1 gene comprising a coding sequence for a LIM domain, a region of said ZmDA1 gene comprising a coding sequence for a LIM-like domain, a region of said ZmDA1 gene comprising a coding sequence for a C-terminal DA1-like domain, an exon region, an intron region, and combinations of any thereof. In some embodiments, the plant, plant seed, plant part, or plant cell is heterozygous for the modification, and in other embodiments, the plant, plant seed, plant part, or plant cell is homozygous for the modification. In other embodiments, the plant, plant seed, plant part, or plant cell comprises a polynucleotide sequence selected from the group consisting of SEQ ID NOs:11-49. In certain embodiments, the modified plant exhibits increased yield, grain yield estimate per plant, grain yield estimate, or combinations of any thereof, as compared to an otherwise identical plant that lacks the modification. In other embodiments, the plant, plant seed, plant part, or plant cell is defined as comprising a first modification in a first allele of the ZmDA1 gene and a second modification in a second allele of the ZmDA1 gene, the first modification and the second modification being different from one another.

A modified corn plant, corn plant seed, corn plant part, or corn plant cell provided herein may, in certain embodiments, comprise a modification, wherein the modification comprises a deletion, an insertion, a substitution, an inversion, a duplication, or any combination thereof. In some embodiments, for example, the modification is located at about 2369 nucleotides or more upstream from the 3′ end of reference sequence SEQ ID NO:3; or is located at about 3650 nucleotides or more downstream from the 5′ end of reference sequence SEQ ID NO:3. Also provided herein are modified plants or seeds, plant parts, cells thereof, comprising a modification that disrupts or alters the activity of ZmDA1, as compared to the activity of ZmDA1 in an otherwise identical plant, plant seed, plant part, or plant cell that lacks the modification. In some embodiments, the modification confers an altered phenotype to the plant, as compared to the phenotype of an otherwise identical plant that lacks the modification. The plant, plant seed, plant part, or plant cell can also comprise, for example, a modification in at least one allele of the ZmDA1 gene, wherein the modification is selected from the group consisting of: a 115 base pair deletion wherein the resulting nucleotide sequence is SEQ ID NO:11; a 10 base pair deletion and a 4 base pair deletion wherein the resulting nucleotide sequence is SEQ ID NO:12; a 7 base pair deletion and a 6 base pair deletion wherein the resulting nucleotide sequence is SEQ ID NO:13; an 8 base pair modification and an 11 base pair deletion wherein the resulting nucleotide sequence is SEQ ID NO:14; a 10 base pair deletion and a 6 base pair deletion wherein the resulting nucleotide sequence is SEQ ID NO:15; a 113 base pair deletion wherein the resulting nucleotide sequence is SEQ ID NO:16; a first 6 base pair deletion and a second 6 base pair deletion wherein the resulting nucleotide sequence is SEQ ID NO:17; a 29 base pair deletion and a 3 base pair deletion wherein the resulting nucleotide sequence is SEQ ID NO:18; a 111 base pair modification wherein the resulting nucleotide sequence is SEQ ID NO:19; a 10 base pair deletion and a 3 base pair deletion wherein the resulting nucleotide sequence is SEQ ID NO:20; a 10 base pair deletion and a 9 base pair deletion wherein the resulting nucleotide sequence is SEQ ID NO:21; a 108 base pair deletion wherein the resulting nucleotide sequence is SEQ ID NO:22; a 12 base pair deletion and a 11 base pair deletion wherein the resulting nucleotide sequence is SEQ ID NO:23; an 18 base pair deletion and a 7 base pair deletion wherein the resulting nucleotide sequence is SEQ ID NO:24; a 6 base pair deletion and a 4 base pair deletion wherein the resulting nucleotide sequence is SEQ ID NO:25; a 624 base pair deletion and a 24 base pair deletion wherein the resulting nucleotide sequence is SEQ ID NO:26; an 8 base pair deletion wherein the resulting nucleotide sequence is SEQ ID NO:27; a 9 base pair deletion and a 7 base pair deletion wherein the resulting nucleotide sequence is SEQ ID NO:28; a 1 base pair deletion wherein the resulting nucleotide sequence is SEQ ID NO:29; a 10 base pair deletion wherein the resulting nucleotide sequence is SEQ ID NO:30; a 4 base pair deletion wherein the resulting nucleotide sequence is SEQ ID NO:31; a 7 base pair deletion wherein the resulting nucleotide sequence is SEQ ID NO:32; a 7 base pair modification wherein the resulting nucleotide sequence is SEQ ID NO:33; a 108 base pair deletion wherein the resulting nucleotide sequence is SEQ ID NO:34; a 112 base pair deletion wherein the resulting nucleotide sequence is SEQ ID NO:35; a 3 base pair deletion and a 4 base pair deletion wherein the resulting nucleotide sequence is SEQ ID NO:36; a 16 base pair deletion wherein the resulting nucleotide sequence is SEQ ID NO:37; an 8 base pair modification wherein the resulting nucleotide sequence is SEQ ID NO:38; a 5 base pair deletion wherein the resulting nucleotide sequence is SEQ ID NO:39; a 10 base pair deletion wherein the resulting nucleotide sequence is SEQ ID NO:40; a 5 base pair deletion and a 10 base pair deletion wherein the resulting nucleotide sequence is SEQ ID NO:41; a 234 base pair deletion and a 10 base pair deletion wherein the resulting nucleotide sequence is SEQ ID NO:42; a 7 base pair deletion, a 64 base pair insertion, and a 3 base pair deletion wherein the resulting nucleotide sequence is SEQ ID NO:43; a 234 base pair deletion and a 3 base pair deletion wherein the resulting nucleotide sequence is SEQ ID NO:44; a 7 base pair deletion, a 64 base pair insertion, and a 10 base pair deletion wherein the resulting nucleotide sequence is SEQ ID NO:45; a 25 base pair deletion and a 3 base pair deletion wherein the resulting nucleotide sequence is SEQ ID NO:46; an 8 base pair deletion and a 9 base pair modification wherein the resulting nucleotide sequence is SEQ ID NO:47; a 25 base pair deletion and a 9 base pair modification wherein the resulting nucleotide sequence is SEQ ID NO:48; an 8 base pair deletion and a 3 base pair deletion wherein the resulting nucleotide sequence is SEQ ID NO:49; and combinations of any thereof. In certain embodiments, the modification is comprised within a genomic region from nucleotide position 3434 to nucleotide position 6811 with reference to sequence SEQ ID NO:3. In other embodiments, the modification is comprised within a genomic region from about nucleotide position 3650 to about nucleotide position 4437 of reference sequence SEQ ID NO:3. In still further embodiments, the modification is comprised within a genomic region from about nucleotide position 3650 to about nucleotide position 4443 of reference sequence SEQ ID NO:3. In some embodiments, the modification comprises a deletion of at least about 1, at least about 3, at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 125, at least about 150, at least 200, at least 300, at least 600 or at least 650 consecutive nucleotides. In some embodiments, the modification comprising the deletion may also include nucleotide substitutions and/or nucleotide insertions of at least 1, at least 2, at least 3, at least 4, at least 6, at least 8, at least 10, or at least 20 consecutive nucleotides around the deletion. A plant, plant seed, plant part, or plant cell provided herein can also comprise, for example, a chromosomal sequence in the ZmDA1 gene that has at least 70%, at least 75%, at least 80%, at least 85%, at least 90%, at least 95%, at least 96%, at least 97%, at least 98%, at least 99%, at least 99.5%, or 100% sequence identity to SEQ ID NO:3 in the regions outside of the deletion, the insertion, the substitution, the inversion, or the duplication. In certain embodiments, the modification alters peptidase activity of ZmDA1, as compared to the activity of ZmDA1 in an otherwise identical plant that lacks the modification. In certain embodiments, the altered phenotype, for example, comprises an increase in number of kernels per ear, number of kernels per longitudinal row of ear, ear area, ear length, yield, grain yield estimate per plant, grain yield estimate, or combinations of any thereof, as compared to the phenotype of an otherwise identical plant that lacks the modification. In some embodiments, the plant, plant seed, plant part, or plant cell comprises a polynucleotide sequence selected from the group consisting of SEQ ID NOs:11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, and 49. In other embodiments, the modification is comprised within a genomic region from nucleotide position 4325 to nucleotide position 4433 with reference to sequence SEQ ID NO:3; the modification is comprised within a genomic region from nucleotide position 4107 to nucleotide position 4437 of reference sequence SEQ ID NO:3; or the modification is comprised within a genomic region from nucleotide position 4326 to nucleotide position 4433 of reference sequence SEQ ID NO:3.

In certain embodiments, a polynucleotide is provided comprising a sequence selected from the group consisting of SEQ ID NOs: 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, and 49. In specific embodiments, the polynucleotide sequence is a modified endogenous ZmDA1 gene..

Further disclosed herein is a method for producing a corn plant comprising a modified ZmDA1 gene, the method comprising: a) introducing a modification into at least one target site in an endogenous ZmDA1 gene of a corn plant cell that reduces or disrupts the activity of ZmDA1; b) identifying and selecting one or more corn plant cells of step (a) comprising said modification in said ZmDA1 gene; and c) regenerating at least a first plant from said one or more cells selected in step (b) or a descendent thereof comprising said modification. In some embodiments, the target site is located in a coding or non-coding region of an endogenous ZmDA1 gene. In other embodiments, the modification, for example, is in a region of said ZmDA1 gene comprising a coding sequence for a LIM domain, a region of said ZmDA1 gene comprising a coding sequence for a LIM-like domain, a region of said ZmDA1 gene comprising a coding sequence for a C-terminal DA1-like domain, an exon region, an intron region, and combinations of any thereof. In still further embodiments, the modification is facilitated by the presence of at least one site-specific genome modification enzyme in said plant cell. Non-limiting examples of such an enzyme include an RNA-guided nuclease, a zinc-finger nuclease, a meganuclease, a TALE-nuclease, a recombinase, a transposase, and combinations of any thereof. Examples of RNA-guided nucleases include a Cas nuclease, a Cpf1 nuclease, or a variant of either thereof. Some site-specific genome modification enzymes that could find use in accordance with the disclosure create at least one strand break at the target site. The methods disclosed herein may be used, for example, to produce any modification in accordance with the disclosure, including a substitution, an insertion, an inversion, a deletion, a duplication, or any combination thereof. In some embodiments, the modification is a deletion, and the deletion comprises a region of at least 1, at least 3, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 300, at least 600, or at least 650 consecutive nucleotides. In some embodiments, the modification comprising the deletion may also include nucleotide substitutions and/or nucleotide insertions of at least 1, at least 2, at least 3, at least 4, at least 6, at least 8, at least 10, or at least 20 consecutive nucleotides around the deletion.

Also provided herein is a method for producing a hybrid corn plant comprising a modified ZmDA1 gene, the method comprising crossing a corn plant comprising a modified ZmDA1 gene with a second, non-isogenic corn plant to produce a F1 hybrid corn plant, wherein the modified ZmDA1 gene confers an altered phenotype to the hybrid corn plant as compared to the phenotype of an otherwise isogenic hybrid corn plant that lacks the modification. In some embodiments, the second, non-isogenic corn plant lacks a modified ZmDA1 gene.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 ZmDA1 gene sequence (SEQ ID NO:3) and protein sequence of ZmDA1. Panel A schematically shows the gene sequence including marked exons (black arrows) and the 785 bp effective targeting region by gRNAs (light gray box) of the ZmDA1 gene. Panels B and C show the protein sequence of ZmDA2 with the UIM domains, LIM domain, LIM-like domain, and C-terminal DA1-like domain annotated (gray arrow) and including the region modified by editing (light gray box).

FIG. 2 shows an alignment of unique, non-redundant sequence edits in ZmDA1 (DA1_edit1 - DA1_edit9; SEQ ID NOs: 11-19, respectively) as compared to DA1_WT (SEQ ID NO:3). Asterisks (*) indicate positions in which all edited sequences maintain the same base as the wild-type (WT), lacking either base deletion or base substitution/insertion. Dashes (-) indicate base deletions compared to WT. Sequence between segments marked 3660 and 4320 (first and second panel) is excluded due to absence of edits in the region. Additionally, consensus sequences upstream of position 3600 and downstream of position 4440 with reference to SEQ ID NO:3 are excluded due to absence of gene edits in these regions. If present, nucleotide insertions and/or substitutions around deleted regions are shown. Letter case of base is arbitrary.

FIG. 3 shows changes in yield-related traits resulting from ZmDA1 gene edited plants tested (DA1_edit1 - DA1_edit9). Results are shown as percent difference (delta) between edited plants and control plants. Dark gray bars represent significant increase or decrease at P value less than 0.2. Light gray bars represent increase or decrease in yield related trait at P value 0.2 and above. Ear size related traits were measured through imaging analysis.

FIG. 4shows an alignment of sequence edits in ZmDA1 (DA1_edit10 through DA1_edit28; SEQ ID NOs: 20-38, respectively) as compared to the genomic sequence for the WT ZmDA1 gene (SEQ ID NO:3). The top panel uses grey arrows to designate the different functional regions of the ZmDA1 gene. Middle panel uses black arrows to denote the exons of the ZmDA1 gene. The bottom panel shows the alignment of the sequences in the edited regions between 3711 bp and 4480 bp. “DA1_WT” indicates the unedited WT ZmDA1 gene (SEQ ID NO:3). Dashes (-) indicate base deletions compared to WT. Consensus sequences upstream and downstream are excluded due to absence of edits in these regions. Intervening white boxes containing a number of base pairs indicate an uninterrupted region of that length in base pairs in which either the sequence maintained consensus to SEQ ID NO:3 (those ending in a base letter on the left of the box and beginning with a base letter on the right of the same box) or the sequence had a large deletion compared to SEQ ID NO:3 (those ending in a (-) on the left of the box and beginning with a (-) on the right of the same box).

FIG. 5shows an alignment of detected sequence modifications in ZmDA1 (DA1_edit29-1 through DA1₋edit31-4; SEQ ID NOs: 50-59, respectively) as compared to the genomic sequence for the WT ZmDA1 gene (SEQ ID NO:3). DA1_edit29-1 through DA1₋edit31-4 are schematically visualized by the amplicon sequences identifying each modification. The top panel uses grey arrows to designate the different functional regions of the ZmDA1 gene. The middle panel uses black arrows to denote the exons of the ZmDA1 gene. The bottom panel shows the alignment of the amplicons in the edited regions 4103 bp and 4462 bp of SEQ ID NO:3. “DA1_WT” indicates the unedited WT ZmDA1 gene (SEQ ID NO:3). Dashes (-) indicate base deletions compared to WT or a gap in the multiple sequence alignment (as in the DA1_WT, DA1_Edit29-1, DA1_Edit29-2, DA1_Edit30-1, DA1_Edit30-3, DA1_Edit30-4, DA1_Edit31-1, DA1_Edit31-2, DA1_Edit31-3, and DA1_Edit31-4) due to the presence of a 64 bp insertion in DA1_Edit30-2. Blank space indicates sequence outside of the amplified amplicon region. Consensus sequences upstream and downstream may be excluded due to absence of edits in these regions. Intervening white boxes containing a number of base pairs indicate a region of that length in base pairs in which either the sequence maintained consensus to SEQ ID NO:3 (those ending in a base letter on the left of the box and beginning with a base letter on the right of the same box) or the sequence had a large deletion compared to SEQ ID NO:3 (those ending in a (-) on the left of the box and beginning with a (-) on the right of the same box), or the amplicon ends in that absent region. The sequence of each amplicon is as in SEQ ID NOs: 50-59.

### BRIEF DESCRIPTION OF THE SEQUENCES

SEQ ID NO:1 is the polynucleotide coding sequence of the Zea mays DA1 (ZmDA1) gene.

SEQ ID NO:2 is the amino acid sequence for ZmDA1 (encoded by SEQ ID NO:1).

SEQ ID NO:3 is the polynucleotide sequence for the ZmDA1 gene, including the introns and exons, but excluding the 5′ and 3′ untranslated regions (UTRs).

SEQ ID NO:4 is the polynucleotide sequence of a common scaffold compatible with the Cpf1 gene.

SEQ ID NO:5 is the polynucleotide sequence of a Zea mays polyubiquitin promoter.

SEQ ID NO:6 is the polynucleotide sequence encoding a Lachnospiraceae bacterium Cpf1 RNA-guided endonuclease enzyme, codon-optimized for corn.

SEQ ID NO:7 is the polynucleotide sequence for a nuclear localization signal from Solanum lycopersicum.

SEQ ID NOs:8-10 are polynucleotide sequences for the spacer sequences in the guide RNAs (gRNAs) used for editing of the transcribable region of the ZmDA1 gene.

SEQ ID NOs:11-19 are polynucleotide sequences for alleles of the ZmDA1 gene having various deletions as well as nucleotide base substitutions or insertions around the deleted region as compared to SEQ ID NO:3.

SEQ ID NOs:20-38 are polynucleotide sequences for alleles of the ZmDA1 gene in edited homozygous R1 plants having various segmental modifications as compared to SEQ ID NO:3.

SEQ ID NOs:39-49 are polynucleotide sequences for potential homozygous alleles of the ZmDA1 gene obtainable from selfing edited R₀ plants having various segmental modifications as compared to SEQ ID NO:3.

SEQ ID NOs:50-59 are polynucleotide sequences for individual edits detected in edited R₀ plants having various segmental modifications of the ZmDA1 gene as compared to SEQ ID NO:3.

### DETAILED DESCRIPTION

Corn, Zea mays, is a valuable field crop. Thus, a goal of plant breeders is to develop high-yielding corn varieties to maximize the amount of grain produced on arable land, and to supply food for both animals and humans. The majority of commercial corn is produced using hybrid seed. Cultivation of hybrid corn has significant benefits, e.g., it is well documented that hybrid yields are significantly greater, and fields planted with hybrid varieties are genetically uniform. Presently, North American farmers plant tens of millions of acres of corn and there are extensive national and international commercial corn breeding programs. A continuing goal of these corn breeding programs is to develop corn hybrid varieties that have one or more desirable characteristics such as increased yield.

In one example, corn yield may be improved by increasing important yield determinants, e.g., kernel weight and kernel number. Such kernel characteristics are especially important agronomic traits, as they can directly affect yield potential. The development of kernels, i.e., corn seed, is controlled by multiple factors. In part, kernel development is influenced by the ubiquitin pathway gene ZmDA1. Specifically, studies have shown that the ZmDA1 gene and other ubiquitin pathway genes play important roles in regulating cell proliferation and organ size. However, there is a continuing need for discovery and development of new strategies for increasing agronomic performance, especially those strategies that can be directly implemented into the development of hybrid corn varieties having one or more desirable characteristics.

The present disclosure represents a significant advance in the art in that it provides engineered alleles conferring beneficial phenotypes in corn, as well as methods for the production thereof, thereby offering improvements in key traits that lead to increased productivity per plant and plot. The methods and compositions disclosed herein offer the opportunity to create diversity that cannot be achieved from conventional plant breeding or random mutagenesis. Accordingly, provided herein are methods and compositions for reducing, disrupting, or altering the activity of ZmDA1 in corn that may be used to achieve beneficial results, including, e.g., an increase in number of kernels per ear, number of kernels per longitudinal row of ear (i.e., kernel rank), ear size related traits (e.g., ear area, ear length, and ear diameter), single kernel weight, kernel row number, yield, grain yield estimate per plant, grain yield estimate, or combinations of any thereof. Moreover, the ability to produce these desirable characteristics in corn plants that are homozygous, or heterozygous, for the engineered allele offers unique benefits to corn breeders.

To produce such corn plants, the present disclosure provides, in certain embodiments, methods and compositions for the creation of novel alleles at the ZmDA1 locus via editing of the ZmDA1 gene. For example, a transcribable region of the ZMDA1 gene was modified as disclosed herein by use of engineered guide RNAs. For example, regions of the ZmDA1 gene comprising a coding sequence for a LIM domain, a LIM-like domain, or a C-terminal DA1-like domain were targeted for mutagenesis. It was shown that a series of edited alleles at the ZmDA1 locus could be generated, including modifications from 1 to 624 bp. Representative edited individuals harboring a series of modifications from 3 to 115 bp were selected and evaluated. Plants homozygous for the edited alleles were produced by self-crossing; and these homozygous plants were crossed with a non-isogenic male corn plant line to produce hybrid plants. As described herein, hybrid plants heterozygous for the edited allele exhibited increases in key yield related traits, e.g., number of kernels per ear, kernel rank, ear area, ear length, yield, grain yield estimate per plant, and grain yield estimate. Edited alleles at the ZmDA1 locus, as exemplified herein, therefore represent a novel mechanism to confer dominant effects in hybrid corn plants resulting in beneficial agronomic characteristics. The present disclosure thus represents a significant advance in the art in that it permits the production of novel engineered alleles in corn that confer beneficial phenotypes with the potential to increase yield.

### I. Genome Editing

The present disclosure provides, in certain embodiments, corn plants, plant parts, plant cells, and seeds produced through genome modification using site-specific integration or genome editing. Genome editing can be used to make one or more edit(s) or mutation(s) at a desired target site in the genome of a plant, such as to change expression and/or activity of one or more genes, or to integrate an insertion sequence or transgene at a desired location in a plant genome. Any site or locus within the genome of a plant may potentially be chosen for making a genomic edit (or gene edit) or site-directed integration of a transgene, construct, or transcribable DNA sequence. As used herein, a “target site” for genome editing or site-directed integration refers to the location of a polynucleotide sequence within a plant genome that is bound and cleaved by a site-specific nuclease to introduce a double-stranded break (DSB) or single-stranded nick into the nucleic acid backbone of the polynucleotide sequence and/or its complementary DNA strand within the plant genome. A target site may comprise, for example, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for an RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further herein). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by any other site-specific nuclease that may not be guided by a non-coding RNA molecule, such as a zinc finger nuclease (ZFN), a transcription activator-like effector nuclease (TALEN), a meganuclease, etc., to introduce a DSB or single-stranded nick into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion, substitution, inversion, or duplication. As used herein, “flanked” when used to describe a target region of a polynucleotide sequence or molecule, refers to two or more target sites of the polynucleotide sequence or molecule surrounding the target region, with one target site on each side of the target region.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system or the CRISPR/Cpf1 system), a TALE (transcription activator-like effector)-endonuclease (TALEN), a recombinase, or a transposase. As used herein, “editing” or “genome editing” refers to generating a targeted mutation, deletion, insertion, substitution, inversion, or duplication of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” may also encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, insertion, substitution, inversion, or duplication, whereas “edits” or “genomic edits” refers to two or more targeted mutation(s), deletion(s), insertion(s), substitution(s), inversion(s), and/or duplication(s), with each “edit” being introduced via a targeted genome editing technique.

According to some embodiments, a site-specific nuclease may be co-delivered with a donor template molecule to serve as a template for making a desired edit, mutation or insertion into the genome at the desired target site through repair of the double strand break (DSB) or nick created by the site-specific nuclease. According to some embodiments, a site-specific nuclease may be co-delivered with a DNA molecule comprising a selectable or screenable marker gene.

A site-specific nuclease provided herein may be selected from the group consisting of a zinc-finger nuclease (ZFN), a TALE-endonuclease (TALEN), a meganuclease, an RNA-guided endonuclease (e.g., Cas9 and Cpf1), a recombinase, a transposase, or any combination thereof. See, e.g., Khandagale et al. (Plant Biotechnol Rep 10:327-343, 2016); and Gaj et al. (Trends Biotechnol. 31(7):397-405, 2013). Zinc finger nucleases (ZFN) are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site but may typically be composed of 3-4 (or more) zinc-fingers. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The amino acids at positions -1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities.

Methods and rules for designing ZFNs for targeting and binding to specific target sequences are known in the art. See, e.g., U.S. Pat. App. Pub. Nos. 2005/0064474, 2009/0117617, and 2012/0142062. The FokInuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site. Because the DNA-binding specificities of zinc finger domains can be re-engineered using one of various methods, customized ZFNs can theoretically be constructed to target nearly any target sequence (e.g., at or near a gene in a plant genome). Publicly available methods for engineering zinc finger domains include Context-dependent Assembly (CoDA), Oligomerized Pool Engineering (OPEN), and Modular Assembly.

Transcription activator-like effectors (TALEs) can be engineered to bind practically any DNA sequence, such as at or near the genomic locus of a gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

TALENs are artificial restriction enzymes generated by fusing the TALE DNA binding domain to a nuclease domain. In some aspects, the nuclease is selected from a group consisting of PvuII, MutH, TevI, FokI, AlwI, MlyI, SbfI, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevIcleavage domains are useful alternatives to FokI and FokIvariants for use with TALEs. PvuII functions as a highly specific cleavage domain when coupled to a TALE (see Yank et al., PLoS One 8:e82539, 2013). MutH is capable of introducing strand-specific nicks in DNA (see Gabsalilow et al., Nucleic Acids Research. 41:e83, 2013). TevI introduces double-stranded breaks in DNA at targeted sites (see Beurdeley et al., Nature Communications 4:1762, 2013).

The relationship between amino acid sequence and DNA recognition of the TALE binding domain allows for designable proteins. Software programs such as DNAWorks can be used to design TALE constructs. Other methods of designing TALE constructs are known to those of skill in the art. See Doyle et al. (Nucleic Acids Research 40: W117-122, 2012); Cermak et al. (Nucleic Acids Research 39:e82, 2011); and tale-nt.cac.cornell.edu/about. In another aspect, a TALEN provided herein is capable of generating a targeted DSB.

A site-specific nuclease may be a meganuclease. Meganucleases, which are commonly identified in microbes, such as the LAGLIDADG family of homing endonucleases, are unique enzymes with high activity and long recognition sequences (> 14 bp) resulting in site-specific digestion of target DNA. Engineered versions of naturally occurring meganucleases typically have extended DNA recognition sequences (for example, 14 to 40 bp). The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

A site-specific nuclease may be an RNA-guided nuclease. In an aspect, the targeted genome editing described herein may comprise the use of an RNA-guided endonuclease. As used herein, an “RNA-guided nuclease” refers to an RNA-guided DNA endonuclease associated with the CRISPR system. According to some embodiments, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (also known as Cas12a, see e.g., Safari, F. et al., Cell Biosci 9:36, 2019), CasX, CasY, and homologs or modified versions of any thereof, as well as Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions of any thereof). According to some embodiments, an RNA-guided endonuclease is a Cas9 or Cpf1 enzyme. According to some embodiments, an RNA-guided endonuclease is a Cpf1 enzyme.

The CRISPR system, in its native context, provide bacteria and archaea with immunity to invading foreign nucleic acids and relies on an RNA-guided endonuclease to cleave the invading DNA or RNA into short sequence fragments and incorporating them into the bacterial CRISPR genomic locus. The incorporated short sequences, referred to as “protospacers”, and flanking direct repeats are transcribed and processed into CRISPR RNAs (crRNAs). These crRNAs hybridize with trans-activating crRNAs (tracrRNAs) to activate the RNA-guided Cas endonuclease to form a ribonucleoprotein (RNP) complex that is guided to a target site. A prerequisite for cleavage of the target site, however, is the presence of a conserved genomic protospacer-adjacent motif sequence recognized by the Cas endonuclease. A “protospacer adjacent motif” (PAM) herein refers to a short nucleotide sequence adjacent to a target sequence (protospacer) that is recognized (targeted) by a guide polynucleotide/Cas endonuclease system described herein. A PAM may be present in the genome immediately adjacent and upstream to the 5′ end of the genomic target site sequence complementary to the targeting sequence of the guide RNA - i.e., immediately downstream (3′) to the sense (+) strand of the genomic target site (relative to the targeting sequence of the guide RNA) as known in the art. See, e.g., Wu et al. (Quant Biol. 2(2):59-70, 2014). The Cas endonuclease may not successfully recognize a target DNA sequence if the target DNA sequence is not followed by a PAM sequence. The sequence and length of a PAM sequence herein can differ depending on the Cas endonuclease used. The PAM sequence can be of any length but is typically 1, 2, 3, 4, 5, 6, 7, 8, 9, 10, 11, 12, 13, 14, 15, 16, 17, 18, 19 or 20 nucleotides long.

CRISPR/Cas9, which is the CRISPR system from Streptococcus pyogenes, was adapted for use in eukaryotes and has been widely used for gene editing in plants. The CRISPR/Cas9 system requires both crRNA and tracrRNA to guide the Cas9 protein to recognize and cleave the target DNA double helix. Cas9 recognizes the genomic PAM sequence 5′-NGG-3′ (where N is any nucleotide) and, when located on the sense (+) strand adjacent to the target site, will create a blunt-end DSB at the target site, specifically the 5′-end of the PAM site. Cas9 has been observed to recognize other PAM sequences, such as 5′-NAG-3′and 5′-NGA-3,’ which may result in cleavage of non-specific DNA sequences. However, the corresponding sequence of the guide RNA (i.e., immediately downstream (3′) to the targeting sequence of the guide RNA) may generally not be complementary to the genomic PAM sequence.

Recently, the CRISPR/Cpf1 system was discovered as an alternative to the CRISPR/Cas9 system for genome editing. While CRISPR/Cpf1 functions in a manner similar to CRISPR/Cas9, it is an even simpler system than CRISPR/Cas9. CRISPR/Cpf1 requires only one crRNA molecule and no tracrRNA to cleave DNA. Cpf1 recognizes the genomic PAM sequence 5′-TTTV-3′ (where V is A, G, or C) or 5′-TTN-3′, depending on the Cpf1 ortholog. See e.g., Alok et al. (Front. Plant Sci. 11:264, 2020). When Cpf1 recognizes the genomic PAM located on the sense (+) strand adjacent to the target site, it will generate a staggered DSB with a 4 or 5-nt 5′ overhang at the target site, specifically the 3′-end of the PAM site.

The RNA-guided nuclease may be delivered as a protein with or without a guide RNA, or the guide RNA may be complexed with the RNA-guided nuclease enzyme and delivered as a ribonucleoprotein (RNP).

For RNA-guided endonucleases, a guide RNA molecule may be further provided to direct the endonuclease to a target site in the genome of the plant via base-pairing or hybridization to cause a DSB or nick at or near the target site. The guide RNA may be transformed or introduced into a plant cell or tissue as a gRNA molecule, or as a recombinant DNA molecule, construct or vector comprising a transcribable DNA sequence encoding the guide RNA operably linked to a promoter. As understood in the art, a guide RNA may comprise, for example, a CRISPR RNA (crRNA), a single-chain guide RNA (sgRNA), or any other RNA molecule that may guide or direct an endonuclease to a specific target site in the genome. A prototypical CRISPR associated protein, Cas9 from S. pyogenes, naturally binds two RNAs, a CRISPR RNA (crRNA) guide and a transacting CRISPR RNA (tracrRNA), to assemble a CRISPR ribonucleoprotein (crRNP). A “single-chain guide RNA” (or “sgRNA”) is an RNA molecule comprising a crRNA covalently linked to a tracrRNA by a linker sequence, which may be expressed as a single RNA transcript or molecule. The guide RNA comprises a guide or targeting sequence (also referred to herein as a “spacer sequence”) that is identical or complementary to a target site within the plant genome, such as at or near a gene. The guide RNA is typically a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site. According to some embodiments, a guide RNA comprising a polynucleotide sequence selected from the group consisting of SEQ ID NOs:8, 9, and 10 is provided herein.

In addition to the guide sequence, a guide RNA may further comprise one or more other structural or scaffold sequence(s), which may bind or interact with an RNA-guided endonuclease. Such scaffold or structural sequences may further interact with other RNA molecules (e.g., tracrRNA). Methods and techniques for designing targeting constructs and guide RNAs for genome editing and site-directed integration at a target site within the genome of a plant using an RNA-guided endonuclease are known in the art.

As mentioned above, a target gene for genome editing may be the Zea mays DA1 (ZmDA1) gene. For modification of the ZmDA1 gene through genome editing, an RNA-guided endonuclease may be targeted to a transcribable DNA sequence (i.e., a transcribable region) of said gene, such as a region of said ZmDA1 gene comprising a coding sequence for a LIM domain, a region of said ZmDA1 gene comprising a coding sequence for a LIM-like domain, a region of said ZmDA1 gene comprising a coding sequence for a C-terminal DA1-like domain, an exon region, an intron region, or a combination thereof. For example, in certain embodiments a transcribable DNA sequence targeted for genome editing may comprise an exon/intron boundary or may be in close proximity to an exon/intron boundary. If the resulting modification spans an exon/intron boundary, the modification may be referred to as a modification in an exon region and an intron region. For genetic modification of the ZmDA1 gene, a guide RNA may be used, which comprises a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO:3 or a sequence complementary thereto (e.g., 10, 11, 12, 13, 14, 15, 16, 17, 18, 19, 20, 21, 22, 23, 24, 25 or more consecutive nucleotides of SEQ ID NO:3 or a sequence complementary thereto), although alternative splicing and different exon/intron boundaries may occur. As used herein, the term “consecutive” in reference to a polynucleotide or protein sequence means without deletions or gaps in the sequence.

As used herein, with respective to a given sequence, a “complement”, a “complementary sequence” and a “reverse complement” are used interchangeably. All three terms refer to the inversely complementary sequence of a nucleotide sequence, i.e., to a sequence complementary to a given sequence in reverse order of the nucleotides.

As used herein, the term “antisense” refers to DNA or RNA sequences that are complementary to a specific DNA or RNA sequence. Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as an RNA molecule transcribed from the gene or locus (with the exception of uracil in RNA and thymine in DNA).

A protospacer-adjacent motif (PAM) may be present in the genome immediately adjacent and upstream to the 5′ end of the genomic target site sequence complementary to the targeting sequence of the guide RNA - i.e., immediately downstream (3′) to the sense (+) strand of the genomic target site (relative to the targeting sequence of the guide RNA) as known in the art. See, e.g., Wu et al. (Quant Biol. 2(2):59-70, 2014). However, the corresponding sequence of the guide RNA (i.e., immediately downstream (3′) to the targeting sequence of the guide RNA) may generally not be complementary to the genomic PAM sequence.

In some embodiments, a site-specific nuclease is a recombinase. Non-limiting examples of recombinases that may be used include a serine recombinase attached to a DNA recognition motif, a tyrosine recombinase attached to a DNA recognition motif, or any recombinase enzyme known in the art attached to a DNA recognition motif. In certain embodiments, the site-specific nuclease is a recombinase or transposase, which may be a DNA transposase or recombinase attached or fused to a DNA binding domain. Non-limiting examples of recombinases include a tyrosine recombinase selected from the group consisting of a Cre recombinase, a Gin recombinase, a Flp recombinase, and a Tnp1 recombinase attached to a DNA recognition motif provided herein. In one aspect of the present disclosure, a Cre recombinase or a Gin recombinase provided herein is tethered to a zinc-finger DNA-binding domain, a TALE DNA-binding domain, or a Cas9 nuclease. In another aspect, a serine recombinase selected from the group consisting of a PhiC31 integrase, an R4 integrase, and a TP-901 integrase may be attached to a DNA recognition motif provided herein. In yet another aspect, a DNA transposase selected from the group consisting of a TALE-piggyBac and TALE-Mutator may be attached to a DNA binding domain provided herein.

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site at or near the genomic locus of an endogenous gene of a plant to create a DSB or nick at such a genomic locus. The DSB or nick created by the non-RNA-guided site-specific nuclease may lead to knockdown of gene expression, or a change in the activity of the protein encoded by the endogenous gene, via repair of the DSB or nick, which may result in a mutation or insertion of a sequence at the site of the DSB or nick through cellular repair mechanisms. Such cellular repair mechanism may be guided by a donor template molecule.

As used herein, a “donor molecule”, “donor template”, or “donor template molecule” (collectively a “donor template”), which may be a recombinant polynucleotide, DNA or RNA donor template or sequence, is defined as a nucleic acid molecule having a homologous nucleic acid template or sequence (e.g., homology sequence) and/or an insertion sequence for site-directed, targeted insertion or recombination into the genome of a plant cell via repair of a nick or DSB in the genome of a plant cell. A donor template may be a separate DNA molecule comprising one or more homologous sequence(s) and/or an insertion sequence for targeted integration, or a donor template may be a sequence portion (i.e., a donor template region) of a DNA molecule further comprising one or more other expression cassettes, genes/transgenes, and/or transcribable DNA sequences. For example, a “donor template” may be used for site-directed integration of a transgene or construct, or as a template to introduce a mutation, such as an insertion, deletion, substitution, etc., into a target site within the genome of a plant. A targeted genome editing technique provided herein may comprise the use of one or more, two or more, three or more, four or more, or five or more donor molecules or templates. A donor template provided herein may comprise at least one, at least two, at least three, at least four, at least five, at least six, at least seven, at least eight, at least nine, or at least ten gene(s) or transgene(s) and/or transcribable DNA sequence(s). Alternatively, a donor template may comprise no genes, transgenes or transcribable DNA sequences.

Without being limiting, a gene/transgene or transcribable DNA sequence of a donor template may include, for example, an insecticidal resistance gene, an herbicide tolerance gene, a nitrogen use efficiency gene, a water use efficiency gene, a yield enhancing gene, a nutritional quality gene, a DNA binding gene, a selectable marker gene, an RNAi or suppression construct, a site-specific genome modification enzyme gene, a single guide RNA of a CRISPR/Cas9 system, a geminivirus-based expression cassette, or a plant viral expression vector system. According to other embodiments, an insertion sequence of a donor template may comprise a protein encoding sequence or a transcribable DNA sequence that encodes a non-coding RNA molecule, which may target an endogenous gene for suppression. A donor template may comprise a promoter operably linked to a coding sequence, gene, or transcribable DNA sequence, such as a constitutive promoter, a tissue-specific or tissue-preferred promoter, a developmental stage promoter, or an inducible promoter. A donor template may comprise a leader, enhancer, promoter, transcriptional start site, 5′-UTR, one or more exon(s), one or more intron(s), transcriptional termination site, region or sequence, 3′-UTR, and/or polyadenylation signal, which may each be operably linked to a coding sequence, gene (or transgene) or transcribable DNA sequence encoding a non-coding RNA, a guide RNA, an mRNA and/or protein. A donor template may be a single-stranded or double-stranded DNA or RNA molecule or plasmid.

An “insertion sequence” of a donor template is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a plant.

Any method known in the art for site-directed integration may be used with the present disclosure. In the presence of a donor template molecule with an insertion sequence, the DSB or nick can be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, transcribable DNA sequence, construct, or sequence may be achieved if the transgene, transcribable DNA sequence, construct or sequence is located in the insertion sequence of the donor template.

The introduction of a DSB or nick may also be used to introduce targeted mutations in the genome of a plant, including genomic modifications that reduce or disrupt the activity of ZmDA1, as compared to the activity of ZmDA1 in an otherwise identical corn plant, corn plant seed, corn plant part, or corn plant cell that lacks the modification. As used herein, a “mutation” refers to the permanent alteration of the nucleotide sequence of the genome of an organism, the extrachromosomal DNA, or other genetic elements, e.g. targeted mutations within a genomic region from nucleotide position 3434 to nucleotide position 6811 with reference to sequence SEQ ID NO:3. According to this approach, mutations, such as deletions, insertions, substitutions, inversions, and/or duplications may be introduced at a target site via imperfect repair of the DSB or nick to produce a genetic modification within a gene. Such mutations may be generated by imperfect repair of the targeted locus even without the use of a donor template molecule. A modification of a gene may be achieved by inducing a DSB or nick at or near the endogenous locus of the gene that results in expression of a non-functional protein, interfering protein, or a protein having reduced, disrupted, or altered activity as compared to a protein expressed from the gene lacking said modification.

As used herein, the term “insertion” as it relates to a mutation, refers to the addition of one or more extra nucleotides into the DNA. Insertions in the coding region of a gene may alter splicing of the mRNA (splice site mutation) or cause a shift in the reading frame (frameshift), both of which can significantly alter the gene product.

As used herein, the term “deletion” as it relates to a mutation refers to the removal of one or more nucleotides from the DNA. Like insertion mutations, these mutations can alter the reading frame of the gene.

As used herein, the term “substitution” as it relates to a mutation refers to an exchange of a single nucleotide for another.

As used herein, the term “inversion” refers to reversing the orientation of a chromosomal segment. An inversion can be accompanied by a loss of nucleotides flanking either one or both sites of the inversion due to DNA repair mechanisms occurring at the cut and ligation sites during the formation of an inversion.

As used herein, the term “duplication” refers to the creation of multiple copies of chromosomal regions, increasing the dosage of the genes located within them.

As used herein, a “missense mutation” refers to a single nucleotide change that results in a codon that codes for a different amino acid. For example, the codon “CGU” encodes an arginine amino acid. If a missense mutation changes the G to a U, producing a “CUU” codon, the codon now encodes a leucine amino acid. Missense mutations can be caused by an insertion, deletion, substitution, duplication, or inversion. The frameshift, missense, or nonsense mutations described herein lead to loss of function or expression of a targeted gene, such as a ZmDA1 gene. A “loss-of-function mutation” is a mutation in the coding sequence of a gene, which causes the function of the gene product, usually a protein, to be either reduced or completely absent, e.g. reduction of peptidase activity. A loss-of-function mutation can, for instance, be caused by the truncation of the gene product. A phenotype associated with an allele with a loss of function mutation can be either recessive or dominant.

Similarly, such targeted mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, substitutions, inversions, and/or duplications, relative to the targeted genomic sequence at or near the site of the DSB or nick. For example, targeted mutations of a gene may be achieved by deleting, inserting, substituting, inverting, or duplicating at least a portion of the gene, such as by introducing a frame shift or premature stop codon into the coding sequence of the gene or introducing a modification into a transcribable DNA sequence. A deletion of a portion of a gene may also be introduced by generating DSBs or nicks at two target sites and causing a deletion of the intervening target region flanked by the target sites. A modification of a targeted gene may result in expression of a non-functional protein, interfering protein, or a protein having reduced, disrupted, or altered activity as compared to a protein expressed from the gene lacking said modification.

In an aspect, the present disclosure provides a modified corn plant, or plant seed, plant part or plant cell thereof, comprising a mutant allele of the ZmDA1 gene, wherein the mutant allele comprises at least one genome modification involving at least 1, at least 2, at least 3, at least 4, at least 6, at least 8, at least 10, at least 20, at least 30, at least 40, at least 50, at least 60, at least 70, at least 75, at least 80, at least 85, at least 90, at least 95, at least 100, at least 110, at least 115, at least 125, at least 150, at least 200, at least 300, at least 600, or at least 650 consecutive nucleotides of a transcribable region of the endogenous ZmDA1 gene. A transcribable DNA sequence of the ZmDA1 gene comprises the sequence of SEQ ID NO:3, which is an approximately 7 kb polynucleotide sequence within the ZmDA1 gene. The genome modification may be a deletion of a region comprising at least 1, at least 2, at least, 3, at least 4, at least 6, at least 8, at least 10, at least 20, at least 30, at least 40, at least 50, at least 60, at least 70, at least 75, at least 80, at least 85, at least 90, at least 95, at least 100, at least 110, at least 115, at least 125, least 150, at least 200, at least 300, at least 600 or at least 650 consecutive nucleotides within the sequence of SEQ ID NO:3. Such a deletion in SEQ ID NO:3 may include a region that spans: from nucleotide 4322 to nucleotide 4436; from nucleotide 4324 to nucleotide 4333; from nucleotide 4430 to nucleotide 4433; from nucleotide 4327 to nucleotide 4333; from nucleotide 4429 to nucleotide 4434; from nucleotide 3650 to nucleotide 3657; from nucleotide 4327 to nucleotide 4337; from nucleotide 4324 to nucleotide 4333; from nucleotide 4426 to nucleotide 4431; from nucleotide 4325 to nucleotide 4437; from nucleotide 4325 to nucleotide 4330; from nucleotide 4428 to nucleotide 4433; from nucleotide 4326 to nucleotide 4354; from nucleotide 4429 to nucleotide 4431; or from nucleotide 4325 to nucleotide 4435 of SEQ ID NO:3. In an aspect, the genome modification may also comprise a deletion and nucleotide substitutions or nucleotide insertions of at least 1, at least 2, at least 3, at least 4, at least 6, at least 8, at least 10, or at least 20 consecutive nucleotides around the deletion. Such a modification including nucleotide substitutions or nucleotide insertions in SEQ ID NO:3 may include a region of SEQ ID NO:3 that spans: from nucleotide 3650 to nucleotide 3657, wherein nucleotide 3657 is a nucleotide substitution or nucleotide insertion; from nucleotide 4325 to nucleotide 4435, wherein nucleotide 4325 is a nucleotide substitution or nucleotide insertion.

In an aspect, a mutant allele of the ZmDA1 gene may comprise two or more modifications in the transcribable region of the endogenous ZmDA1 gene. Examples of such mutant alleles of the ZmDA1 gene are disclosed herein and include, for example, an allele comprising two deletions in the sequence of SEQ ID NO:3, wherein the first deletion spans a region from nucleotide 4324 to nucleotide 4333 of SEQ ID NO:3 and the second deletion spans from nucleotide 4430 to nucleotide 4433 of SEQ ID NO:3; an allele comprising two deletions within the sequence of SEQ ID NO:3, where the first deletion spans a region from nucleotide 4327 to nucleotide 4333 of SEQ ID NO:3 and the second deletion spans from nucleotide 4429 to nucleotide 4434 of SEQ ID NO:3; an allele comprising two deletions within the sequence of SEQ ID NO:3, where the first deletion spans a region from nucleotide 3650 to nucleotide 3657 of SEQ ID NO:3 and the second deletion spans from nucleotide 4327 to nucleotide 4337 of SEQ ID NO:3; an allele comprising two deletions within the sequence of SEQ ID NO:3, where the first deletion spans a region from nucleotide 4324 to nucleotide 4333 of SEQ ID NO:3 and the second deletion spans from nucleotide 4426 to nucleotide 4431 of SEQ ID NO:3; an allele comprising two deletions within the sequence of SEQ ID NO:3, where the first deletion spans a region from nucleotide 4325 to nucleotide 4330 of SEQ ID NO:3 and the second deletion spans from nucleotide 4428 to nucleotide 4433 of SEQ ID NO:3; and an allele comprising two deletions within the sequence of SEQ ID NO:3, where the first deletion spans a region from nucleotide 4326 to nucleotide 4354 of SEQ ID NO:3 and the second deletion spans from nucleotide 4429 to nucleotide 4431 of SEQ ID NO:3; an allele comprising two modifications within the sequence of SEQ ID NO:3, where the first modification includes a deletion and a nucleotide substitution or nucleotide insertion and spans a region from nucleotide 3650 to nucleotide 3657 of SEQ ID NO:3, wherein nucleotide 3657 is a nucleotide substitution or nucleotide insertion and the second modification is a deletion that spans from nucleotide 4327 to nucleotide 4337 of SEQ ID NO:3.

In an aspect, a mutant allele of the ZmDA1 gene may comprise, for example, an allele comprising two deletions within the sequence of SEQ ID NO:3, where the first deletion spans a region from nucleotide 4328 to nucleotide 4337 of SEQ ID NO:3 and the second deletion spans from nucleotide 4429 to nucleotide 4431 of SEQ ID NO:3; an allele comprising two deletions within the sequence of SEQ ID NO:3, where the first deletion spans a region from nucleotide 4326 to nucleotide 4335 of SEQ ID NO:3 and the second deletion spans from nucleotide 4426 to nucleotide 4434 of SEQ ID NO:3; an allele comprising a deletion within the sequence of SEQ ID NO:3, wherein the deletion spans a region from nucleotide 4327 to nucleotide 4434 of SEQ ID NO:3; an allele comprising two deletions within the sequence of SEQ ID NO:3, where the first deletion spans a region from nucleotide 4324 to nucleotide 4335 of SEQ ID NO:3 and the second deletion spans from nucleotide 4427 to nucleotide 4437 of SEQ ID NO:3; an allele comprising two deletions within the sequence of SEQ ID NO:3, where the first deletion spans a region from nucleotide 4319 to nucleotide 4336 of SEQ ID NO:3 and the second deletion spans from nucleotide 4428 to nucleotide 4434 of SEQ ID NO:3; an allele comprising two deletions within the sequence of SEQ ID NO:3, where the first deletion spans a region from nucleotide 4334 to nucleotide 4339 of SEQ ID NO:3 and the second deletion spans from nucleotide 4429 to nucleotide 4432 of SEQ ID NO:3; an allele comprising two deletions within the sequence of SEQ ID NO:3, where the first deletion spans a region from nucleotide 3719 to nucleotide 4342 of SEQ ID NO:3 and the second deletion spans from nucleotide 4414 to nucleotide 4437 of SEQ ID NO:3; an allele comprising a deletion within the sequence of SEQ ID NO:3, wherein the deletion spans a region from nucleotide 4328 to nucleotide 4335 of SEQ ID NO:3; an allele comprising two deletions within the sequence of SEQ ID NO:3, where the first deletion spans a region from nucleotide 4326 to nucleotide 4334 of SEQ ID NO:3 and the second deletion spans from nucleotide 4427 to nucleotide 4433 of SEQ ID NO:3; an allele comprising a deletion within the sequence of SEQ ID NO:3, wherein nucleotide 4429 of SEQ ID NO:3 is deleted; an allele comprising a deletion within the sequence of SEQ ID NO:3, wherein the deletion spans a region from nucleotide 4426 to nucleotide 4435 of SEQ ID NO:3; an allele comprising a deletion within the sequence of SEQ ID NO:3, wherein the deletion spans a region from nucleotide 4431 to nucleotide 4434 of SEQ ID NO:3; an allele comprising a deletion within the sequence of SEQ ID NO:3, wherein the deletion spans a region from nucleotide 4427 to nucleotide 4433 of SEQ ID NO:3; an allele comprising a modification within the sequence of SEQ ID NO:3, where the modification includes a deletion and a nucleotide substitution or nucleotide insertion spanning a region from nucleotide 4428 to nucleotide 4434 of SEQ ID NO:3; an allele comprising a deletion within the sequence of SEQ ID NO:3, wherein the deletion spans a region from nucleotide 4326 to nucleotide 4433 of SEQ ID NO:3; an allele comprising a deletion within the sequence of SEQ ID NO:3, wherein the deletion spans a region from nucleotide 4331 to nucleotide 4442 of SEQ ID NO:3; an allele comprising two deletions within the sequence of SEQ ID NO:3, where the first deletion spans a region from nucleotide 4331 to nucleotide 4333 of SEQ ID NO:3 and the second deletion spans from nucleotide 4429 to nucleotide 4432 of SEQ ID NO:3; an allele comprising a deletion within the sequence of SEQ ID NO:3, wherein the deletion spans a region from nucleotide 4421 to nucleotide 4436 of SEQ ID NO:3; an allele comprising a modification within the sequence of SEQ ID NO:3, where the modification includes a deletion and a nucleotide substitution or nucleotide insertion spanning a region from nucleotide 4428 to nucleotide 4435 of SEQ ID NO:3; an allele comprising a deletion within the sequence of SEQ ID NO:3, wherein the deletion spans a region from nucleotide 4429 to nucleotide 4433 of SEQ ID NO:3; an allele comprising a deletion within the sequence of SEQ ID NO:3, wherein the deletion spans a region from nucleotide 4325 to nucleotide 4334 of SEQ ID NO:3; an allele comprising a deletion within the sequence of SEQ ID NO:3, wherein the deletion spans a region from nucleotide 4107 to nucleotide 4340 of SEQ ID NO:3; an allele comprising a modification within the sequence of SEQ ID NO:3, where the modification includes a deletion and a nucleotide substitution or nucleotide insertion spanning a region from nucleotide 4327 to nucleotide 4335 of SEQ ID NO:3; an allele comprising a deletion within the sequence of SEQ ID NO:3, wherein the deletion spans a region from nucleotide 4431 to nucleotide 4433 of SEQ ID NO:3; an allele comprising a deletion within the sequence of SEQ ID NO:3, wherein the deletion spans a region from nucleotide 4428 to nucleotide 4437 of SEQ ID NO:3; an allele comprising a deletion within the sequence of SEQ ID NO:3, wherein the deletion spans a region from nucleotide 4326 to nucleotide 4350 of SEQ ID NO:3; an allele comprising a deletion within the sequence of SEQ ID NO:3, wherein the deletion spans a region from nucleotide 4328 to nucleotide 4335 of SEQ ID NO:3; an allele comprising a modification within the sequence of SEQ ID NO:3, where the modification includes a deletion and a nucleotide substitution or nucleotide insertion spanning a region from nucleotide 4425 to nucleotide 4433 of SEQ ID NO:3; an allele comprising a deletion within the sequence of SEQ ID NO:3, wherein the deletion spans a region from nucleotide 4429 to nucleotide 4431 of SEQ ID NO:3; an allele comprising two deletions within the sequence of SEQ ID NO:3, where the first deletion spans a region from nucleotide 4325 to nucleotide 4334 of SEQ ID NO:3 and the second deletion spans from nucleotide 4429 to nucleotide 4433 of SEQ ID NO:3; an allele comprising two deletions within the sequence of SEQ ID NO:3, where the first deletion spans a region from nucleotide 4107 to nucleotide 4340 of SEQ ID NO:3 and the second deletion spans from nucleotide 4428 to nucleotide 4437 of SEQ ID NO:3; an allele comprising two modifications within the sequence of SEQ ID NO:3, where the first modification includes a deletion and a nucleotide substitution or nucleotide insertion spanning a region from nucleotide 4327 to nucleotide 4335 of SEQ ID NO:3 and the second modification includes a deletion spanning a region from nucleotide 4431 to nucleotide 4433 of SEQ ID NO:3; an allele comprising two deletions within the sequence of SEQ ID NO:3, where the first deletion spans a region from nucleotide 4107 to nucleotide 4340 of SEQ ID NO:3 and the second deletion spans from nucleotide 4431 to nucleotide 4433 of SEQ ID NO:3; an allele comprising two modifications within the sequence of SEQ ID NO:3, where the first modification includes a deletion and a nucleotide substitution or nucleotide insertion spanning a region from nucleotide 4327 to nucleotide 4335 of SEQ ID NO:3 and the second modification includes a deletion spanning a region from nucleotide 4428 to nucleotide 4437 of SEQ ID NO:3; an allele comprising two deletions within the sequence of SEQ ID NO:3, where the first deletion spans a region from nucleotide 4326 to nucleotide 4350 of SEQ ID NO:3 and the second deletion spans from nucleotide 4429 to nucleotide 4431 of SEQ ID NO:3; an allele comprising two modifications within the sequence of SEQ ID NO:3, where the first modification includes a deletion spanning a region from nucleotide 4328 to nucleotide 4335 of SEQ ID NO:3 and the second modification includes a deletion and a nucleotide substitution or nucleotide insertion spanning a region from nucleotide 4425 to nucleotide 4433 of SEQ ID NO:3; an allele comprising two modifications within the sequence of SEQ ID NO:3, where the first modification includes a deletion spanning a region from nucleotide 4326 to nucleotide 4350 of SEQ ID NO:3 and the second modification includes a deletion and a nucleotide substitution or nucleotide insertion spanning a region from nucleotide 4425 to nucleotide 4433 of SEQ ID NO:3; an allele comprising two deletions within the sequence of SEQ ID NO:3, where the first deletion spans a region from nucleotide 4328 to nucleotide 4335 of SEQ ID NO:3 and the second deletion spans from nucleotide 4429 to nucleotide 4431 of SEQ ID NO:3.

Other targeted modifications may be made in the transcribable region to generate novel alleles in the ZmDA1 gene. For example, one or more modification sites may be located at about 2375 nucleotides or more upstream from the 3′ end of reference sequence SEQ ID NO:3; or one or more modification sites may be located at about 2369 nucleotides or more upstream from the 3′ end of reference sequence SEQ ID NO:3. One or more modification sites may be also be located at about 3650 nucleotides or more downstream from the 5′ end of reference sequence SEQ ID NO:3. In an aspect, one or more modifications may be made within the region of DNA spanning from nucleotide position 3434 to nucleotide position 6811 of SEQ ID NO:3 to generate a novel allele in the ZmDA1 gene. In another aspect, one or more modifications may be made within the region of DNA spanning from about nucleotide position 3650 to about nucleotide position 4437 of SEQ ID NO:3 to generate a novel allele in the ZmDA1 gene. In another aspect, the modification may be made within a genomic region from about nucleotide position 3650 to about nucleotide position 4443 of reference sequence SEQ ID NO:3 to generate a novel allele in the ZmDA1 gene. In yet another aspect, the modification may be made within a genomic region from about nucleotide position 3719 to about nucleotide position 4443 of reference sequence SEQ ID NO:3 to generate a novel allele in the ZmDA1 gene.

In another aspect, a modified corn plant, corn plant seed, corn plant part, or corn plant cell provided herein may, in certain embodiments, comprise at least one modification, wherein the modification comprises a deletion, an insertion, a substitution, an inversion, a duplication, or any combination thereof, in at least one allele of an endogenous ZmDA1 gene. In a further aspect, the modification is defined by at least one of SEQ ID NOs:11-49. For example, a modified plant, plant seed, plant part, or plant cell provided herein may comprise a modification in at least one allele of the ZmDA1 gene, wherein the modification comprises a 115 base pair deletion defined by SEQ ID NO:11.

In a further aspect, the present disclosure provides a modified corn plant, or plant seed, plant part, or plant cell thereof, comprising a mutant allele of the ZmDA1 gene, wherein the mutant allele comprises one or more junction sequences, wherein the junction sequences are at least 30, at least 60, at least 100 nucleotides at the junction site. As used herein a “junction site” is the connection point between the nucleotide sequences at the site of a deletion, insertion, substitution, inversion, duplication, or any combination thereof. In the case of a deletion, the junction site is the connection point at the site of the deletion of the sequences that previously flanked the deletion. For example, in the case of the 115 base pair deletion from nucleotide 4322 to nucleotide 4436, as compared to reference sequence SEQ ID NO:3 described herein, the junction site would be between nucleotide 4321 and nucleotide 4437. In the case of a modification comprising a deletion, and a nucleotide substitution or nucleotide insertion around the deletion, the junction site is the connection point at the site of the modification that previously flanked the deletion. For example, in the case of the 8 base pair modification from nucleotide 3650 to nucleotide 3657, wherein nucleotide 3657 is a nucleotide substitution or nucleotide insertion as compared to reference sequence SEQ ID NO:3 described herein, the junction site would be between nucleotide 3649 and nucleotide 3657. In the case of an insertion, substitution, inversion, or duplication the junction site is the connection point between the inserted, substituted, inverted, or duplicated sequence and the flanking DNA sequences. In the case of an insertion, substitution, inversion, or duplication, one junction site is found at the 5′ end of the insertion, substitution, inversion, or duplication, and another junction site is found at the 3′ end of the insertion, substitution, inversion, or duplication. A “junction sequence” refers to a DNA sequence of any length that spans a junction site. A junction sequence can comprise at least 3 nucleotides, at least 10 nucleotides, at least, 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, or more. As one illustrative example, in the case of the 115 base pair deletion from nucleotide 4322 to nucleotide 4436, as compared to reference sequence SEQ ID NO:3 described herein, a junction sequence may be defined, in certain embodiments, as comprising a nucleotide sequence from nucleotide 4317 to nucleotide 4326 of SEQ ID NO:11, from nucleotide 4312 to nucleotide 4331 of SEQ ID NO:11, from nucleotide 4307 to nucleotide 4336 of SEQ ID NO:11, from nucleotide 4302 to nucleotide 4341 of SEQ ID NO:11, from nucleotide 4297 to nucleotide 4346 of SEQ ID NO:11, from nucleotide 4292 to nucleotide 4351 of SEQ ID NO:11, from nucleotide 4287 to nucleotide 4356 of SEQ ID NO:11, from nucleotide 4282 to nucleotide 4361 of SEQ ID NO:11, from nucleotide 4277 to nucleotide 4366 of SEQ ID NO:11, from nucleotide 4272 to nucleotide 4371 of SEQ ID NO:11, from nucleotide 4222 to nucleotide 4421 of SEQ ID NO:11, or from nucleotide 4172 to nucleotide 4471 of SEQ ID NO:11.

### II. Constructs for Genome Editing

Recombinant DNA constructs and vectors are provided comprising a polynucleotide sequence encoding a site-specific nuclease, such as a zinc-finger nuclease (ZFN), a meganuclease, an RNA-guided endonuclease, a TALE-endonuclease (TALEN), a recombinase, or a transposase, wherein the coding sequence is operably linked to a plant expressible promoter. For RNA-guided endonucleases, recombinant DNA constructs and vectors are further provided comprising a polynucleotide sequence encoding a guide RNA, wherein the guide RNA comprises a guide sequence of sufficient length having a percent identity or complementarity to a target site within the genome of a plant, such as at or near a targeted ZmDA1 gene. A polynucleotide sequence of a recombinant DNA construct and vector that encodes a site-specific nuclease or a guide RNA may be operably linked to a plant expressible promoter, such as an inducible promoter, a constitutive promoter, a tissue-specific promoter, etc.

In an aspect, vectors comprising polynucleotides encoding a site-specific nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In an aspect, vectors comprising polynucleotides encoding a Cpf1 nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In another aspect, vectors comprising polynucleotides encoding a Cpf1 and, optionally one or more, two or more, three or more, or four or more crRNAs are provided to a cell by transformation methods known in the art (e.g., without being limiting, viral transfection, particle bombardment, PEG-mediated protoplast transfection or Agrobacterium-mediated transformation).

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, “locus” is a chromosomal locus or region where a polymorphic nucleic acid, trait determinant, gene, or marker is located. A “locus” can be shared by two homologous chromosomes to refer to their corresponding locus or region. As used herein, an “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant or edited allele for a gene may have reduced, disrupted, altered, or eliminated activity, or a reduced or eliminated expression level for the gene relative to the wild-type allele. For example, a mutant or edited allele for the ZmDA1 gene may have a deletion in the transcribable region of the endogenous ZmDA1 gene that reduces, disrupts, or alters the activity of the protein encoded by the mutant allele as compared to the activity of the protein encoded by the wild-type allele in an otherwise identical corn plant. For diploid organisms such as corn, a first allele can occur on one chromosome, and a second allele can occur at the same locus on a second homologous chromosome. If one allele at a locus on one chromosome of a plant is a mutant or edited allele and the other corresponding allele on the homologous chromosome of the plant is wild-type, then the plant is described as being heterozygous for the mutant or edited allele. However, if both alleles at a locus are mutant or edited alleles, then the plant is described as being homozygous or biallelic for the mutant or edited alleles. As used herein, the term “homozygous” refers to a genotype comprising two identical alleles at a given locus in a diploid genome. Given that corn is a diploid organism, CRISPR-mediated gene editing can result in biallelic (that is, different edits are made to the same locus on corresponding homologous chromosomes) edits resulting in a genotype comprising two non-identical mutant alleles at a given locus in a diploid genome in R₀ plants. When used in the context of edited alleles, plants comprising such genotypes may also be referred to as comprising a heteroallelic combination or biallelic edits.

As used herein, a “wild-type gene” or “wild-type allele” refers to a gene or allele having a sequence or genotype that is most common in a particular plant species, or another sequence or genotype having only natural variations, polymorphisms, or other silent mutations relative to the most common sequence or genotype that do not significantly impact the expression and activity of the gene or allele. Indeed, a “wild-type” gene or allele contains no variation, polymorphism, or any other type of mutation that substantially affects the normal function, activity, expression, or phenotypic consequence of the gene or allele relative to the most common sequence or genotype.

In general, the term “variant” refers to molecules with some differences, generated synthetically or naturally, in their nucleotide or amino acid sequences as compared to a reference (native) polynucleotides or polypeptides, respectively. These differences include substitutions, insertions, deletions, inversions, duplications, or any desired combinations of such changes in a native polynucleotide or amino acid sequence.

As used herein, the term “expression” refers to the biosynthesis of a gene product, and typically the transcription and/or translation of a nucleotide sequence, such as an endogenous gene, a heterologous gene, a transgene or an RNA and/or protein coding sequence, in a cell, tissue, organ, or organism, such as a plant, plant part or plant cell, tissue or organ.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., can comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other.

Such a recombinant polynucleotide molecule, protein, construct, etc., can also refer to a polynucleotide or protein molecule or sequence that has been genetically engineered and/or constructed outside of a cell. For example, a recombinant DNA molecule can comprise any engineered or man-made plasmid, vector, etc., and can include a linear or circular DNA molecule. Such plasmids, vectors, etc., can contain various maintenance elements including a prokaryotic origin of replication and selectable marker, as well as one or more transgenes or expression cassettes perhaps in addition to a plant selectable marker gene, etc. The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s).

Reference in this application to an “isolated DNA molecule” or an “isolated polynucleotide”, or an equivalent term or phrase, is intended to mean that the DNA molecule or polynucleotide is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding a protein or any naturally occurring variant of that protein would be an isolated nucleotide sequence so long as the nucleotide sequence was not within the DNA of the organism in which the sequence encoding the protein is naturally found. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

As commonly understood in the art, the term “promoter” can generally refer to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied or derived from a known or naturally occurring promoter sequence or other promoter sequence. A promoter can also include a chimeric promoter comprising a combination of two or more heterologous sequences. A promoter of the present disclosure can thus include variants or fragments of promoter sequences that are similar in composition, but not identical to, other promoter sequence(s) known or provided herein. A promoter provided herein, or variant or fragment thereof, may comprise a “minimal promoter” which provides a basal level of transcription and is comprised of a TATA box or equivalent DNA sequence for recognition and binding of the RNA polymerase II complex for initiation of transcription. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of the plant are referred to as “constitutive” promoters. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. An “inducible” promoter is a promoter that initiates transcription in response to an environmental stimulus such as cold, drought or light, or other stimuli, such as wounding or chemical application. A promoter can also be classified in terms of its origin, such as being heterologous, homologous, chimeric, synthetic, etc.

As used herein, a “plant-expressible promoter” refers to a promoter that can initiate, assist, affect, cause, and/or promote the transcription and expression of its associated transcribable DNA sequence, coding sequence or gene in a plant cell or tissue.

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature without human introduction - e.g., the promoter or regulatory sequence has a different origin relative to the associated polynucleotide sequence and/or the promoter or regulatory sequence is not naturally occurring in a plant species to be transformed with the promoter or regulatory sequence.

As used herein, an “endogenous gene” or an “endogenous locus” refers to a gene or locus at its natural and original chromosomal location. As used herein, the “endogenous ZmDA1 gene” refers to the ZmDA1 genomic locus at its original chromosomal location.

As used herein, in the context of a protein-coding gene, an “exon” refers to a segment of a DNA or RNA molecule containing information coding for a protein or polypeptide sequence.

As used herein, an “intron” of a gene refers to a segment of a DNA or RNA molecule, which does not contain information coding for a protein or polypeptide, and which is first transcribed into an RNA sequence but then spliced out from a mature RNA molecule.

As used herein, an “untranslated region (UTR)” of a gene refers to a segment of an RNA molecule or sequence (e.g., a mRNA molecule) expressed from a gene (or transgene), but excluding the exon and intron sequences of the RNA molecule. An “untranslated region (UTR)” also refers to a DNA segment or sequence encoding such a UTR segment of an RNA molecule. An untranslated region can be a 5′-UTR or a 3′-UTR depending on whether it is located at the 5′ or 3′ end of a DNA or RNA molecule or sequence relative to a coding region of the DNA or RNA molecule or sequence (i.e., upstream (5′) or downstream (3′) of the exon and intron sequences, respectively).

As used herein, a “transcribable region” or “transcribable DNA sequence” refers to a nucleic acid sequence expressed from a gene (or transgene).

As used herein, a “transcription termination sequence” refers to a nucleic acid sequence containing a signal that triggers the release of a newly synthesized transcript RNA molecule from an RNA polymerase complex and marks the end of transcription of a gene or locus.

The terms “percent identity,” “% identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.” Sequences having a percent identity to a base sequence may exhibit the activity of the base sequence.

Degeneracy of the genetic code provides the possibility to substitute at least one base of the protein encoding sequence of a gene with a different base without causing the amino acid sequence of the polypeptide produced from the gene to be changed. When optimally aligned, homolog proteins, or their corresponding nucleotide sequences, have typically at least about 70%, at least about 75%, at least about 80%, at least about 85%, at least about 90%, at least about 92%, at least about 93%, at least about 94%, at least about 95%, at least about 96%, at least about 97%, at least about 98%, at least about 99%, or even at least about 99.5% identity over the full length of a protein or its corresponding nucleotide sequence identified as being associated with imparting an altered phenotype when expressed in plant cells. According to embodiments of the present invention, a ZmDA1 gene encodes a protein having at least 70%, at least 75%, at least 80%, at least 85%, at least 90%, at least 95%, at least 96%, at least 97%, at least 98%, at least 99%, at least 99.5%, or 100% sequence identity to SEQ ID NO:2.

Homologs are inferred from sequence similarity, by comparison of protein sequences, for example, manually or by use of a computer-based tool. For optimal alignment of sequences to calculate their percent identity, various pair-wise or multiple sequence alignment algorithms and programs are known in the art, such as ClustalW or Basic Local Alignment Search Tool® (BLAST), etc., that can be used to compare the sequence identity or similarity between two or more nucleotide or protein sequences. BLAST, can also be used, for example to search query protein sequences of a base organism against a database of protein sequences of various organisms, to find similar sequences. The generated summary Expectation value (E-value) can be used to measure the level of sequence similarity. Because a protein hit with the lowest E-value for a particular organism may not necessarily be an ortholog or be the only ortholog, a reciprocal query is used to filter hit sequences with significant E-values for ortholog identification. The reciprocal query entails search of the significant hits against a database of protein sequences of the base organism. A hit can be identified as an ortholog, when the reciprocal query’s best hit is the query protein itself or a paralog of the query protein. With the reciprocal query process orthologs are further differentiated from paralogs among all the homologs, which allows for the inference of functional equivalence of genes.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and an RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

As used herein, a “fragment” of a polynucleotide refers to a sequence comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule or protein as disclosed herein. Methods for producing such fragments from a starting promoter molecule are well known in the art. Fragments of a DNA molecule or protein may exhibit the activity of the DNA molecule or protein from which they are derived.

A plant selectable marker transgene in a transformation vector or construct of the present disclosure may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptll), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (proA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. Plant transformation may also be carried out in the absence of selection during one or more steps or stages of culturing, developing or regenerating transformed explants, tissues, plants and/or plant parts.

### III. Transformation Methods

Methods and compositions are provided for transforming a plant cell, tissue or explant with a recombinant DNA molecule or construct encoding one or more molecules required for targeted genome editing (e.g., guide RNA(s) and/or site-directed nuclease(s)). Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are bacterially-mediated transformation, such as Agrobacterium-mediated or Rhizobium-mediated transformation, and microprojectile or particle bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616. Other methods for plant transformation, such as microinjection, electroporation, vacuum infiltration, pressure, sonication, silicon carbide fiber agitation, PEG-mediated transformation, etc., are also known in the art.

Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants, also referred to as R₀ plants. As used herein, “R₀ plant” refers to an initial regenerated transformant. As used herein, “R₁ seed” refers to seed produced from selfing R₀ plants. As used herein, “R₁ plant” refers to a plant grown from R₁ seed. As used herein, “R₂ seed” refers to seed produced from selfing R₁ plants. As used herein, “R₂ plant” refers to a plant grown from R₂ seed. As provided herein, following one to two generations of self-crossing of R₀ plants, plants homozygous for edited alleles of the ZmDA1 edited region, devoid of editing T-DNA sequences, may be produced. Furthermore, such modified plants may be crossed with a different WT male corn plant line to produce hybrid plants.

Any suitable method or technique for transformation of a plant cell known in the art may be used according to present methods. In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes.

As used herein, the terms “regeneration” and “regenerating” refer to a process of growing or developing a plant from one or more plant cells through one or more culturing steps. Transformed or edited cells, tissues or explants containing a DNA sequence insertion or edit may be grown, developed or regenerated into transgenic plants in culture, plugs, or soil according to methods known in the art. Certain embodiments of the disclosure therefore relate to methods and constructs for regenerating a plant from a cell with modified genomic DNA resulting from genome editing. The regenerated plant can then be used to propagate additional plants.

According to an aspect of the present disclosure, regenerated plants or a progeny plant, plant part or seed thereof can be screened or selected based on a marker, trait, or phenotype produced by the edit or mutation, or by the site-directed integration of an insertion sequence, transgene, etc., in the developed or regenerated plant, or a progeny plant, plant part or seed thereof. If a given mutation, edit, trait or phenotype is recessive, one or more generations or crosses (e.g., selfing) from the initial R₀ plant may be necessary to produce a plant homozygous for the edit or mutation so the trait or phenotype can be observed. Progeny plants, such as plants grown from R₁ seed or in subsequent generations, can be tested for zygosity using any known zygosity assay, such as by using a single nucleotide polymorphism (SNP) assay, DNA sequencing, thermal amplification, or polymerase chain reaction (PCR), and/or Southern blotting that allows for the distinction between heterozygote, homozygote and wild-type plants.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. As used herein, a “molecular technique” refers to any method known in the fields of molecular biology, biochemistry, genetics, plant biology, or biophysics that involves the use, manipulation, or analysis of a nucleic acid, a protein, or a lipid. Without being limiting, molecular techniques useful for detecting the presence of a modified sequence in a genome include phenotypic screening; molecular marker technologies such as SNP analysis by TagMan® or Illumina/Infinium technology; Southern blot; PCR (including amplicon sequencing which consists of the generation of one or more unique PCR products across the genomic region of interest for further sequencing analysis, e.g., using Next-Gen Sequencing techniques known in the art. Sequence data from each sample is then mapped to a reference sequence to identify consensus differences); enzyme-linked immunosorbent assay (ELISA); and sequencing (e.g., Sanger, Illumina®, 454, Pac-Bio, Ion Torrent™). In one aspect, a method of detection provided herein comprises phenotypic screening. In another aspect, a method of detection provided herein comprises SNP analysis. In a further aspect, a method of detection provided herein comprises a Southern blot. In a further aspect, a method of detection provided herein comprises PCR. In a further aspect, a method of detection provided herein comprises amplicon sequencing. In an aspect, a method of detection provided herein comprises ELISA. In a further aspect, a method of detection provided herein comprises determining the sequence of a nucleic acid or a protein. Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Nucleic acids can be isolated using techniques routine in the art. For example, nucleic acids can be isolated using any method including, without limitation, recombinant nucleic acid technology, and/or PCR. General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials. The screening and selection of modified (e.g., edited) plants or plant cells can be through any methodologies known to those skilled in the art of molecular biology. Examples of screening and selection methodologies include, but are not limited to, Southern analysis, PCR amplification for detection of a polynucleotide (including amplicon sequencing), Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

### IV. Genome Modified Plants

As used herein, “modified” in the context of a corn plant, corn plant seed, corn plant part, corn plant cell, and/or corn plant genome, refers to a corn plant, plant seed, plant part, plant cell, and/or plant genome comprising an engineered change in the expression level and/or endogenous sequence of one or more genes of interest relative to a wild-type or control corn plant, plant seed, plant part, plant cell, and/or plant genome. Indeed, the term “modified” may further refer to a corn plant, plant seed, plant part, plant cell, and/or plant genome having one or more deletions and/or one or more nucleotide substitutions or nucleotide insertions affecting an endogenous ZmDA1 gene introduced through chemical mutagenesis, transposon insertion or excision, or any other known mutagenesis technique, or introduced through genome editing. In an aspect, a modified plant, plant seed, plant part, plant cell, and/or plant genome can comprise one or more transgenes. For clarity, therefore, a modified corn plant, plant seed, plant part, plant cell, and/or plant genome includes a mutated, edited and/or transgenic corn plant, plant seed, plant part, plant cell, and/or plant genome having a modified sequence of a ZmDA1 gene relative to a wild-type or control plant, plant seed, plant part, plant cell, and/or plant genome. Furthermore, the modification may reduce, disrupt, or alter the activity of the protein encoded by the ZmDA1 gene as compared to the activity of the protein encoded by the ZmDA1 gene in an otherwise identical corn plant.

Modified corn plants, plant parts, seeds, etc., may have been subjected to mutagenesis, genome editing or site-directed integration, genetic transformation, or a combination thereof. Such “modified” corn plants, plant seeds, plant parts, and plant cells include plants, plant seeds, plant parts, and plant cells that are offspring or derived from “modified” corn plants, plant seeds, plant parts, and plant cells that retain the molecular change (e.g., change in expression level and/or activity) to the ZmDA1 gene. A modified seed provided herein may give rise to a modified plant provided herein. A modified plant, plant seed, plant part, plant cell, or plant genome provided herein may comprise a recombinant DNA construct or vector or genome edit as provided herein.

A “modified plant product” may be any product made from a modified plant, plant part, plant cell, or plant chromosome provided herein, or any portion or component thereof. For example, in some embodiments a modified plant product may be a commodity product produced from a modified plant or part thereof containing the recombinant DNA molecule as described herein, such as those provided as SEQ ID NOs:11-49. In some embodiments, commodity products contain a detectable amount of DNA comprising a DNA sequence selected from the group consisting of SEQ ID NOs:11-49 or fragments or variants thereof. As used herein, a “commodity product” refers to any composition or product which is comprised of material derived from a modified plant, seed, plant cell, or plant part containing the DNA molecule as described herein, such as those provided as SEQ ID NOs:11-49. Commodity products include but are not limited to processed seeds, grains, plant parts, and meal, protein concentrate, protein isolate, grain, starch, flour, biomass, or seed oil. A commodity product containing a detectable amount of DNA corresponding to the recombinant DNA molecule as described herein, such as those provided as SEQ ID NOs:11-49 is contemplated. Detection of one or more of this DNA in a sample may be used for determining the content or the source of the commodity product. Any standard method of detection for DNA molecules may be used, including methods of detection disclosed herein.

Modified plants may be further crossed to themselves or other plants to produce modified plant seeds and progeny. A modified plant may also be prepared by crossing a first plant comprising a DNA sequence or construct or an edit (e.g., a genomic deletion) with a second plant lacking the DNA sequence or construct or edit. For example, a DNA sequence or inversion may be introduced into a first plant line that is amenable to transformation or editing, which may then be crossed with a second plant line to introgress the DNA sequence or edit (e.g., deletion) into the second plant line. Progeny of these crosses can be further backcrossed into the desirable line multiple times, such as through 6 to 8 generations or back crosses, to produce a progeny plant with substantially the same genotype as the original parental line, but for the introduction of the DNA sequence or edit. A modified plant, plant cell, or seed provided herein may be a hybrid plant, plant cell, or seed. As used herein, a “hybrid” is created by crossing two plants from different varieties, lines, inbreds, or species, such that the progeny comprises genetic material from each parent. Skilled artisans recognize that higher order hybrids can be generated as well.

A modified corn plant, plant part, plant cell, or seed provided herein may be of an elite variety or an elite line. An “elite variety” or an “elite line” refers to a variety that has resulted from breeding and selection for superior agronomic performance.

As used herein, the term “control plant” (or likewise a “control” plant seed, plant part, plant cell, and/or plant genome) refers to a corn plant (or plant seed, plant part, plant cell, and/or plant genome) that is used for comparison to a modified plant (or modified plant seed, plant part, plant cell, and/or plant genome) and has the same or similar genetic background (e.g., same parental lines, hybrid cross, inbred line, testers, etc.) as the modified plant (or plant seed, plant part, plant cell, and/or plant genome), except for genome edit(s) (e.g., a deletion) affecting a ZmDA1 gene. For example, a control plant may be an inbred line that is the same as the inbred line used to make the modified corn plant, or a control plant may be the product of the same hybrid cross of inbred parental lines as the modified plant, except for the absence in the control plant of any transgenic events or genome edit(s) affecting a ZmDA1 gene. Similarly, an “unmodified control plant” refers to a plant that shares a substantially similar or essentially identical genetic background as a modified plant, but without the one or more engineered changes to the genome (e.g., mutation or edit) of the modified plant. For purposes of comparison to a modified plant, plant seed, plant part, plant cell, and/or plant genome, a “wild-type plant” (or likewise a “wild-type” plant seed, plant part, plant cell, and/or plant genome) refers to a non-transgenic and non-genome edited control plant, plant seed, plant part, plant cell, and/or plant genome. As used herein, a “control” plant, plant seed, plant part, plant cell, and/or plant genome may also be a plant, plant seed, plant part, plant cell, and/or plant genome having a similar (but not the same or identical) genetic background to a modified plant, plant seed, plant part, plant cell, and/or plant genome, if deemed sufficiently similar for comparison of the characteristics or traits to be analyzed.

As used herein, the term “activity” refers to the biological function of a gene or protein. A gene or a protein may provide one or more distinct functions. A reduction, disruption, or alteration in “activity” thus refers to a lowering, reduction, or elimination of one or more functions of a gene or a protein in a corn plant, plant cell, or plant tissue at one or more stage(s) of plant development, as compared to the activity of the gene or protein in a wild-type or control plant, cell, or tissue at the same stage(s) of plant development. Additionally, an increase in “activity” thus refers to an elevation of one or more functions of a gene or a protein in a corn plant, plant cell, or plant tissue at one or more stage(s) of plant development, as compared to the activity of the gene or protein in a wild-type or control plant, cell, or tissue at the same stage(s) of plant development.

According to some embodiments, a modified corn plant is provided having a genomic modification in a ZmDA1 gene that results in reduced, disrupted, or altered activity of the protein encoded by the ZmDA1 gene in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to further embodiments, a modified corn plant is provided having a protein encoded by a ZmDA1 gene that results in reduced, disrupted, or altered activity in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%- 75%, 30%-80%, or 10%-75%, as compared to a control plant.

According to some embodiments, a modified plant is provided having a ZmDA1 mRNA level that is reduced or increased in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having a ZmDA1 mRNA expression level that is reduced or increased in at least one plant tissue by 5%-20%, 5%-25%, 5%- 30%, 5%-40%, 5%-50%, 5%-60%, 5%-70%, 5%-75%, 5%-80%, 5%-90%, 5%-100%, 75%-100%, 50%-100%, 50%-90%, 0%-75%, 25%-75%, 30%-80%, or 0%-75%, as compared to a control plant. According to some embodiments, a modified plant is provided having a ZmDA1 protein expression level that is reduced or increased in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having a ZmDA1 protein expression level that is reduced or increased in at least one plant tissue by 5%-20%, 5%- 25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant.

The present disclosure relates to a plant with improved economically important characteristics, including but not limited to increased yield. More specifically with respect to yield, the present disclosure relates to a modified plant comprising a genomic edit or mutation as described herein, wherein the plant exhibits increases in key yield-related traits, e.g., an increase in number of kernels per ear, number of kernels per longitudinal row of ear (i.e., kernel rank), ear size related traits (e.g., ear area, ear length, and ear diameter), single kernel weight, kernel row number, yield, grain yield estimate per plant, and/or grain yield estimate as compared to a control plant. Many plants of this disclosure exhibited increased or improved yield trait components as compared to a control plant. Yield can be defined as the measurable produce of economic value from a crop. Yield can be defined in the scope of quantity and/or quality. For example, corn yield can include grain weight per area measurement, grain weight per plant, number of ears per acre, or any other like conversion of harvested grain per unit measurement. Yield can be directly dependent on several factors, for example, the number and size of organs, plant architecture (such as the number of branches, plant biomass, etc.), flowering time and duration, grain fill period. Root architecture and development, photosynthetic efficiency, nutrient uptake, stress tolerance, early vigor, delayed senescence and functional stay green phenotypes can be important factors in determining yield. Optimizing the above-mentioned factors can therefore contribute to increasing crop yield.

Modified plants comprising or derived from plant cells that comprise a genome modification of this disclosure can be further enhanced with stacked traits, for example, a modified crop plant having an enhanced trait resulting from expression of DNA disclosed herein in combination with one or more additional genome modifications that provide a beneficial agronomic trait or further improve the enhanced trait.

Modified plants comprising or derived from plant cells that are transformed with a recombinant DNA of this disclosure can be further enhanced with stacked traits, for example, a modified crop plant having an enhanced trait resulting from expression of DNA disclosed herein in combination with one or more genes of agronomic interest that provide a beneficial agronomic trait (such as herbicide and/or pest resistance traits) to crop plants. For example, the traits conferred by the recombinant DNA constructs of the current disclosure can be stacked with other traits of agronomic interest, such as a trait providing insect resistance such as using a gene from Bacillus thuringensis to provide resistance against lepidopteran, coleopteran, homopteran, hemiopteran, and other insects, or improved quality traits such as improved nutritional value. Molecules and methods for imparting insect/nematode/virus resistance are disclosed in U.S. Pat. Nos. 5,250,515; 5,880,275; 6,506,599; 5,986,175; and U.S. Pat. Application Publication No. 2003/0150017 A1.

Herbicides for which transgenic plant tolerance has been demonstrated and the methods and compositions of the present disclosure can be applied include, but are not limited to, glyphosate, dicamba, glufosinate, sulfonylurea, bromoxynil, norflurazon, 2,4-D (2,4-dichlorophenoxy) acetic acid, aryloxyphenoxy propionates, p-hydroxyphenyl pyruvate dioxygenase inhibitors (HPPD), and protoporphyrinogen oxidase inhibitors (PPO) herbicides. Polynucleotide molecules encoding proteins involved in herbicide tolerance known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Pat. No. Application Publication No. 2003/0083480A1 also for imparting glyphosate tolerance; dicamba monooxygenase disclosed in U.S. Pat. Application Publication No. 2003/0135879A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al. (Plant J. 4:833-840, 1993) and in Misawa et al. (Plant J. 6:481-489, 1994) for norflurazon tolerance; a polynucleotide molecule encoding acetohydroxyacid synthase (AHAS, aka ALS) described in Sathasivan et al. (Nucl. Acids Res. 18:2188-2193, 1990) for imparting tolerance to sulfonylurea herbicides; polynucleotide molecules known as bar genes disclosed in DeBlock et al. (EMBO J. 6:2513-2519, 1987) for imparting glufosinate and bialaphos tolerance; polynucleotide molecules disclosed in U.S. Pat. Application Publication 2003/010609 A1 for imparting N-amino methyl phosphonic acid tolerance; polynucleotide molecules disclosed in U.S. Pat. No. 6,107,549 for imparting pyridine herbicide resistance; molecules and methods for imparting tolerance to multiple herbicides such as glyphosate, atrazine, ALS inhibitors, isoxoflutole and glufosinate herbicides are disclosed in U.S. Pat.t No. 6,376,754 and U.S. Pat. Application Publication 2002/0112260.

Genetic elements, methods, and transgenes that confer fungal disease resistance may also be used with the present disclosure (e.g., U.S. Pat. Nos. 6,653,280; 6,573,361; 6,506,962; 6,316,407; 6,215,048; 5,516,671; 5,773,696; 6,121,436; 6,316,407; 6,506,962).

### V. Definitions

The following definitions are provided to define and clarify the meaning of these terms in reference to the relevant embodiments of the present disclosure as used herein and to guide those of ordinary skill in the art in understanding the present disclosure. Unless otherwise noted, terms are to be understood according to their conventional meaning and usage in the relevant art, particularly in the field of molecular biology and plant transformation.

When introducing elements of the present disclosure or the embodiment(s) thereof, the articles “a”, “an”, “the”, and “said” are intended to mean that there are one or more of the elements.

The term “and/or”, when used in a list of two or more items, means any one of the items, any combination of the items, or all of the items with which this term is associated.

The terms “comprising”, “including”, and “having” are intended to be inclusive and mean that there may be additional elements other than the listed elements. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

As used herein, a “plant” includes a whole plant, explant, plant part, seedling, or plantlet at any stage of regeneration or development.

As used herein, a “plant part” can refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed, embryo, endosperm, seed coat, fruit, the mature ovary, propagule, or other plant tissues (e.g., vascular tissue, dermal tissue, ground tissue, and the like), or any portion thereof. Plant parts of the present disclosure can be viable, nonviable, regenerable, and/or non-regenerable. A “propagule” can include any plant part that can grow into an entire plant.

An “embryo” is a part of a plant seed, consisting of precursor tissues (e.g., meristematic tissue) that can develop into all or part of an adult plant. An “embryo” may further include a portion of a plant embryo.

A “meristem” or “meristematic tissue” comprises undifferentiated cells or meristematic cells, which are able to differentiate to produce one or more types of plant parts, tissues or structures, such as all or part of a shoot, stem, root, leaf, seed, etc.

As used herein, “genomic DNA” or “gDNA” refers to chromosomal DNA of an organism.

As used herein, a “genomic modification” (also referred to as “modification”) or “genomic edit” (also referred to as “edit”) refers to any modification to a genomic nucleotide sequence as compared to a wild-type or control plant. A genomic modification or genomic edit comprises a deletion, an insertion, a substitution, an inversion, a duplication, or any combination thereof.

As used herein, “T-DNA” or “transfer DNA” refers to the transferred DNA of the tumor-inducing (Ti) plasmid of some species of bacteria such as Agrobacterium tumefaciens.

As used herein, an “interfering protein” refers to a protein comprising an alteration that interferes with the normal activity of a protein lacking the alteration, such as a wild-type protein. Non-limiting examples of such interference include, reducing or disrupting the normal protein-protein interactions of the wild-type protein, binding protein-protein interaction partners in a non-functional manner, and/or forming non-functional protein complexes.

As used herein, a “dominant effect” refers to the phenomenon of one allele of a gene on a chromosome masking or overriding the effect of a different allele of the same gene on the other copy of the chromosome. A “dominant effect” may also refer to the observance of an allele associated phenotype in a plant that is heterozygous at the gene of interest.

As used herein, “number of kernels per ear” is a measure of the plot average of the number of kernels divided by the number of ears.

As used herein, “kernel rank” is a measure of the number of kernels per longitudinal row of ear.

As used herein, “ear area” is measured as the plot average of the area of an ear from a two-dimensional view by imaging the ear and including kernels and tip void in the area measurement.

As used herein, “ear length” is a measure of the plot average of the length of an ear measured from the tip of the ear in a straight line to the base of the ear node.

As used herein, “ear size related traits” refers to traits such as ear area, ear diameter, and ear length.

As used herein, “ear diameter” is a measure of the plot average of the ear diameter measured as the maximal “wide” axis of an ear over its widest section.

As used herein, “single kernel weight” is measured as the plot average of weight per kernel, calculated as the sample kernel weight (adjusted to a standard moisture level) / sample kernel number.

As used herein, “kernel row number” is the plot average of the number of rows of kernels on an ear, by counting around the circumference of the ear.

As used herein, “yield” refers to the amount of crop harvested per area of land. Non-limiting examples of yield measurements include “grain yield estimate” and “grain yield estimate per plant.” Grain yield estimate is a conversion from the hand-harvested grain weight per area measurement, collected from a small section of a plot, to the equivalent number of bushels per acre, including adjustment to a standard moisture level. Grain yield estimate per plant is calculated grain yield of a population of plants (ears) / number of plants (ears) sampled (unit is ounces).

“Standard agronomic practices” are known to those of skill in the art and refer to well-accepted methods and techniques for the cultivation and evaluation of crop species. For example, a field trial carried out under standard agronomic practices carefully controls for confounding factors including the environment, and thus the trial reflects the intrinsic morphology and physiology of the varieties being tested.

As used herein, the “vegetative phase” of plant development is the period of growth between germination and flowering. For maize, a common plant development scale used in the art is known as V-Stages. The V-stages are defined according to the uppermost leaf in which the leaf collar is visible. VE corresponds to emergence, V1 corresponds to first leaf, V2 corresponds to second leaf, V3 corresponds to third leaf, V(n) corresponds to nth leaf. VT occurs when the last branch of tassel is visible but before silks emerge. When staging a field of maize, each specific V-stage is defined only when 50 percent or more of the plants in the field are in or beyond that stage. Other development scales are known to those of skill in the art and may be used with the methods of the invention. The stages in the reproductive phase of corn are as follows R1 (silking; silks emerge from husks); R2 (blister; kernels are white on outside and inner fluid is clear); R3 (milk, kernels are yellow on the outside and inner fluid is milky-white); R4 (dough; milky inner fluid thickens from starch accumulation); R5 (dent; more than 50% of kernels are dented); and R6 (physiological maturity; black layer formed). Corn vegetative and reproductive stages are well known to those of skill in the art and numerous publications describing these stages can be found on the world wide web and elsewhere.

As used herein, the term “isogenic” means genetically uniform, whereas non-isogenic means genetically distinct.

All methods described herein can be performed in any suitable order unless otherwise indicated herein or clearly contradicted by context. The use of any and all examples, or exemplary language (e.g., “such as”) provided with respect to certain embodiments herein is intended merely to illuminate the present disclosure and does not pose a limitation on the scope of the present disclosure otherwise claimed.

### EXAMPLES

### Example 1. Design of the Gene Editing Constructs

As described herein, utilizing gene editing to perform targeted mutagenesis of the ubiquitin receptor gene ZmDA1 in corn offers a unique opportunity to modulate cell proliferation and increase corn yield per ear. The edited ZmDA1 gene can encode modified proteins with reduced, disrupted, or altered activity, e.g., peptidase activity. Additionally, such modified proteins are capable of interfering with protein-protein interactions and may produce a dominant effect on kernel number and ear size related traits and thereby increase yield in hybrid corn.

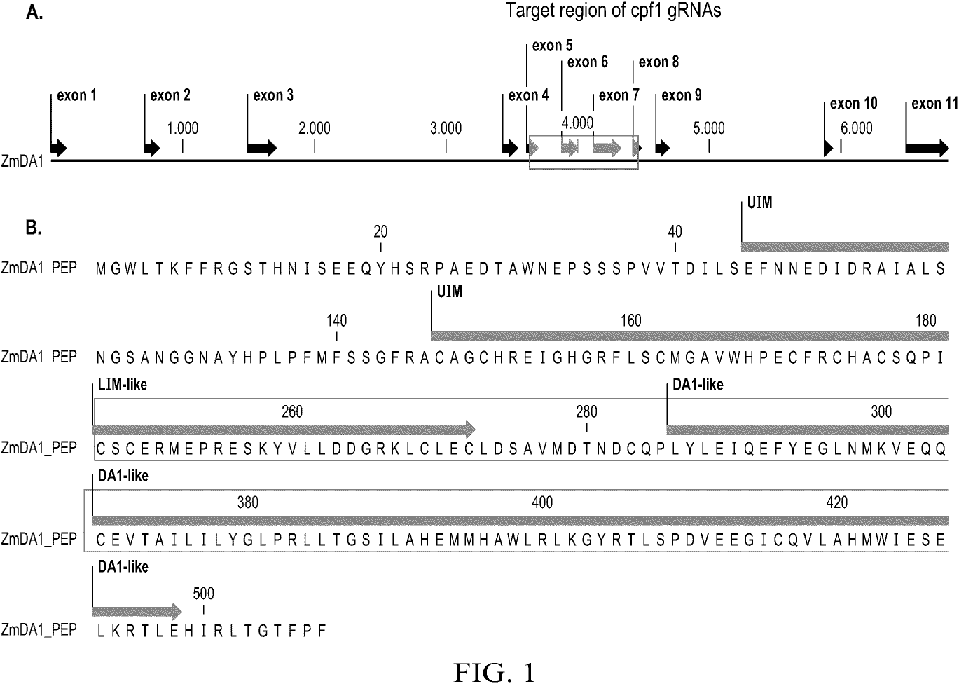

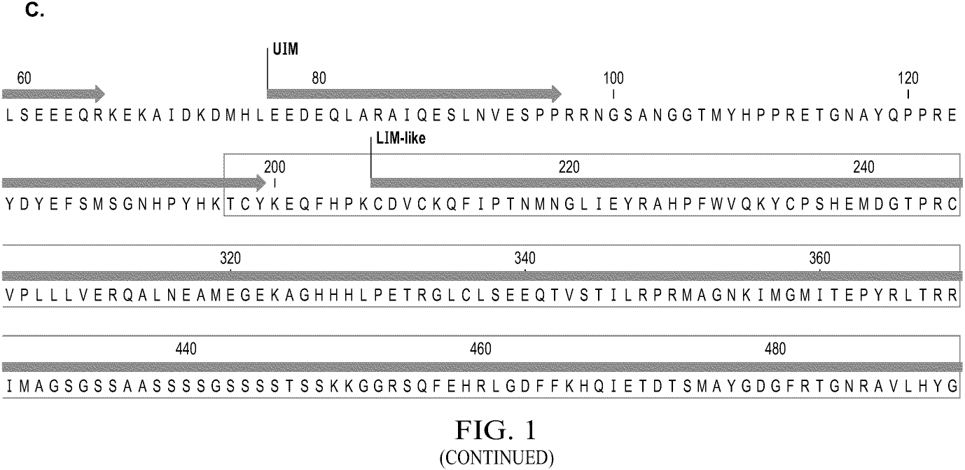

The coding sequence of the ZmDA1 gene is provided as SEQ ID NO:1, the amino acid sequence for the ZmDA1 protein is provided as SEQ ID NO:2, and the genomic DNA (gDNA) sequence for the ZmDA1 gene starting from the start codon of the first exon and ending at the stop codon in the last exon, including introns, is provided as SEQ ID NO:3, the coordinates of the exons are described in Table 1. Briefly, “gDNA” represents the ZmDA1 gene; “Exon Number” represents the exon described by the coordinates (visualized in FIG. 1); and “Coordinates” represents the nucleotide base location in the gDNA sequence.

TABLE 1

Exon coordinates for gDNA sequence of the ZmDA1 gene

| gDNA | Exon Number | Coordinates | SEQ ID NO |
|---|---|---|---|
| ZmDA1 | 1 | 1-120 | 3 |
| | 2 | 710-800 | |
| | 3 | 1490-1712 | |
| | 4 | 3430-3550 | |
| | 5 | 3615-3698 | |
| | 6 | 3880-3999 | |
| | 7 | 4120-4332 | |
| | 8 | 4421-4483 | |
| | 9 | 4591-4703 | |
| | 10 | 5873-5931 | |
| | 11 | 6492-6811 | |

The regions of the ZmDA1 gene coding for the LIM domain (SEQ ID NO:3 nucleotide bases 3434-3656), LIM-like domain (SEQ ID NO:3 nucleotide bases 3678-4176) or C-terminal DA1-like domain (SEQ ID NO:3 nucleotide bases 4216-6811) were targeted for mutagenesis by gene editing to produce interfering proteins. Three gene editing constructs for plant transformation were designed with multiple guide RNAs (gRNAs) targeting specific locations within a 785 bp region in the ZmDA1 gene beginning at 6 nucleotides inside of the 3′ end of the region coding for the LIM domain through the C-terminal DA1-like domain coding region, as illustrated in FIG. 1.

Each of the three gene editing constructs was designed to create double-stranded breaks (DSBs) at locations targeted by the gRNAs. Modifications by gRNAs include deletions at DSB sites with potential reading frame shifts, pre-mature stop sequences, or alternative splicing post-editing. Base changes or insertions are possible around the deletions.

In this example, the three gene editing constructs each comprised two to three functional regions or cassettes relevant to gene editing and the creation of the DSBs in the edited ZmDA1 gene regions: expression of a Cpfl protein and expression of one or two guide RNAs targeting the ZmDA1 gene sequence coding for the LIM domain, LIM-like domain, or C-terminal DA1-like region. Each guide RNA unit contains a common scaffold compatible with the Cpf1 gene (SEQ ID NO:4), and a unique spacer/targeting sequence complementary to an intended target site as listed in Table 2. Some of the constructs also comprised functional regions or cassettes relevant to gene editing and the creation of the DSBs in genomic regions outside of the ZmDA1 gene.

The Cpfl expression cassette of all constructs in Table 2 comprised a Zea mays polyubiquitin promoter (SEQ ID NO:5) operably linked to a sequence codon-optimized for corn encoding a Lachnospiraceae bacterium Cpfl RNA-guided endonuclease enzyme (SEQ ID NO:6) fused to two copies of a nuclear localization signal (SEQ ID NO:7) (See, e.g., Gao et al., Nature Biotechnol. 35(8):789-792, 2017; incorporated herein by reference in its entirety).

One type of gRNA expression cassette, present in construct pC1DA1, comprised a sequence encoding one ZmDA1 guide RNA operably linked to a plant-expressible promoter. Two such cassettes are present in pC1DA1 (guide RNA spacers DA1_g+4413 and DA1_g-4348). Spacer sequences as listed in Table 2 targeted alternative DSB sites coding for the LIM domain, LIM-like domain, or C-terminal DA1-like domain region of the ZmDA1 gene. Another type of gRNA expression cassette, present in constructs pC2DA1 (guide RNA spacers DA1_g+4413 and DA1_g-4348) and pC3DA1 (guide RNA spacers DA1_g-3673 and DA1_g-4348), comprised a sequence encoding two ZmDA1 guide RNAs operably linked to a plant-expressible promoter. Spacer sequences as listed in Table 2 targeted alternative DSB sites in the ZmDA1 gene coding for the LIM domain, the LIM-like domain, or C-terminal DA1-like domain. In sum, Table 2 shows example gRNAs used for editing the ZmDA1 gene in regions coding for the LIM domain, LIM-like domain, or C-terminal DA1-like domain.

TABLE 2

Example guide RNAs used for editing the endogenous ZmDA1 gene

| Construct | Guide RNA Spacer | Target Site | Spacer Sequence | SEQ ID NO: |
|---|---|---|---|---|
| pC1DA1 | DA1_g+4413 | gDNA: 4413...4435 | GATACCAGGGTCACCACCATCTT | 8 |
| | DA1_g-4348 | gDNA: 4326...4348 | AGCATAGTAAACATACAGCCTTC | 9 |
| pC2DA1 | DA1_g+4413 | gDNA: 4413...4435 | GATACCAGGGTCACCACCATCTT | 8 |
| | DA1_g-4348 | gDNA: 4326...4348 | AGCATAGTAAACATACAGCCTTC | 9 |
| pC3DA1 | DA1_g-3673 | gDNA: 3651...3673 | GGGTGAAACTGCTCCTTGTAGCA | 10 |
| | DA1_g-4348 | gDNA: 4326...4348 | AGCATAGTAAACATACAGCCTTC | 9 |

### Example 2. Confirmation of Edited Alleles of Plants Produced by the Gene Editing Constructs pC1DA1, pC2DA1, and pC3DA1

An inbred corn plant line (wild-type, or WT) was transformed via Agrobacterium-mediated transformation with one of the pC1DA1, pC2DA1, or pC3DA1 constructs as described above in Example 1. The transformed plant tissues were grown to produce mature plants and DNA sequencing was performed to screen for genomic modifications in the ZmDA1 gene as well as all other applicable genomic regions relevant to the construct used. Following one to two generations of self-crossing of edited plants, plants homozygous for edited alleles of the ZmDA1 edited region, devoid of editing T-DNA sequences, and also devoid of genomic modifications outside of the ZmDA1 gene were crossed with a different WT male corn plant line to produce hybrid plants.

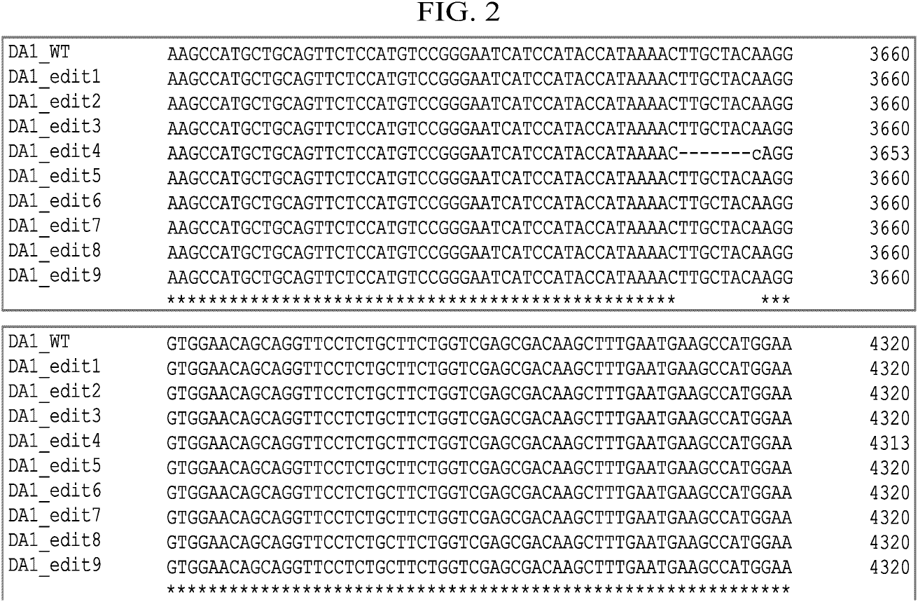

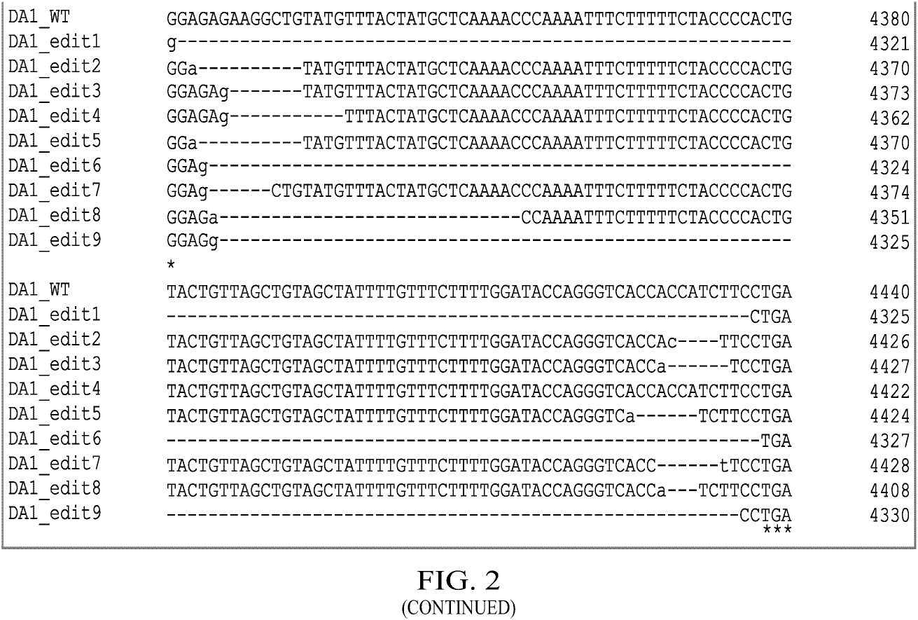

To determine the edit(s) in the ZmDA1 region coding for the LIM domain, LIM-like domain, and C-terminal DA1-like domain, an amplicon sequencing technique was used to produce sequences for each edited region for comparison with wild-type sequences. Amplicon sequencing consists of the generation of one or more unique PCR products across the genomic region of interest for further sequencing analysis, e.g., using Next-Gen Sequencing techniques known in the art. Sequence data from each sample is then mapped to a reference sequence to identify consensus differences. Plants with unique modifications were selected to provide diverse coverage of single gene mutations in the targeted genomic regions. Such plants were assayed for edited regions and lesions reported in Table 3 and FIG. 2.

Briefly, Table 3 summarizes edited plants produced by pC1DA1, pC2DA1, and pC3DA1 editing constructs with one or more edits to the ZmDA1 gene in regions coding for the LIM domain, LIM-like domain, or C-terminal DA1-like domain; “Editing Construct” refers to the construct transformed into the transformant plant to induce one or more edits to the ZmDA1 gene. “Edit Name” is the identifier for a unique edited plant produced (also referred to as “ZmDA1 gene edits” or “ZmDA1 edited plants”); Null_DA1 (WT) corresponds to the unedited, wild-type corn plant. “Causal Lesion(s)” indicates coordinates of the edited gene region including size in nucleotide base pairs. “Coordinates” in this connection represents the nucleotide base location in the gDNA sequence of the ZmDA1 wild-type gene (SEQ ID NO:3), not in the ZmDA1 gene edit(s) sequences (SEQ ID NO:11 to SEQ ID NO:19). Throughout Table 3 “NA” indicates that the identifier is not applicable to the WT plant.

TABLE 3

Edited plants produced by pC1DA1, pC2DA1, and pC3DA1 editing construct, with segmental modifications of the ZmDA1 gene

| Editing Construct | Edit Name | Causal Lesion(s) | SEQ ID NO: |
|---|---|---|---|
| NA | Null_DA1 (WT) | NA | 3 |
| pC1DA1 | DA1_edit1 | 4322...4436 (115 bp) | 11 |
| pC1DA1 | DA1_edit2 | 4324...4333 (10 bp); 4430...4433 (4 bp) | 12 |
| pC1DA1 | DA1_edit3 | 4327...4333 (7 bp); 4429...4434 (6 bp) | 13 |
| pC3DA1 | DA1_edit4 | 3650...3657 (8 bp); 4327...4337 (11 bp) | 14 |
| pC2DA1 | DA1_edit5 | 4324...4333 (10 bp); 4426...4431 (6 bp) | 15 |
| pC2DA1 | DA1_edit6 | 4325...4437 (113 bp) | 16 |
| pC2DA1 | DA1_edit7 | 4325...4330 (6 bp); 4428...4433 (6 bp) | 17 |
| pC2DA1 | DA1_edit8 | 4326...4354 (29 bp); 4429...4431 (3 bp) | 18 |
| pC2DA1 | DA1_edit9 | 4325...4435 (111 bp) | 19 |

FIG. 2 depicts the aligned positional sequence changes in the ZmDA1 gene edits described in Table 3. In FIG. 2, asterisks (*) indicate positions in which all edited sequences maintain the same base as the WT, lacking either base deletion or base change; and dashes (-) indicate base deletions compared to WT. Several ZmDA1 gene edits contain nucleotide substitutions or nucleotide insertions around the deleted region (DA1_edit4 and DA1_edit9). Most ZmDA1 gene edits consist of modifications causing changes to the C-terminal DA1-like domain of the ZmDA1 protein. DA1_edit4 begins near the end of the region coding for the LIM domain, causing a frame shift and premature stop codon.

### Example 3. Kernel/Ear/Yield Potential Estimates of Plants With Edited Alleles

A field trial was carried out under standard agronomic practices to evaluate key yield-related traits of hybrid plants comprising a ZmDA1 edited region and devoid of editing T-DNA sequences.

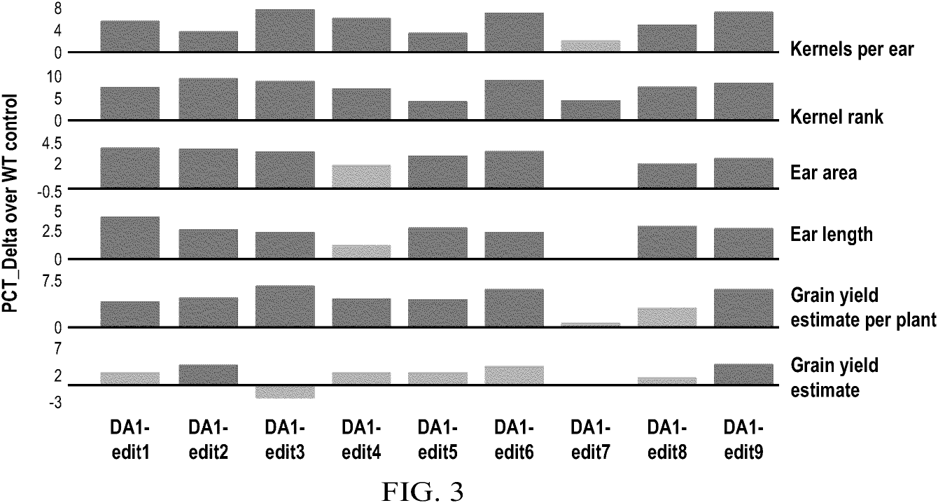

Corn ear traits were measured at the R6 stage. Ear size related traits were measured through imaging analysis. Results are shown in FIG. 3 as percent difference (delta) between edited plants and wild-type control plants. In FIG. 3, dark gray bars represent significant increase or decrease at P value less than 0.2; and light gray bars represent increase or decrease in yield related trait at P value 0.2 and above. The following traits were measured and reported in FIG. 3: number of kernels per ear; kernel rank; ear area; and ear length. Typically, 128 representative ears (approximately 16 replicates of 8 ears per replicate) were measured per ZmDA1 edited plant in the trial.

Grain yield estimate and grain yield estimate per plant were also determined and reported in FIG. 3. Grain yield estimate is a conversion from the hand-harvested grain weight per area measurement, collected from a small section of a plot, to the equivalent number of bushels per acre, including adjustment to a standard moisture level. Grain yield estimate per plant is calculated grain yield of a population of plants (ears) / number of plants (ears) sampled (unit is ounces).

As shown in FIG. 3, ZmDA1 edited plants exhibited statistically significant improvement in several yield-related traits, including increased kernels per ear, kernel rank, ear size related traits (ear area and ear length) and grain yield estimate per plant among most ZmDA1 edited plants in comparison to control plants. Grain yield estimate is also increased for some specific ZmDA1 edited plants.

The yield trait data presented in this example suggest that targeted editing of the ZmDA1 gene may lead to increased kernel number and ear size related traits compared to WT controls, suggesting that these genomic edits may produce a dominant effect on increased yield traits.

### Example 4: Design of the Gene Editing Constructs pC4DA1, pC5DA1, and pC6DA1

Three additional gene editing constructs were designed to produce novel edited allele variants in the ZmDA1 gene. All 3 constructs targeted the region of the ZmDA1 gene coding for the LIM domain (SEQ ID NO:3 nucleotide bases 3434-3656), LIM-like domain (SEQ ID NO:3 nucleotide bases 3678-4176), or C-terminal DA1-like domain (SEQ ID NO:3 nucleotide bases 4216-6811) for mutagenesis by gene editing. The three gene editing constructs for plant transformation were designed with two guide RNAs (gRNAs) each, DA1_g+4413 and DA1_g-4348 as described in Example 1.

In this example, the three gene editing constructs each comprised two functional regions or cassettes relevant to gene editing and the creation of the DSBs in the edited ZmDA1 gene regions: expression of a Cpf1 protein and expression of two guide RNAs targeting the ZmDA1 gene sequence coding for the LIM domain, the LIM-like domain, or C-terminal DA1-like domain region. Each guide RNA unit contains a common scaffold compatible with the Cpfl gene (SEQ ID NO:4), and a unique spacer/targeting sequence complementary to an intended target site as listed in Table 4. Some of the constructs also comprised functional regions or cassettes relevant to gene editing and the creation of the DSBs in genomic regions outside of the ZmDA1 gene (data not shown). The Cpfl expression cassette of all constructs in Table 4 comprised a Zea mays polyubiquitin promoter (SEQ ID NO:5) operably linked to a sequence codon-optimized for corn encoding a Lachnospiraceae bacterium Cpfl RNA-guided endonuclease enzyme (SEQ ID NO:6) fused to two copies of a nuclear localization signal (SEQ ID NO:7) (See, e.g., Gao et al., Nature Biotechnol. 35(8):789-792, 2017; incorporated herein by reference in its entirety).

The gRNA expression cassette, present in constructs pC4DA1, pC5DA1, and pC6DA1, comprises a sequence encoding two ZmDA1 guide RNAs operably linked to a plant-expressible promoter. Spacer sequences as listed in Table 4 targeted alternative DSB sites coding for the LIM domain, the LIM-like domain, or C-terminal DA1-like domain region of the ZmDA1 gene. All 3 constructs, pC4DA1, pC5DA1, and pC6DA1 comprise the same two guide RNA spacers: guide RNA spacers DA1_g+4413 and DA1_g-4348. In sum, Table 4 shows example gRNAs used for editing the ZmDA1 gene.

TABLE 4

Example guide RNAs used for editing the endogenous ZmDA1 gene

| Construct | Guide RNA Spacer | Target Site | Spacer Sequence | SEQ ID NO: |
|---|---|---|---|---|
| pC4DA1 | DA1_g+4413 | gDNA: 4413...4435 | GATACCAGGGTCACCACCATCTT | 8 |
| | DA1_g-4348 | gDNA: 4326...4348 | AGCATAGTAAACATACAGCCTTC | 9 |
| pC5DA1 | DA1_g+4413 | gDNA: 4413...4435 | GATACCAGGGTCACCACCATCTT | 8 |
| | DA1_g-4348 | gDNA: 4326...4348 | AGCATAGTAAACATACAGCCTTC | 9 |
| pC6DA1 | DA1_g+4413 | gDNA: 4413...4435 | GATACCAGGGTCACCACCATCTT | 8 |
| | DA1_g-4348 | gDNA: 4326...4348 | AGCATAGTAAACATACAGCCTTC | 9 |

### Example 5: Confirmation of Edited Alleles of Plants Produced by the Gene Editing Constructs pC4DA1, pC5DA1, and pC6DA1

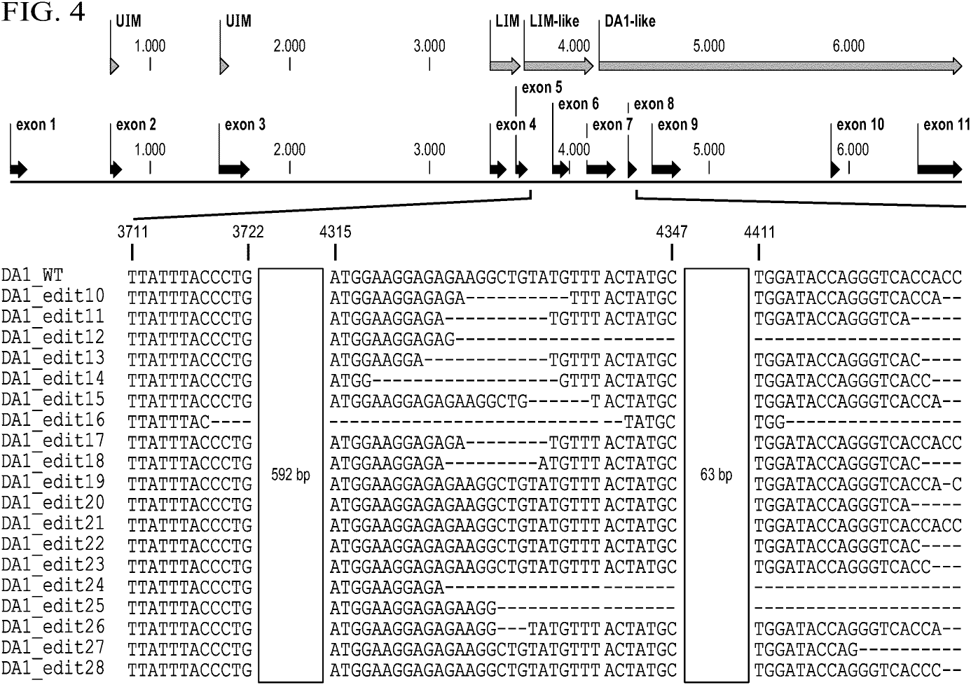

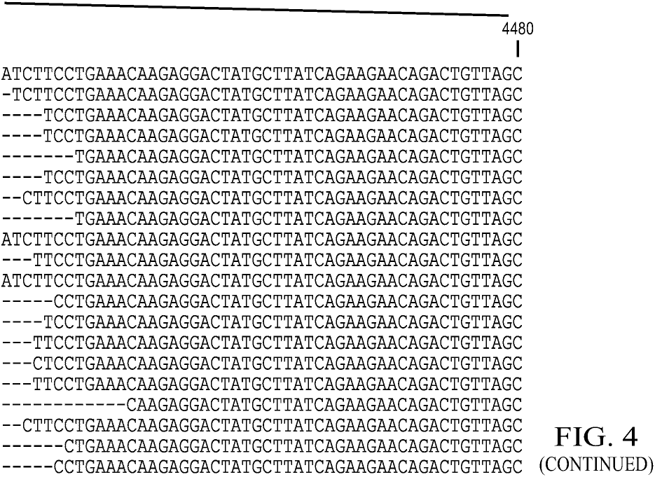

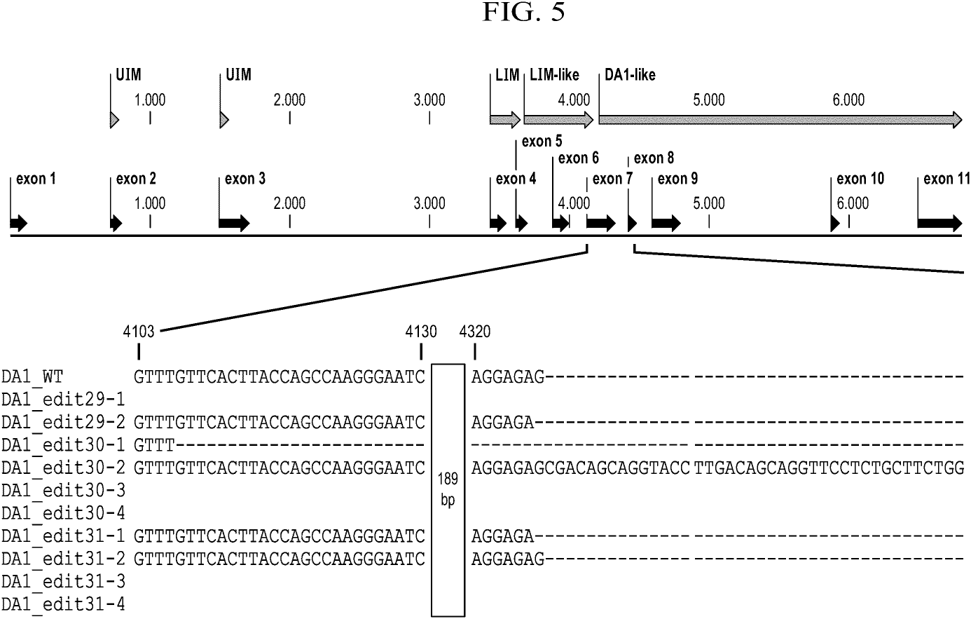

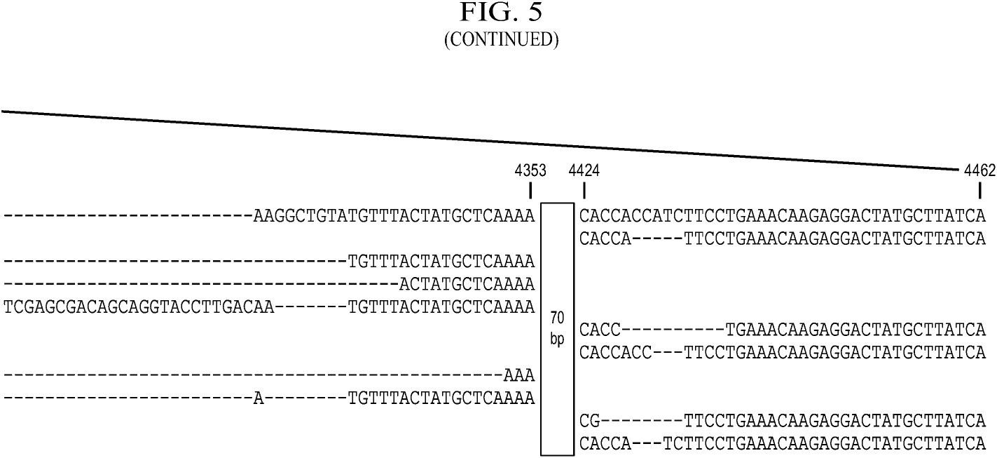

An inbred corn plant line (WT) was transformed via Agrobacterium-mediated transformation with one of the pC4DA1, pC5DA1, or pC6DA1 constructs as described in Example 2. The transformed plant tissues were grown to produce mature R₀ plants. Select R₀ plants having one or more unique genome edit(s) were self-crossed to produce homozygous R1 plants. To determine the edits in the endogenous ZmDA1 gene of the R₀ and R1 plants, an amplicon sequencing technique was performed. Edited R1 plants described in Table 5 and FIG. 4 were resultant of transformation with pC4DA1 or pC5DA1. Edits in R₀ plants described in Table 6 and FIG. 5 were resultant of transformation with pC6DA1.

Briefly, Table 5 summarizes homozygous edited R1 plants produced by pC4DA1 and pC5DA1 editing constructs with edit(s) to the ZmDA1 gene in the region coding for the LIM domain, the LIM-like domain, or C-terminal DA1-like domain. “Editing Construct” refers to the construct transformed into the transformant plant to induce one or more edits to the ZmDA1 gene. “Edit Name” is the identifier for a unique edited plant produced (also referred to as “ZmDA1 gene edits” or “ZmDA1 edited plants”); Null_DA1 (WT) corresponds to the unedited, wild-type corn plant. “Causal Lesion(s)” indicates coordinates of the edited gene region including size in nucleotide base pairs. “Coordinates” in this connection represents the nucleotide base location in the gDNA sequence of the ZmDA1 wild-type gene (SEQ ID NO:3), not in the ZmDA1 gene edit(s) sequences (SEQ ID NO:20 to SEQ ID NO:38). “NA” indicates that the identifier is not applicable to the WT plant. The coordinates presented in Table 5 and FIG. 4 are based on an alignment created using the MUSCLE (Multiple Sequence Comparison by Log-Expectation) computational alignment method. Visibly misaligned bases at either end of a modification, an occasional byproduct of multiple sequence alignment methods, were corrected manually so that the coordinates reflect the correct positions of the modifications with reference to SEQ ID NO:3. In some edits, multiple sets of “Causal Lesion” coordinates are possible as depending on the alignment method utilized, the coordinates may differ due to presence of identical base(s) at either end of a modification. For example, depending on alignment method utilized, the 1 bp deletion comprised in DA1_edit19 can be located either at bp position 4430 or bp position 4429 with reference to SEQ ID NO:3, depending on the bp position of the “C” nucleotide allocated by the computational alignment method. However, regardless of the alignment method utilized, all edits are unambiguously defined by their individual sequence presented in Table 6 via the identifier “SEQ ID NO:”.

TABLE 5

Edited homozygous R1 plants produced by pC4DA1 and pC5DA1 editing constructs, with segmental modifications of the ZmDA1 gene

| Editing Construct | Edit Name | Causal Lesion(s) | SEQ ID NO: |
|---|---|---|---|
| NA | Null_DA1 (WT) | NA | 3 |
| pC4DA1 | DA1_edit10 | 4328...4337 (10 bp); 4429...4431 (3 bp) | 20 |
| pC4DA1 | DA1_edit11 | 4326...4335(10 bp); 4426...4434 (9 bp) | 21 |
| pC4DA1 | DA1_edit12 | 4327...4434 (108 bp) | 22 |
| pC4DA1 | DA1_edit13 | 4324...4335(12 bp); 4427...4437 (11 bp) | 23 |
| pC4DA1 | DA1_edit14 | 4319...4336 (18 bp); 4428...4434 (7 bp) | 24 |
| pC4DA1 | DA1_edit15 | 4334...4339 (6 bp); 4429...4432 (4 bp) | 25 |
| pC4DA1 | DA1_edit16 | 3719...4342 (624 bp); 4414...4437 (24 bp) | 26 |
| pC4DA1 | DA1_edit17 | 4328...4335 (8 bp) | 27 |
| pC4DA1 | DA1_edit18 | 4326...4334 (9 bp); 4427...4433 (7 bp) | 28 |
| pC4DA1 | DA1_edit19 | 4429 (1 bp) | 29 |
| pC4DA1; pC5DA1 | DA1_edit20 | 4426...4435 (10 bp) | 30 |
| pC4DA1 | DA1_edit21 | 4431...4434 (4 bp) | 31 |
| pC4DA1 | DA1_edit22 | 4427...4433 (7 bp) | 32 |
| pC4DA1 | DA1_edit23 | 4428...4434 (7 bp modification including 1 bp substitution/insertion) | 33 |
| pC5DA1 | DA1_edit24 | 4326...4433 (108 bp) | 34 |
| pC5DA1 | DA1_edit25 | 4331...4442 (112 bp) | 35 |
| pC5DA1 | DA1_edit26 | 4331...4333 (3 bp); 4429...4432 (4 bp) | 36 |
| pC5DA1 | DA1_edit27 | 4421...4436 (16 bp) | 37 |
| pC5DA1 | DA1_edit28 | 4428...4435 (8 bp modification including 1 bp substitution/insertion) | 38 |

DA1_edit20 (SEQ ID NO:30) was produced individually by both constructs pC4DA1 and pC5DA1 in separate plants, producing two plants carrying an identical edit. As such, they are listed in the same row of Table 5. FIG. 4 schematically depicts the aligned positional sequence changes in the ZmDA1 gene edits described in Table 5. ZmDA1 edits have deletions that may lead to early translation termination, reading frame shifts, or splice-site disruptions.

Briefly, Table 6 summarizes the modifications detected in edited R₀ plants produced by pC6DA1 editing constructs with edit(s) to the ZmDA1 gene in the region coding for the LIM domain, the LIM-like domain, or C-terminal DA1-like domain. In the R₀ plants, the zygosity of the edited alleles of the ZmDA1 gene was not determined, therefore the alleles of the ZmDA1 gene comprising edits 29-31 in edited R₀ plants can be heterozygous and/or biallelic. “Editing Construct” refers to the construct transformed into the transformant plant to induce one or more edits to the ZmDA1 gene. “Edit Allele Name” is the identifier for a unique edited R₀ plant produced. “Modification Number” presents the identifier assigned to that lesion/modification in the said unique edited plant. “Causal Lesion(s)” indicates coordinates of the edited gene region including size in nucleotide base pairs. “Coordinates” in this connection represents the nucleotide base location in the gDNA sequence of the ZmDA1 wild-type gene (SEQ ID NO:3), not in the ZmDA1 gene edit(s) sequences (SEQ ID NO:50 to SEQ ID NO:59). Throughout Table 6 “NA” indicates that the identifier is not applicable to the WT plant. The coordinates presented in Table 6 and FIG. 5 are based on an alignment created using the MUSCLE (Multiple Sequence Comparison by Log-Expectation) computational alignment method. Visibly misaligned bases at either end of a modification, an occasional byproduct of multiple sequence alignment methods, were corrected manually so that the coordinates reflect the correct positions of the modifications with reference to SEQ ID NO:3. In some edits, multiple sets of “Causal Lesion” coordinates are possible as depending on alignment method utilized, the coordinates may differ due to presence of identical base(s) at either end of a modification. For example, depending on alignment method utilized, the 10 bp deletion comprised in DA1_edit29 modification 2 can be located either from bp position 4325 to 4334 or from bp position 4326 to 4335 with reference to SEQ ID NO:3, depending on the bp position of the “A” nucleotide allocated by the computational alignment method. However, regardless of the alignment method utilized, all edits are unambiguously defined by their individual sequence presented in Table 6 via the identifier “SEQ ID NO:”. The SEQ ID NO: provided in Table 6 (excluding the WT plant SEQ ID NO: 3) references the sequence results of the amplicon sequencing technique used to detect the modifications (amplicons).

TABLE 6

Description of modifications derived from R₀ plants transformed with the pC6DA1 editing construct, with segmental modifications of the ZmDA1 gene

| Editing Construct | Edit Allele Name | Modification Number | Causal Lesion(s) | SEQ ID NO: |
|---|---|---|---|---|
| NA | Null_DA1 (WT) | NA | NA | 3 |
| pC6DA1 | DA1_edit29 | 1 | 4429...4433 (5 bp) | 50 |
| | | 2 | 4325...4334 (10 bp) | 51 |
| pC6DA1 | DA1_edit30 | 1 | 4107...4340 (234 bp) | 52 |
| | | 2 | 4327...4335 (7 bp deletion plus extra 64 bp insertion) | 53 |
| | | 3 | 4431...4433 (3 bp) | 54 |
| | | 4 | 4428...4437 (10 bp) | 55 |
| pC6DA1 | DA1_edit31 | 1 | 4326...4350 (25 bp) | 56 |
| | | 2 | 4328...4335 (8 bp) | 57 |
| | | 3 | 4425...4433 (9 bp modification including 1 bp substitution/insertion) | 58 |
| | | 4 | 4429...4431 (3 bp) | 59 |

FIG. 5 schematically depicts the aligned positional sequence changes in the ZmDA1 modifications described in Table 6. To allow unambigous allocation of the modifications depicted in FIG. 5 in accordinace with their description in Table 6, the “Edit Allele Name” and “Modification Number” have been combined in FIG. 5 to identify the sequence origin (for example “DA1_edit29-1” refers to Modification Number 1 in the Edit Allele “DA1_edit29” in Table 6). ZmDA1 gene edits described have modifications that may lead to early translation termination, reading frame shifts, or splice-site disruptions.

Of the R₀ plants produced by the pC6DA1 editing construct with edit(s) to the ZmDA1 gene, modified plants can be selected for continued analysis based on comprising one or more modifications as summarized in Table 6. These R₀ plants can comprise a combination of one or more modifications described in Table 6, depending on the actual zygosity of the alleles of the ZmDA1 gene in said R₀ plants. For example, plants derived from Edit Allele “DA1_edit30” may comprise alleles carrying a combination of one or more of DA1_edit30 Modification Numbers 1 to 4 described in Table 6, such as the alleles exemplified in Table 7. To obtain plants homozygous for specific edited alleles, R₀ plants produced by the pC6DA1 editing construct can be self-crossed for one or more generations and subsequently be tested for zygosity using any known zygosity assay that allows for the distinction between heterozygous, homozygous, and wild-type plants. Table 7 summarizes such potential homozygous edited plants obtainable from the R₀ plants produced by the pC6DA1 editing construct with edit(s) to the ZmDA1 gene. “Edit Allele Name in R₀ Plant” is the identifier for a unique edited R₀ plant produced by the pC6DA1 editing construct. “Edit Name of potential edited homozygous Plant” is the identifier for a potential unique edited plant obtainable from the R₀ plants produced by the pC6DA1 editing construct; Null_DA1 (WT) corresponds to the unedited, wild-type corn plant. “Causal Lesion(s)” indicates coordinates of the edited gene region in R₀ plants produced by the pC6DA1 editing construct including size in nucleotide base pairs. As in Table 5 and 6, “Coordinates” in this connection represents the nucleotide base location in the gDNA sequence of the ZmDA1 wild-type gene (SEQ ID NO:3). “SEQ ID NO: (in potential edited homozygous Plant)” refers to a potential edited ZmDA1 allele present in a plant obtainable from R₀ plants produced by the pC6DA1 editing construct. “SEQ ID NO: (in underlying Amplicon of generated R₀ Plant)” references the sequence results of the amplicon sequencing technique used to detect the modifications (amplicons) in R₀ plants produced by the pC6DA1 editing construct.

TABLE 7

Potential edited homozygous plants obtainable from R₀ plants produced by pC6DA1 diting construct, with segmental modifications of the ZmDA1 gene

| Edit Allele Name in R₀ Plant | Edit Name of potential edited homozygous Plant | Causal Lesion(s) | SEQ ID NO: (in potential edited homozygous Plant) | SEQ ID NO: (in underlying Amplicon of generated R₀ Plant) |
|---|---|---|---|---|
| | Null_DA1 WT | NA | 3 | NA |
| DA1_edit29 | DA1_edit29a | 4429...4433 (5 bp) | 39 | 50 |
| | DA1_edit29b | 4325...4334 (10 bp) | 40 | 51 |
| | DA1_edit29c | 4325...4334 (10 bp); 4429...4433 (5 bp) | 41 | 50 and 51 |
| DA1_edit30 | DA1_edit30a | 4107...4340 (234 bp); 4428...4437 (10 bp) | 42 | 52 and 55 |
| | DA1_edit30b | 4327...4335 (7 bp deletion plus extra 64 bp insertion); 4431...4433 (3 bp) | 43 | 53 and 54 |
| | DA1_edit30c | 4107...4340 (234 bp); 4431...4433 (3 bp) | 44 | 52 and 54 |
| | DA1_edit30d | 4327...4335 (7 bp deletion plus extra 64 bp insertion); 4428...4437 (10 bp) | 45 | 53 and 55 |
| DA1_edit31 | DA1_edit31a | 4326...4350 (25 bp); 4429...4431 (3 bp) | 46 | 56 and 59 |
| | DA1_edit31b | 4328...4335 (8 bp); 4425...4433 (9 bp modification including 1 bp substitution/insertion) | 47 | 57 and 58 |
| | DA1_edit31c | 4326...4350 (25 bp); 4425...4433 (9 bp modification including 1 bp substitution/insertion) | 48 | 56 and 58 |
| | DA1_edit31d | 4328...4335 (8 bp); 4429...4431 (3 bp) | 49 | 57 and 59 |

Modified plants may be further self-crossed or crossed to other plants to produce modified plant seeds and progeny. Such plant seeds and progeny can be hybrid seeds or plants. Field trials (e.g., as described in Example 3) can be carried out under standard agronomic practices to evaluate key yield-related traits of modified plants comprising a ZmDA1 edited region produced by editing construct pC4DA1, pC5DA1, and/or pC6DA1. It is expected that such plants have comparable high yield corn characteristics as previously described modified plants comprising modifications in the ZmDA1 gene produced by editing constructs pC1DA1, pC2DA1, or pC3DA1 (see Examples 2 and 3).

### EMBODIMENTS

For further illustration, additional non-limiting embodiments of the present invention are set forth below.

Embodiment A1 is a modified corn plant, corn plant seed, corn plant part, or corn plant cell, comprising a genomic modification that reduces or disrupts the activity of ZmDA1, as compared to the activity of ZmDA1 in an otherwise identical corn plant, corn plant seed, corn plant part, or corn plant cell that lacks the modification.

Embodiment A2 is the modified plant, plant seed, plant part, or plant cell of embodiment A1, wherein the modification is present in at least one allele of an endogenous ZmDA1 gene.

Embodiment A3 is the modified plant, plant seed, plant part, or plant cell of embodiment A1 or A2, wherein the genomic modification is in an endogenous ZmDA1 gene encoding a protein having at least 70%, at least 75%, at least 80%, at least 85%, at least 90%, at least 95%, at least 96%, at least 97%, at least 98%, at least 99%, at least 99.5%, or 100% sequence identity to SEQ ID NO:2.

Embodiment A4 is the modified plant, plant seed, plant part, or plant cell of embodiment A2 or A3, wherein the modification is in a transcribable region of the ZmDA1 gene.

Embodiment A5 is the modified plant, plant seed, plant part, or plant cell of any one of embodiments A2 to A4, wherein the modification is in a region of said ZmDA1 gene comprising a coding sequence for a LIM domain.

Embodiment A6 is the modified plant, plant seed, plant part, or plant cell of any one of embodiments A2 to A4, wherein the modification is in a region of said ZmDA1 gene comprising a coding sequence for a LIM-like domain.

Embodiment A7 is the modified plant, plant seed, plant part, or plant cell of any one of embodiments A2 to A4, wherein the modification is in a region of said ZmDA1 gene comprising a coding sequence for a C-terminal DA1-like domain.

Embodiment A8 is the modified plant, plant seed, plant part, or plant cell of any one of embodiments A4 to A7, wherein the modification is in an exon region of said ZmDA1 gene.

Embodiment A9 is the modified plant, plant seed, plant part, or plant cell of any one of embodiments A4 to A7, wherein the modification is in an intron region of said ZmDA1 gene.

Embodiment A10 is the modified plant, plant seed, plant part, or plant cell of any one of embodiments A4 to A7, wherein the modification is in an exon region and an intron region of said ZmDA1 gene.

Embodiment A11 is the modified plant, plant seed, plant part, or plant cell of any one of embodiments A1 to A10, wherein the plant, plant seed, plant part, or plant cell is heterozygous for the modification.

Embodiment A12 is the modified plant, plant seed, plant part, or plant cell of any one of embodiments A1 to A10, wherein the plant, plant seed, plant part, or plant cell is homozygous for the modification.

Embodiment A13 is the modified plant, plant seed, plant part, or plant cell of any one of embodiments A1 to A11, wherein the plant, plant seed, plant part, or plant cell comprises a first modification in a first allele of the ZmDA1 gene and a second modification in a second allele of the ZmDA1 gene, the first modification and the second modification being different from one another.

Embodiment A14 is the modified plant, plant seed, plant part, or plant cell of any one of embodiments A1 to A13, wherein the modification comprises a deletion, an insertion, a substitution, an inversion, a duplication, or a combination of any thereof.

Embodiment A15 is the modified plant, plant seed, plant part, or plant cell of embodiment 14, wherein the modification is located at about 2375 nucleotides or more upstream from the 3′ end of reference sequence SEQ ID NO:3.

Embodiment A16 is the modified plant, plant seed, plant part, or plant cell of embodiment 1-15, wherein the modification comprises a deletion, a substitution, an insertion, or a combination of any thereof.

Embodiment A17 is the modified plant, plant seed, plant part, or plant cell of any one of embodiments A1 to A16 wherein the plant, plant seed, plant part, or plant cell comprises a modification in at least one allele of the ZmDA1 gene, wherein the modification is selected from the group consisting of:
a 115 base pair deletion from nucleotide 4322 to nucleotide 4436, as compared to reference sequence SEQ ID NO:3;
a 10 base pair deletion from nucleotide 4324 to nucleotide 4333, as compared to reference sequence SEQ ID NO:3;
a 4 base pair deletion from nucleotide 4430 to nucleotide 4433, as compared to reference sequence SEQ ID NO:3;
a 7 base pair deletion from nucleotide 4327 to nucleotide 4333, as compared to reference sequence SEQ ID NO:3;
a 6 base pair deletion from nucleotide 4429 to nucleotide 4434, as compared to reference sequence SEQ ID NO:3;
an 8 base pair modification from nucleotide 3650 to nucleotide 3657, as compared to reference sequence SEQ ID NO:3;
a 11 base pair deletion from nucleotide 4327 to nucleotide 4337, as compared to reference sequence SEQ ID NO:3;
a 10 base pair deletion from nucleotide 4324 to nucleotide 4333, as compared to reference sequence SEQ ID NO:3;
a 6 base pair deletion from nucleotide 4426 to nucleotide 4431, as compared to reference sequence SEQ ID NO:3;
a 113 base pair deletion from nucleotide 4325 to nucleotide 4437, as compared to reference sequence SEQ ID NO:3;
a 6 base pair deletion from nucleotide 4325 to nucleotide 4330, as compared to reference sequence SEQ ID NO:3;
a 6 base pair deletion from nucleotide 4428 to nucleotide 4433, as compared to reference sequence SEQ ID NO:3;
a 29 base pair deletion from nucleotide 4326 to nucleotide 4354, as compared to reference sequence SEQ ID NO:3;
a 3 base pair deletion from nucleotide 4429 to nucleotide 4431, as compared to reference sequence SEQ ID NO:3;
a 111 base pair modification from nucleotide 4325 to nucleotide 4435, as compared to reference sequence SEQ ID NO:3; and
combinations of any thereof.

Embodiment A18 is the modified plant, plant seed, plant part, or plant cell of any one of embodiments A1 to A17, wherein the plant, plant seed, plant part, or plant cell comprises a modification in at least one allele of the ZmDA1 gene, wherein the modification is comprised within a genomic region between nucleotide positions 3434 and 6811 of reference sequence SEQ ID NO:3.

Embodiment A19 is the modified plant, plant seed, plant part, or plant cell of any one of embodiments A1 to A18, wherein the plant, plant seed, plant part, or plant cell comprises a modification in at least one allele of the ZmDA1 gene, wherein the modification is comprised within a genomic region between nucleotide positions 3650 and 4437 of reference sequence SEQ ID NO:3.

Embodiment A20 is the modified plant, plant seed, plant part, or plant cell of embodiment A18 or A19, wherein the modification comprises a deletion of at least 1, at least 3, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, or at least 150 consecutive nucleotides.

Embodiment A21 is the modified plant, plant seed, plant part, or plant cell of any one of embodiments A14 to A20, wherein the plant, plant seed, plant part, or plant cell comprises a chromosomal sequence in the ZmDA1 gene that has at least 70%, at least 75%, at least 80%, at least 85%, at least 90%, at least 95%, at least 96%, at least 97%, at least 98%, at least 99%, at least 99.5%, or 100% sequence identity to SEQ ID NO:3 in the regions outside of the deletion, the insertion, the substitution, the inversion, or the duplication.

Embodiment A22 is the modified plant, plant seed, plant part, or plant cell of any one of embodiments A1 to A21, wherein the plant, plant seed, plant part, or plant cell comprises a polynucleotide sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19.

Embodiment A23 is the modified plant, plant seed, plant part, or plant cell of any one of embodiments A1 to A22, wherein the modification disrupts or alters the activity of ZmDA1, as compared to the activity of ZmDA1 in an otherwise identical plant, plant seed, plant part, or plant cell that lacks the modification.

Embodiment A24 is the modified plant, plant seed, plant part, or plant cell of embodiment A23, wherein the modification alters peptidase activity of ZmDA1.

Embodiment A25 is the modified plant, plant seed, plant part, or plant cell of any one of embodiments A1 to A24, wherein the modification confers an altered phenotype to the plant, as compared to the phenotype of an otherwise identical plant that lacks the modification.

Embodiment A26 is the modified plant, plant seed, plant part, or plant cell of embodiment A25, wherein the altered phenotype comprises an increase in number of kernels per ear, number of kernels per longitudinal row of ear, ear area, ear length, yield, grain yield estimate per plant, grain yield estimate, or combinations of any thereof, as compared to the phenotype of an otherwise identical plant that lacks the modification.

Embodiment A27 is the modified plant, plant seed, plant part, or plant cell of any one of embodiments A1 to A26, wherein the modified plant exhibits increased yield, grain yield estimate per plant, grain yield estimate, or combinations of any thereof, as compared to an otherwise identical plant that lacks the modification.

Embodiment A28 is a polynucleotide sequence comprising a sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19.

Embodiment A29 is a guide RNA comprising a polynucleotide sequence selected from the group consisting of SEQ ID NO:8, SEQ ID NO:9, SEQ ID NO:10.

Embodiment A30 is a method for producing a corn plant comprising a modified ZmDA1 gene, the method comprising:
a) introducing a modification into at least one target site in an endogenous ZmDA1 gene of a corn plant cell that reduces or disrupts the activity of ZmDA1;
b) identifying and selecting one or more corn plant cells of step (a) comprising said modification in said ZmDA1 gene; and
c) regenerating at least a first plant from said one or more cells selected in step (b) or a descendent thereof comprising said modification.

Embodiment A31 is the method of embodiment A30, wherein the target site is located in a coding or non-coding region of said endogenous ZmDA1 gene.

Embodiment A32 is the method of embodiment A30, wherein the modification is in a region of said ZmDA1 gene comprising a coding sequence for a LIM domain.

Embodiment A33 is the method of embodiment A30, wherein the modification is in a region of said ZmDA1 gene comprising a coding sequence for a LIM-like domain.

Embodiment A34 is the method of embodiment A30, wherein the modification is in a region of said ZmDA1 gene comprising a coding sequence for a C-terminal DA1-like region.

Embodiment A35 is the method of embodiment A30, wherein introducing the modification comprises use of at least one site-specific genome modification enzyme in said plant cell.

Embodiment A36 is the method of embodiment A35, wherein the site-specific genome modification enzyme is selected from the group consisting of: an RNA-guided nuclease, a zinc-finger nuclease, a meganuclease, a TALE-nuclease, a recombinase, a transposase, and combinations of any thereof.

Embodiment A37 is the method of any one of embodiments A35 to A36, wherein the site-specific genome modification enzyme is an RNA-guided nuclease comprising a Cas nuclease, a Cpf1 nuclease, or a variant of either thereof.

Embodiment A38 is the method of any one of embodiments A35 to A37, wherein the site-specific genome modification enzyme creates at least one strand break at the target site.

Embodiment A39 is the method of embodiment A30, wherein the modification is selected from the group consisting of a substitution, an insertion, an inversion, a deletion, a duplication, and a combination thereof.

Embodiment A40 is the method of embodiment A39, wherein the modification is a deletion, substitution, insertion, or a combination thereof.

Embodiment A41 is the method of embodiment A40, wherein the deletion comprises a region of at least 1, at least 3, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, or at least 150 consecutive nucleotides.

Embodiment A42 is a method for producing a hybrid corn plant comprising a modified ZmDA1 gene, the method comprising crossing a corn plant comprising a modified ZmDA1 gene with a second, non-isogenic corn plant to produce a F₁ hybrid corn plant, wherein the modified ZmDA1 gene confers an altered phenotype to the hybrid corn plant as compared to the phenotype of an otherwise isogenic hybrid corn plant that lacks the modification.

Embodiment A43 is the method of embodiment A42 wherein the second, non-isogenic corn plant lacks a modified ZmDA1 gene.

Embodiment A44 is a modified plant, plant seed, plant part, or plant cell, wherein the plant, plant seed, plant part, or plant cell comprises a polynucleotide sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19.

Embodiment B1 is a modified corn plant, corn plant seed, corn plant part, or corn plant cell, comprising a genomic modification that reduces or disrupts the activity of ZmDA1, as compared to the activity of ZmDA1 in an otherwise identical corn plant, corn plant seed, corn plant part, or corn plant cell that lacks the modification.

Embodiment B2 is the modified plant, plant seed, plant part, or plant cell of embodiment B1, wherein the modification is present in at least one allele of an endogenous ZmDA1 gene.

Embodiment B3 is the modified plant, plant seed, plant part, or plant cell of embodiment B1 or B2, wherein the genomic modification is in an endogenous ZmDA1 gene encoding a protein having at least 70%, at least 75%, at least 80%, at least 85%, at least 90%, at least 95%, at least 96%, at least 97%, at least 98%, at least 99%, at least 99.5%, or 100% sequence identity to SEQ ID NO:2.

Embodiment B4 is the modified plant, plant seed, plant part, or plant cell of embodiment B2 or B3, wherein the modification is in a transcribable region of the ZmDA1 gene.

Embodiment B5 is the modified plant, plant seed, plant part, or plant cell of any one of embodiments B2 to B4, wherein the modification is in a region of said ZmDA1 gene comprising a coding sequence for a LIM domain.

Embodiment B6 is the modified plant, plant seed, plant part, or plant cell of any one of embodiments B2 to B4, wherein the modification is in a region of said ZmDA1 gene comprising a coding sequence for a LIM-like domain.

Embodiment B7-1 is the modified plant, plant seed, plant part, or plant cell of any one of embodiments B2 to B4, wherein the modification is in a region of said ZmDA1 gene comprising a coding sequence for a C-terminal DA1-like domain.

Embodiment B7-2 is the modified plant, plant seed, plant part, or plant cell of any one of embodiments B2 to B4, wherein the modification is in a region of said ZmDA1 gene comprising a coding sequence for a LIM-domain, and/or a LIM-like domain and/or a C-terminal DA1-like domain or a combination of any thereof.

Embodiment B8 is the modified plant, plant seed, plant part, or plant cell of any one of embodiments B4 to B7, wherein the modification is in an exon region of said ZmDA1 gene.

Embodiment B9 is the modified plant, plant seed, plant part, or plant cell of any one of embodiments B4 to B7, wherein the modification is in an intron region of said ZmDA1 gene.

Embodiment B10 is the modified plant, plant seed, plant part, or plant cell of any one of embodiments B4 to B7, wherein the modification is in an exon region and an intron region of said ZmDA1 gene.

Embodiment B11 is the modified plant, plant seed, plant part, or plant cell of any one of embodiments B1 to B10, wherein the plant, plant seed, plant part, or plant cell is heterozygous for the modification.

Embodiment B12 is the modified plant, plant seed, plant part, or plant cell of any one of embodiments B1 to B10, wherein the plant, plant seed, plant part, or plant cell is homozygous for the modification.

Embodiment B13 is the modified plant, plant seed, plant part, or plant cell of any one of embodiments B1 to B11, wherein the plant, plant seed, plant part, or plant cell comprises a first modification in a first allele of the ZmDA1 gene and a second modification in a second allele of the ZmDA1 gene, the first modification and the second modification being different from one another.

Embodiment B14 is the modified plant, plant seed, plant part, or plant cell of any one of embodiments B1 to B13, wherein the modification comprises a deletion, an insertion, a substitution, an inversion, a duplication, or a combination of any thereof.

Embodiment B15-1 is the modified plant, plant seed, plant part, or plant cell of embodiment B14, wherein the modification is located at about 2375 nucleotides or more upstream from the 3′ end of reference sequence SEQ ID NO:3.

Embodiment B15-2 is the modified plant, plant seed, plant part, or plant cell of embodiment B14, wherein the modification is located at about 2369 nucleotides or more upstream from the 3′ end of reference sequence SEQ ID NO:3.

Embodiment B16 is the modified plant, plant seed, plant part, or plant cell of embodiment B1- to B15-2, wherein the modification comprises a deletion, a substitution, an insertion, or a combination of any thereof.

Embodiment B17 is the modified plant, plant seed, plant part, or plant cell of any one of embodiments B1 to B16 wherein the plant, plant seed, plant part, or plant cell comprises a modification in at least one allele of the ZmDA1 gene, wherein the modification is selected from the group consisting of:
a 115 base pair deletion wherein the resulting nucleotide sequence is SEQ ID NO:11;
a 10 base pair deletion and a 4 base pair deletion wherein the resulting nucleotide sequence is SEQ ID NO:12;
a 7 base pair deletion and a 6 base pair deletion wherein the resulting nucleotide sequence is SEQ ID NO:13;
an 8 base pair modification and an 11 base pair deletion wherein the resulting nucleotide sequence is SEQ ID NO:14;
a 10 base pair deletion and a 6 base pair deletion wherein the resulting nucleotide sequence is SEQ ID NO:15;
a 113 base pair deletion wherein the resulting nucleotide sequence is SEQ ID NO:16;
a first 6 base pair deletion and a second 6 base pair deletion wherein the resulting nucleotide sequence is SEQ ID NO:17;
a 29 base pair deletion and a 3 base pair deletion wherein the resulting nucleotide sequence is SEQ ID NO:18;
a 111 base pair modification wherein the resulting nucleotide sequence is SEQ ID NO:19;
a 10 base pair deletion and a 3 base pair deletion wherein the resulting nucleotide sequence is SEQ ID NO:20;
a 10 base pair deletion and a 9 base pair deletion wherein the resulting nucleotide sequence is SEQ ID NO:21;
a 108 base pair deletion wherein the resulting nucleotide sequence is SEQ ID NO:22;
a 12 base pair deletion and a 11 base pair deletion wherein the resulting nucleotide sequence is SEQ ID NO:23;
an 18 base pair deletion and a 7 base pair deletion wherein the resulting nucleotide sequence is SEQ ID NO:24;
a 6 base pair deletion and a 4 base pair deletion wherein the resulting nucleotide sequence is SEQ ID NO:25;
a 624 base pair deletion and a 24 base pair deletion wherein the resulting nucleotide sequence is SEQ ID NO:26;
an 8 base pair deletion wherein the resulting nucleotide sequence is SEQ ID NO:27;
a 9 base pair deletion and a 7 base pair deletion wherein the resulting nucleotide sequence is SEQ ID NO:28;
a 1 base pair deletion wherein the resulting nucleotide sequence is SEQ ID NO:29;
a 10 base pair deletion wherein the resulting nucleotide sequence is SEQ ID NO:30;
a 4 base pair deletion wherein the resulting nucleotide sequence is SEQ ID NO:31;
a 7 base pair deletion wherein the resulting nucleotide sequence is SEQ ID NO:32;
a 7 base pair modification wherein the resulting nucleotide sequence is SEQ ID NO:33;
a 108 base pair deletion wherein the resulting nucleotide sequence is SEQ ID NO:34;
a 112 base pair deletion wherein the resulting nucleotide sequence is SEQ ID NO:35;
a 3 base pair deletion and a 4 base pair deletion wherein the resulting nucleotide sequence is SEQ ID NO:36;
a 16 base pair deletion wherein the resulting nucleotide sequence is SEQ ID NO:37;
an 8 base pair modification wherein the resulting nucleotide sequence is SEQ ID NO:38;
a 5 base pair deletion wherein the resulting nucleotide sequence is SEQ ID NO:39;
a 10 base pair deletion wherein the resulting nucleotide sequence is SEQ ID NO:40;
a 5 base pair deletion and a 10 base pair deletion wherein the resulting nucleotide sequence is SEQ ID NO:41;
a 234 base pair deletion and a 10 base pair deletion wherein the resulting nucleotide sequence is SEQ ID NO:42;
a 7 base pair deletion, a 64 base pair insertion, and a 3 base pair deletion wherein the resulting nucleotide sequence is SEQ ID NO:43;
a 234 base pair deletion and a 3 base pair deletion wherein the resulting nucleotide sequence is SEQ ID NO:44;
a 7 base pair deletion, a 64 base pair insertion, and a 10 base pair deletion wherein the resulting nucleotide sequence is SEQ ID NO:45;
a 25 base pair deletion and a 3 base pair deletion wherein the resulting nucleotide sequence is SEQ ID NO:46;
an 8 base pair deletion and a 9 base pair modification wherein the resulting nucleotide sequence is SEQ ID NO:47;
a 25 base pair deletion and a 9 base pair modification wherein the resulting nucleotide sequence is SEQ ID NO:48;
an 8 base pair deletion and a 3 base pair deletion wherein the resulting nucleotide sequence is SEQ ID NO:49; and
combinations of any thereof.

Embodiment B18 is the modified plant, plant seed, plant part, or plant cell of any one of embodiments B1 to B17, wherein the plant, plant seed, plant part, or plant cell comprises a modification in at least one allele of the ZmDA1 gene, wherein the modification is comprised within a genomic region from nucleotide position 3434 to nucleotide position 6811 with reference to sequence SEQ ID NO:3.

Embodiment B19-1 is the modified plant, plant seed, plant part, or plant cell of any one of embodiments B1 to B18, wherein the plant, plant seed, plant part, or plant cell comprises a modification in at least one allele of the ZmDA1 gene, wherein the modification is comprised within a genomic region from nucleotide position 3650 to nucleotide position 4437 with reference to sequence SEQ ID NO:3.

Embodiment B19-2 is the modified plant, plant seed, plant part, or plant cell of any one of embodiments B1 to B18, wherein the plant, plant seed, plant part, or plant cell comprises a modification in at least one allele of the ZmDA1 gene, wherein the modification is comprised within a genomic region from nucleotide position 3650 to nucleotide position 4443 with reference to sequence SEQ ID NO:3.

Embodiment B20 is the modified plant, plant seed, plant part, or plant cell of any one of embodiments B18 to B19-2, wherein the modification comprises a deletion of at least 1, at least 3, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 300, at least 600 or at least 650 consecutive nucleotides.

Embodiment B21 is the modified plant, plant seed, plant part, or plant cell of any one of embodiments B14 to B20, wherein the plant, plant seed, plant part, or plant cell comprises a chromosomal sequence in the ZmDA1 gene that has at least 70%, at least 75%, at least 80%, at least 85%, at least 90%, at least 95%, at least 96%, at least 97%, at least 98%, at least 99%, at least 99.5%, or 100% sequence identity to SEQ ID NO:3 in the regions outside of the modification, the deletion, the insertion, the substitution, the inversion, or the duplication.

Embodiment B22 is the modified plant, plant seed, plant part, or plant cell of any one of embodiments B1 to B21, wherein the plant, plant seed, plant part, or plant cell comprises a polynucleotide sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO: 28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49.

Embodiment B23 is the modified plant, plant seed, plant part, or plant cell of any one of embodiments B1 to B22, wherein the modification disrupts or alters the activity of ZmDA1, as compared to the activity of ZmDA1 in an otherwise identical plant, plant seed, plant part, or plant cell that lacks the modification.

Embodiment B24 is the modified plant, plant seed, plant part, or plant cell of embodiment B23, wherein the modification alters peptidase activity of ZmDA1.

Embodiment B25 is the modified plant, plant seed, plant part, or plant cell of any one of embodiments B1 to B24, wherein the modification confers an altered phenotype to the plant, as compared to the phenotype of an otherwise identical plant that lacks the modification.

Embodiment B26 is the modified plant, plant seed, plant part, or plant cell of embodiment B25, wherein the altered phenotype comprises an increase in number of kernels per ear, number of kernels per longitudinal row of ear, ear area, ear length, yield, grain yield estimate per plant, grain yield estimate, or combinations of any thereof, as compared to the phenotype of an otherwise identical plant that lacks the modification.

Embodiment B27 is the modified plant, plant seed, plant part, or plant cell of any one of embodiments B1 to B26, wherein the modified plant exhibits increased yield, grain yield estimate per plant, grain yield estimate, or combinations of any thereof, as compared to an otherwise identical plant that lacks the modification.

Embodiment B28-1 is a polynucleotide sequence comprising a sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO: 28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49.

Embodiment B28-2 is the polynucleotide sequence of embodiment B28-1, wherein the sequence is a modified endogenous ZmDA1 gene.

Embodiment B29 is a guide RNA comprising a polynucleotide sequence selected from the group consisting of SEQ ID NO:8, SEQ ID NO:9, SEQ ID NO:10.

Embodiment B30 is a method for producing a corn plant comprising a modified ZmDA1 gene, the method comprising:
a) introducing a modification into at least one target site in an endogenous ZmDA1 gene of a corn plant cell that reduces or disrupts the activity of ZmDA1;
b) identifying and selecting one or more corn plant cells of step (a) comprising said modification in said ZmDA1 gene; and
c) regenerating at least a first plant from said one or more cells selected in step (b) or a descendent thereof comprising said modification.

Embodiment B31 is the method of embodiment B30, wherein the target site is located in a coding and/or non-coding region of said endogenous ZmDA1 gene.

Embodiment B32 is the method of embodiment B30, wherein the modification is in a region of said ZmDA1 gene comprising a coding sequence for a LIM domain.

Embodiment B33 is the method of embodiment B30, wherein the modification is in a region of said ZmDA1 gene comprising a coding sequence for a LIM-like domain.

Embodiment B34-1 is the method of embodiment B30, wherein the modification is in a region of said ZmDA1 gene comprising a coding sequence for a C-terminal DA1-like region.

Embodiment B34-2 is the method of embodiment B30, wherein the modification is in a region of said ZmDA1 gene comprising a coding sequence for a LIM-domain, and/or a LIM-like domain and/or a C-terminal DA1-like domain or a combination of any thereof.

Embodiment B35 is the method of any one of embodiments B30 to B34-2, wherein introducing the modification comprises use of at least one site-specific genome modification enzyme in said plant cell.

Embodiment B36 is the method of embodiment B35, wherein the site-specific genome modification enzyme is selected from the group consisting of: an RNA-guided nuclease, a zinc-finger nuclease, a meganuclease, a TALE-nuclease, a recombinase, a transposase, and combinations of any thereof.

Embodiment B37 is the method of any one of embodiments B35 to B36, wherein the site-specific genome modification enzyme is an RNA-guided nuclease comprising a Cas nuclease, a Cpf1 nuclease, or a variant of either thereof.

Embodiment B38 is the method of any one of embodiments B35 to B37, wherein the site-specific genome modification enzyme creates at least one strand break at the target site.

Embodiment B39 is the method of any one of embodiments B30 to B38, wherein the modification is selected from the group consisting of a substitution, an insertion, an inversion, a deletion, a duplication, and a combination thereof.

Embodiment B40 is the method of embodiment B39, wherein the modification is a deletion, substitution, insertion, or a combination thereof.

Embodiment B41 is the method of embodiment B40, wherein the deletion comprises a region of at least 1, at least 3, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 300, at least 600 or at least 650 consecutive nucleotides.

Embodiment B42 is a method for producing a hybrid corn plant comprising a modified ZmDA1 gene, the method comprising crossing a corn plant comprising a modified ZmDA1 gene with a second, non-isogenic corn plant to produce a F₁ hybrid corn plant, wherein the modified ZmDA1 gene confers an altered phenotype to the hybrid corn plant as compared to the phenotype of an otherwise isogenic hybrid corn plant that lacks the modification.

Embodiment B43 is the method of embodiment B42 wherein the second, non-isogenic corn plant lacks a modified ZmDA1 gene.

Embodiment B44 is a modified plant, plant seed, plant part, or plant cell, wherein the plant, plant seed, plant part, or plant cell comprises a polynucleotide sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO: 28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49.

Embodiment B45-1 is a modified plant, plant seed, plant part, or plant cell, wherein the plant, plant seed, plant part, or plant cell comprises a polynucleotide sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO: 28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59.

Embodiment B45-2 is the modified plant, plant seed, plant part, or plant cell of embodiment B44 or B45-1, wherein the modified plant, plant seed, plant part, or plant cell is a modified corn plant, corn plant seed, corn plant part, or corn plant cell.

Embodiment B46 is a modified corn plant, plant seed, plant part, or plant cell, comprising a genomic modification in at least one allele of an endogenous ZmDA1 gene.

Embodiment B47 is the modified corn plant, plant seed, plant part, or plant cell of embodiment B46, wherein the modified allele of the endogenous ZmDA1 gene reduces or disrupts the activity of ZmDA1, as compared to the activity of ZmDA1 in an otherwise identical corn plant, corn plant seed, corn plant part, or corn plant cell that lacks the modification.

Embodiment B48 is the modified corn plant, plant seed, plant part, or plant cell of any one of embodiments B46 or B47, wherein the modified allele confers an altered phenotype to the plant, as compared to the phenotype of an otherwise identical plant that lacks the modification.

Embodiment B49 is the modified plant, plant seed, plant part, or plant cell of any one of embodiments B46 to B48, wherein the altered phenotype comprises an increase in number of kernels per ear, number of kernels per longitudinal row of ear, ear area, ear length, yield, grain yield estimate per plant, grain yield estimate, or combinations of any thereof, as compared to the phenotype of an otherwise identical plant that lacks the modification.

Embodiment B50 is the modified plant, plant seed, plant part, or plant cell of any one of embodiments B46 to B49, wherein the modification is located at about 2375 nucleotides or more upstream from the 3′ end of reference sequence SEQ ID NO:3.

Embodiment B51 is the modified plant, plant seed, plant part, or plant cell of any one of embodiments B46 to B49, wherein the modification is located at about 2369 nucleotides or more upstream from the 3′ end of reference sequence SEQ ID NO:3.

Embodiment B52 is the modified plant, plant seed, plant part, or plant cell of any one of embodiments B46 to B51, wherein the plant, plant seed, plant part, or plant cell comprises a modification in at least one allele of the ZmDA1 gene, wherein the modification is comprised within a genomic region from nucleotide position 3434 to nucleotide position 6811 with reference to sequence SEQ ID NO:3.

Embodiment B53 is the modified plant, plant seed, plant part, or plant cell of any one of embodiments B46 to B52, wherein the plant, plant seed, plant part, or plant cell comprises a modification in at least one allele of the ZmDA1 gene, wherein the modification is comprised within a genomic region from nucleotide position 3650 to nucleotide position 4437 with reference to sequence SEQ ID NO:3.

Embodiment B54 is the modified plant, plant seed, plant part, or plant cell of any one of embodiments B46 to B52, wherein the plant, plant seed, plant part, or plant cell comprises a modification in at least one allele of the ZmDA1 gene, wherein the modification is comprised within a genomic region from nucleotide position 3650 to nucleotide position 4443 with reference to sequence SEQ ID NO:3.

Embodiment B55 is the modified corn plant, plant seed, plant part, or plant cell of any one of embodiments B46 to B54, wherein the plant, plant seed, plant part, or plant cell comprises a polynucleotide sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO: 28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49.

Embodiment B56 is the modified corn plant, plant seed, plant part, or plant cell of any one of embodiments B46 to B54, wherein the plant, plant seed, plant part, or plant cell comprises a polynucleotide sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO: 28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59.

As provided herein, genomic edits of the ZmDA1 gene can be used to develop high yield corn through modulation of the ubiquitin pathway. Production of non-functional interfering proteins in corn plants as performed with the ZmDA1 gene edits provides a unique strategy and significant advance toward increasing corn yield.

Having described the present disclosure in detail, it will be apparent that modifications, variations, and equivalent embodiments are possible without departing from the spirit and scope of the present disclosure as described herein and in the appended claims. Furthermore, it should be appreciated that all examples in the present disclosure are provided as non-limiting examples.

What is claimed is:

1. A modified corn plant, corn plant seed, corn plant part, or corn plant cell, comprising a genomic modification that reduces or disrupts the activity of ZmDA1, as compared to the activity of ZmDA1 in an otherwise identical corn plant, corn plant seed, corn plant part, or corn plant cell that lacks the modification.

2. The modified plant, plant seed, plant part, or plant cell of claim 1, wherein the modification is present in at least one allele of an endogenous ZmDA1 gene.

3. The modified plant, plant seed, plant part, or plant cell of claim 1, wherein the genomic modification is in an endogenous ZmDA1 gene encoding a protein having at least 70%, at least 75%, at least 80%, at least 85%, at least 90%, at least 95%, at least 96%, at least 97%, at least 98%, at least 99%, at least 99.5%, or 100% sequence identity to SEQ ID NO:2.

4. The modified plant, plant seed, plant part, or plant cell of claim 2, wherein the modification is:
i) in a transcribable region of the ZmDA1 gene;
ii) in a region of said ZmDA1 gene comprising a coding sequence for a LIM domain;
iii) in a region of said ZmDA1 gene comprising a coding sequence for a LIM-like domain;
iv) in a region of said ZmDA1 gene comprising a coding sequence for a C-terminal DA1-like domain; or
v) a combination of any thereof.

5. The modified plant, plant seed, plant part, or plant cell of claim 4, wherein the modification is in:
i) an exon region of said ZmDA1 gene;
ii) an intron region of said ZmDA1 gene; or
iii) an exon region and an intron region of said ZmDA1 gene.

6. The modified plant, plant seed, plant part, or plant cell of claim 1, wherein:
i) the plant, plant seed, plant part, or plant cell is heterozygous for the modification;
ii) the plant, plant seed, plant part, or plant cell is homozygous for the modification;
iii) the plant, plant seed, plant part, or plant cell comprises a polynucleotide sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO: 28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, and SEQ ID NO:49; or
iv) the modified plant exhibits increased yield, grain yield estimate per plant, grain yield estimate, or combinations of any thereof, as compared to an otherwise identical plant that lacks the modification.

7. The modified plant, plant seed, plant part, or plant cell of claim 6, wherein the plant, plant seed, plant part, or plant cell comprises a first modification in a first allele of the ZmDA1 gene and a second modification in a second allele of the ZmDA1 gene, the first modification and the second modification being different from one another.

8. The modified plant, plant seed, plant part, or plant cell of claim 1, wherein the modification:
i) comprises a deletion, an insertion, a substitution, an inversion, a duplication, or a combination of any thereof;
ii) is located at about 2369 nucleotides or more upstream from the 3′ end of reference sequence SEQ ID NO:3;
iii) is located at about 3650 nucleotides or more downstream from the 5′ end of reference sequence SEQ ID NO:3;
iv) comprises a deletion, a substitution, an insertion, or a combination of any thereof;
v) disrupts or alters the activity of ZmDA1, as compared to the activity of ZmDA1 in an otherwise identical plant, plant seed, plant part, or plant cell that lacks the modification; or
vi) confers an altered phenotype to the plant, as compared to the phenotype of an otherwise identical plant that lacks the modification.

9. The modified plant, plant seed, plant part, or plant cell of claim 2 wherein the plant, plant seed, plant part, or plant cell comprises a modification in at least one allele of the ZmDA1 gene, wherein the modification is selected from the group consisting of:
a 115 base pair deletion wherein the resulting nucleotide sequence is SEQ ID NO:11;
a 10 base pair deletion and a 4 base pair deletion wherein the resulting nucleotide sequence is SEQ ID NO:12;
a 7 base pair deletion and a 6 base pair deletion wherein the resulting nucleotide sequence is SEQ ID NO:13;
an 8 base pair modification and an 11 base pair deletion wherein the resulting nucleotide sequence is SEQ ID NO:14;
a 10 base pair deletion and a 6 base pair deletion wherein the resulting nucleotide sequence is SEQ ID NO:15;
a 113 base pair deletion wherein the resulting nucleotide sequence is SEQ ID NO:16;
a first 6 base pair deletion and a second 6 base pair deletion wherein the resulting nucleotide sequence is SEQ ID NO:17;
a 29 base pair deletion and a 3 base pair deletion wherein the resulting nucleotide sequence is SEQ ID NO:18;
a 111 base pair modification wherein the resulting nucleotide sequence is SEQ ID NO:19;
a 10 base pair deletion and a 3 base pair deletion wherein the resulting nucleotide sequence is SEQ ID NO:20;
a 10 base pair deletion and a 9 base pair deletion wherein the resulting nucleotide sequence is SEQ ID NO:21;
a 108 base pair deletion wherein the resulting nucleotide sequence is SEQ ID NO:22;
a 12 base pair deletion and a 11 base pair deletion wherein the resulting nucleotide sequence is SEQ ID NO:23;
an 18 base pair deletion and a 7 base pair deletion wherein the resulting nucleotide sequence is SEQ ID NO:24;
a 6 base pair deletion and a 4 base pair deletion wherein the resulting nucleotide sequence is SEQ ID NO:25;
a 624 base pair deletion and a 24 base pair deletion wherein the resulting nucleotide sequence is SEQ ID NO:26;
an 8 base pair deletion wherein the resulting nucleotide sequence is SEQ ID NO:27;
a 9 base pair deletion and a 7 base pair deletion wherein the resulting nucleotide sequence is SEQ ID NO:28;
a 1 base pair deletion wherein the resulting nucleotide sequence is SEQ ID NO:29;
a 10 base pair deletion wherein the resulting nucleotide sequence is SEQ ID NO:30;
a 4 base pair deletion wherein the resulting nucleotide sequence is SEQ ID NO:31;
a 7 base pair deletion wherein the resulting nucleotide sequence is SEQ ID NO:32;
a 7 base pair modification wherein the resulting nucleotide sequence is SEQ ID NO:33;
a 108 base pair deletion wherein the resulting nucleotide sequence is SEQ ID NO:34;
a112 base pair deletion wherein the resulting nucleotide sequence is SEQ ID NO:35;
a 3 base pair deletion and a 4 base pair deletion wherein the resulting nucleotide sequence is SEQ ID NO:36;
a 16 base pair deletion wherein the resulting nucleotide sequence is SEQ ID NO:37;
an 8 base pair modification wherein the resulting nucleotide sequence is SEQ ID NO:38;
a 5 base pair deletion wherein the resulting nucleotide sequence is SEQ ID NO:39;
a 10 base pair deletion wherein the resulting nucleotide sequence is SEQ ID NO:40;
a5 base pair deletion and a 10 base pair deletion wherein the resulting nucleotide sequence is SEQ ID NO:41;
a 234 base pair deletion and a 10 base pair deletion wherein the resulting nucleotide sequence is SEQ ID NO:42;
a 7 base pair deletion, a 64 base pair insertion, and a 3 base pair deletion wherein the resulting nucleotide sequence is SEQ ID NO:43;
a 234 base pair deletion and a 3 base pair deletion wherein the resulting nucleotide sequence is SEQ ID NO:44;
a 7 base pair deletion, a 64 base pair insertion, and a 10 base pair deletion wherein the resulting nucleotide sequence is SEQ ID NO:45;
a 25 base pair deletion and a 3 base pair deletion wherein the resulting nucleotide sequence is SEQ ID NO:46;
an 8 base pair deletion and a 9 base pair modification wherein the resulting nucleotide sequence is SEQ ID NO:47;
a 25 base pair deletion and a 9 base pair modification wherein the resulting nucleotide sequence is SEQ ID NO:48;
an 8 base pair deletion and a 3 base pair deletion wherein the resulting nucleotide sequence is SEQ ID NO:49; and
combinations of any thereof.

10. The modified plant, plant seed, plant part, or plant cell of claim 2, wherein the plant, plant seed, plant part, or plant cell comprises a modification in at least one allele of the ZmDA1 gene, wherein:
i) the modification is comprised within a genomic region from about nucleotide position 3434 to about nucleotide position 6811 with reference to sequence SEQ ID NO:3; or
ii) the modification is comprised within a genomic region from about nucleotide position 3650 to about nucleotide position 4437 of reference sequence SEQ ID NO:3; or
iii) the modification is comprised within a genomic region from about nucleotide position 3650 to about nucleotide position 4443 of reference sequence SEQ ID NO:3.

11. The modified plant, plant seed, plant part, or plant cell of claim 8, wherein:
i) the modification comprises a deletion of at least 1, at least 3, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 300, at least 600 or at least 650 consecutive nucleotides;
ii) the plant, plant seed, plant part, or plant cell comprises a chromosomal sequence in the ZmDA1 gene that has at least 70%, at least 75%, at least 80%, at least 85%, at least 90%, at least 95%, at least 96%, at least 97%, at least 98%, at least 99%, at least 99.5%, or 100% sequence identity to SEQ ID NO:3 in the regions outside of the modification;
iii) the modification alters peptidase activity of ZmDA1; or
iv) the altered phenotype comprises an increase in number of kernels per ear, number of kernels per longitudinal row of ear, ear area, ear length, yield, grain yield estimate per plant, grain yield estimate, or combinations of any thereof, as compared to the phenotype of an otherwise identical plant that lacks the modification.

12. The modified plant, plant seed, plant part, or plant cell of claim 8, wherein:
i) the modification is comprised within a genomic region from about nucleotide position 4325 to about nucleotide position 4433 with reference to sequence SEQ ID NO:3;
ii) the modification is comprised within a genomic region from about nucleotide position 4107 to about nucleotide position 4437 of reference sequence SEQ ID NO:3; or
iii) the modification is comprised within a genomic region from about nucleotide position 4326 to about nucleotide position 4433 of reference sequence SEQ ID NO:3.

13. A polynucleotide sequence comprising a sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO: 28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, and SEQ ID NO:49.

14. The polynucleotide sequence of claim 13, wherein the sequence is a modified endogenous ZmDA1 gene.

15. A method for producing a corn plant comprising a modified ZmDA1 gene, the method comprising:
a) introducing a modification into at least one target site in an endogenous ZmDA1 gene of a corn plant cell that reduces or disrupts the activity of ZmDA1;
b) identifying and selecting one or more corn plant cells of step (a) comprising said modification in said ZmDA1 gene; and
c) regenerating at least a first plant from said one or more cells selected in step (b) or a descendent thereof comprising said modification.

16. The method of claim 15, wherein:
i) the target site is located in a coding and/or non-coding region of said endogenous ZmDA1 gene;
ii) the modification is in a region of said ZmDA1 gene comprising a coding sequence for a LIM domain;
iii) the modification is in a region of said ZmDA1 gene comprising a coding sequence for a LIM-like domain;
iv) the modification is in a region of said ZmDA1 gene comprising a coding sequence for a C-terminal DA1-like region;
v) introducing the modification comprises use of at least one site-specific genome modification enzyme in said plant cell;
vi) the modification is selected from the group consisting of a substitution, an insertion, an inversion, a deletion, a duplication, and a combination thereof; or
vii)the modification is a deletion, substitution, insertion, or a combination thereof.

17. The method of claim 16, wherein the site-specific genome modification enzyme:
i) is selected from the group consisting of: an RNA-guided nuclease, a zinc-finger nuclease, a meganuclease, a TALE-nuclease, a recombinase, a transposase, and combinations of any thereof;
ii) is an RNA-guided nuclease comprising a Cas nuclease, a Cpf1 nuclease, or a variant of either thereof; or
iii) creates at least one strand break at the target site.

18. The method of claim 16, wherein the deletion comprises a region of at least 1, at least 3, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, least 150, at least 200, at least 300, at least 600 or at least 650 consecutive nucleotides.

19. A method for producing a hybrid corn plant comprising a modified ZmDA1 gene, the method comprising crossing a corn plant comprising a modified ZmDA1 gene with a second, non-isogenic corn plant to produce a F₁ hybrid corn plant, wherein the modified ZmDA1 gene confers an altered phenotype to the hybrid corn plant as compared to the phenotype of an otherwise isogenic hybrid corn plant that lacks the modification.

20. The method of claim 19 wherein the second, non-isogenic corn plant lacks a modified ZmDA1 gene.
